(19) United States
(12) Patent Application Publication
(10) Pub. No.: US 2024/0277350 A1
(43) Pub. Date: Aug. 22, 2024

(54) APPARATUS, SYSTEM, AND METHOD FOR PATIENT-SPECIFIC HARVESTING GUIDE

(71) Applicant: Treace Medical Concepts, Inc., Ponte Vedra, FL (US)

(72) Inventors: James Q. Spitler, Winter Garden, FL (US); Lee M. Hlad, Waycross, GA (US); Adam D. Perler, St. Petersburg, FL (US); Andrew C. Khan, Clermont, FL (US)

(21) Appl. No.: 18/583,504

(22) Filed: Feb. 21, 2024

Related U.S. Application Data
(60) Provisional application No. 63/447,257, filed on Feb. 21, 2023.

(51) Int. Cl. A61B 17/16; A61B 34/10

(52) U.S. Cl. CPC A61B 17/1635; A61B 17/1682; A61B 34/10; A61B 2034/108

(57) ABSTRACT

An apparatus, system, and method are disclosed for remediating a condition present in a patient. In some implementations, the apparatus may include a body. Also, the apparatus may include a bone attachment feature. Furthermore, the apparatus may include a first resection feature configured to guide a first osteotomy of a donor bone. In addition, the apparatus may include a second resection feature configured to guide a second osteotomy of the donor bone, the second osteotomy offset from the first resection feature by a dimension that satisfies a predefined bone graft dimension.

### CROSS-REFERENCE TO RELATED APPLICATIONS

This application claims the benefit of and priority to U.S. Provisional Application No. 63/447,257, filed Feb. 21, 2023, which is hereby incorporated by reference in its entirety.

### TECHNICAL FIELD

The present disclosure relates to surgical devices, systems, instruments, and methods. More specifically, the present disclosure relates to patient-specific guides, implants, instruments, and/or methods of designing and using the same.

### BACKGROUND

Various bone conditions may be corrected using surgical procedures, in which one or more tendons, ligaments, and/or bones may be cut, replaced, repositioned, reoriented, reattached, fixated and/or fused. These surgical procedures require the surgeon to properly locate, position, and/or orient one or more osteotomy cuts, fixation guides, fixators, bone tunnels, points of attachment for ends of grafts or soft tissue and the like.

Hallux valgus or bunion conditions can be a source of discomfort, pain, and inconvenience for patients. Among a variety of different approaches for dealing with hallux valgus or bunion conditions, a Lapidus arthrodesis or simply Lapidus procedure is a common surgical procedure to address this condition.

A patient may present with a condition that can be remediated using a surgical procedure that calls for a bone graft to be inserted between one or more bones, bone fragments, cut faces, or within a joint. For example, where a patient and surgeon determine that one bone should be lengthened. Or, in another example, where a surgical procedure such as a Lapidus surgical procedure, arthroplasty, or arthrodesis surgical procedure are expected to result in one appendage being unacceptably shorter than another. In these situations, a patient and/or surgeon may plan to harvest a bone segment or bone block to insert in an opening between two or more bone fragments to remediate a difference in length. In particular, the surgeon may plan to harvest the bone segment, bone fragment, or bone block, from a site of the patient.

Determining and locating an optimal or desired location and trajectory for one or more steps of a bone graft harvesting surgical procedure can be challenging, given conventional techniques and instruments. Advancements in medical imaging, preoperative planning, modeling, and the like have led to improvements that help surgeons execute a Lapidus surgical procedure. However, apparatus, systems, and/or methods for harvesting bone grafts from a patient are lacking or have limitations. What is needed is a solution that facilitates harvesting a bone graft from a patient for use in connection with another surgical procedure. The present disclosure provides such a solution.

### SUMMARY

The various apparatus, devices, systems, and/or methods of the present disclosure have been developed in response to the present state of the art, and in particular, in response to the problems and needs in the art that have not yet been fully solved by currently available technology.

In one general aspect, an apparatus may include a body. An Apparatus may also include a bone attachment feature. An apparatus may furthermore include a first resection feature configured to guide a first osteotomy of a donor bone. An apparatus may in addition include a second resection feature configured to guide a second osteotomy of the donor bone, the second osteotomy offset from the first resection feature by a dimension that satisfies a predefined bone graft dimension. Other embodiments of this aspect include corresponding computer systems, apparatus, and computer programs recorded on one or more computer storage devices, each configured to perform the actions of the methods.

Implementations may include one or more of the following features. An apparatus where the predefined bone graft dimension is one of a set of predefined dimensions determined for an opening in a planned surgical procedure. An apparatus where the body may include a bone engagement surface configured to engage a surface of the donor bone. An apparatus where the bone engagement surface may include a contour at least partially determined based on a bone model of the donor bone, the bone model defined based on medical imaging of the donor bone. An apparatus where the bone engagement surface is configured to engage at least two cortical surfaces of the donor bone.

An apparatus may include a landmark registration feature configured to engage a landmark of the donor bone. An apparatus may include a guard pin guide configured to receive a guard pin into the donor bone such that the guard pin prevent cutting donor bone beyond a boundary. An apparatus where the guard pin guide is positioned at an end of at least one of the first resection feature and the second resection feature. An apparatus may include an alternative resection feature that is offset from the first resection feature by a second dimension, the second dimension different from the dimension. An apparatus where the first resection feature and the second resection feature each extend through the body from a bone-facing side of the body to a non-bone-facing side of the body and where the first resection feature and the second resection feature extend through the body parallel to each other. An apparatus where the first resection feature and the second resection feature extend through the body at an angle such that the first osteotomy and the second osteotomy form a wedge-shaped bone graft. An apparatus where the donor bone is a calcaneus of a patient of a Lapidus surgical procedure. Implementations of the described techniques may include hardware, a method or process, or a computer tangible medium.

In one general aspect, system may include a harvesting guide having: a body; a bone attachment feature; a first resection feature, the first resection feature configured to guide a first osteotomy of a donor bone; and a second resection feature, the second resection feature configured to guide a second osteotomy of the donor bone, the first resection feature extending through the body parallel to the second resection feature, the first resection feature separated from the second resection feature by a distance defined based on a patient-specific bone graft insertion site. A system may also include a guard configured to prevent cutting the donor bone beyond a boundary. Other embodiments of this aspect include corresponding computer systems, apparatus, and computer programs recorded on one or more computer storage devices, each configured to perform the actions of the methods.

Implementations may include one or more of the following features. A system where the harvesting guide may include: a third resection feature that extends from a bone-facing side to a non-bone-facing side of the body; where the third resection feature guides a third osteotomy that connects the first osteotomy and the second osteotomy. A system where the third resection feature may include an edge. A system may include a jig configured to guide shaping of a bone graft resected from the donor bone using the harvesting guide. Implementations of the described techniques may include hardware, a method or process, or a computer tangible medium.

In one general aspect, method may include performing an osteotomy on a patient using an instrument that forms an opening having a set of predefined dimensions within one or more bones of a patient. A method may also include deploying a harvesting guide on to a donor bone, the harvesting guide having: a body; a bone attachment feature; a first resection feature, the first resection feature configured to guide an osteotomy of the donor bone; a second resection feature, the4avigad resection feature configured to guide an osteotomy of the donor bone; and where the first resection feature and second resection feature are configured relative to each other such that an osteotomy formed using the first resection feature and an osteotomy formed using the second resection feature facilitates resection of a bone graft having at least one dimension that substantially matches a dimension of the set of predefined dimensions.

A method may furthermore include resecting the bone graft from the donor bone using the first resection feature and the second resection feature of the harvesting guide. Other embodiments of this aspect include corresponding computer systems, apparatus, and computer programs recorded on one or more computer storage devices, each configured to perform the actions of the methods.

Implementations may include one or more of the following features. A method may include preparing the bone graft for reduction within the opening; and reducing at least one bone fragment with the bone graft within the opening. A method where the body may include a bone engagement surface configured to engage at least two cortical surfaces of the donor bone. Implementations of the described techniques may include hardware, a method or process, or a computer tangible medium.

In one general aspect, method may include providing a set of predefined dimensions for an opening that is to receive a bone graft, the set of predefined dimensions unique to a patient. A method may also include determining a position on a donor bone for a bone graft harvest site. A method may furthermore include developing a patient-specific harvesting guide model having a bone engagement surface configured to engage with a surface of the bone graft harvest site. A method may in addition include fabricating a patient-specific harvesting guide based on the patient-specific harvesting guide model. A method may moreover include providing the patient-specific harvesting guide for a surgical procedure. Other embodiments of this aspect include corresponding computer systems, apparatus, and computer programs recorded on one or more computer storage devices, each configured to perform the actions of the methods.

### BRIEF DESCRIPTION OF THE DRAWINGS

The advantages, nature, and additional features of exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure's scope, the exemplary embodiments of the disclosure will be described with additional specificity and detail through use of the accompanying drawings.

FIG. 1A is a flowchart diagram depicting a method for remediating a condition, according to one embodiment.

FIG. 1B is a flowchart diagram depicting a method for remediating a condition, according to one embodiment.

FIG. 2A is a dorsal perspective view of bones of a foot.

FIG. 2B is a lateral perspective view of bones of a foot.

FIG. 2C is a medial perspective view of bones of a foot.

FIG. 2D is a dorsal perspective view of bones of a foot.

FIG. 2E is a view of a foot illustrating common planes of reference for a human foot.

FIG. 3 is a flowchart diagram depicting a method for generating one or more patient-specific instruments configured to address a bone condition, according to one embodiment.

FIG. 4 illustrates an exemplary system configured to generate one or more patient-specific instruments configured to address a bone condition, according to one embodiment.

FIG. 5 illustrates an exemplary apparatus configured to determine a location and/or a trajectory for instrumentation, according to one embodiment.

FIG. 6 illustrates an exemplary provision module configured to provide a preliminary guide model, according to one embodiment.

FIG. 7 illustrates an exemplary design module configured to design a patient-specific guide model, according to one embodiment.

FIG. 8 illustrates an exemplary system configured to generate one or more patient-specific instruments configured to address a bone condition, according to one embodiment.

FIG. 9 illustrates a method for providing a patient-specific harvesting guide, according to one embodiment.

FIG. 10 illustrates a method for harvesting a bone graft for a surgical procedure, according to one embodiment.

FIG. 11 illustrates an exemplary system, according to one embodiment.

FIG. 12 illustrates an exemplary system, according to one embodiment.

FIGS. 13A-13G are top perspective, top, bottom, posterior, anterior, superior, and inferior views respectively, of a harvesting guide according to one embodiment.

FIGS. 13H-13N are views of alternative embodiments of a harvesting guide according to certain embodiments.

FIGS. 14A-14G illustrate different views of stages of a surgical procedure, according to one embodiment.

FIG. 14H illustrates an example cut face for use with one embodiment.

FIG. 14I illustrates an example jig for use with one embodiment.

FIGS. 15A-15O illustrate different views and information for a planned surgical procedure that includes one or more osteotomies as well as harvesting of an autograft using a patient-specific harvesting guide, according to one embodiment.

### DETAILED DESCRIPTION

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and method is not intended to limit the scope of the disclosure but is merely representative of exemplary embodiments.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature can pass into the other feature.

As used herein, “coupling”, “coupling member”, or “coupler” refers to a mechanical device, apparatus, member, component, system, assembly, or structure, that is organized, configured, designed, arranged, or engineered to connect, or facilitate the connection of, two or more parts, objects, or structures. In certain embodiments, a coupling can connect adjacent parts or objects at their ends. In certain embodiments, a coupling can be used to connect two shafts together at their ends for the purpose of transmitting power. In other embodiments, a coupling can be used to join two pieces of rotating equipment while permitting some degree of misalignment or end movement or both. In certain embodiments, couplings may not allow disconnection of the two parts, such as shafts during operation. (Search “coupling” on Wikipedia.com Jul. 26, 2021. CC-BY-SA 3.0 Modified. Accessed Jul. 27, 2021.) A coupler may be flexible, semiflexible, pliable, elastic, or rigid. A coupler may join two structures either directly by connecting directly to one structure and/or directly to the other or indirectly by connecting indirectly (by way of one or more intermediary structures) to one structure, to the other structure, or to both structures.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Standard medical planes of reference and descriptive terminology are employed in this disclosure. While these terms are commonly used to refer to the human body, certain terms are applicable to physical objects in general. A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body from the side which has a particular condition or structure. Proximal means toward the trunk of the body. Proximal may also mean toward a user, viewer, or operator. Distal means away from the trunk. Distal may also mean away from a user, viewer, or operator. Dorsal means toward the top of the foot or other body structure. Plantar means toward the sole of the foot or toward the bottom of the body structure.

Antegrade means forward moving from a proximal location/position to a distal location/position or moving in a forward direction. Retrograde means backward moving from a distal location/position to a proximal location/position or moving in a backwards direction. Sagittal refers to a midline of a patient's anatomy, which divides the body into left or right halves. The sagittal plane may be in the center of the body, splitting it into two halves. Prone means a body of a person lying face down. Supine means a body of a person lying face up.

“Patient-specific guide” refers to a guide designed, engineered, and/or fabricated for use with a specific patient. In one aspect, a patient-specific guide is unique to a patient and may include features unique to the patient such as a surface contour or other features.

“Patient-specific cutting guide” refers to a cutting guide designed, engineered, and/or fabricated for use with a specific patient. In one aspect, a patient-specific cutting guide is unique to a patient and may include features unique to the patient such as a surface contour or other features.

“Patient-specific resection guide” refers to a guide designed, engineered, and/or fabricated for use in resection for a specific patient. In one aspect, a patient-specific resection guide is unique to a patient and may include features unique to the patient such as a surface contour or other features.

“Harvesting guide”, “Graft harvesting guide”, or “Harvester” refers to an instrument designed, engineered, and/or fabricated for facilitating removal of an autograft (autologous), allograft, and/or xenograft from a harvesting site. In one aspect, a harvesting guide is unique to a single patient and may include features unique to the patient such as a surface contour, bone engagement surface, resection guides for resecting a graft of a size, shape, configuration, and/or orientation unique to the needs of a single patient, or other features. In one aspect, a harvesting guide includes one or more patient-specific features and/or aspects designed, engineered, and/or fabricated for harvesting a graft for a specific surgical procedure for a specific patient. Alternatively, the harvesting guide may not be patient-specific.

“Patient specific instrument” (PSI) refers to a structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient. In certain aspects, one patient. In one aspect, a patient specific instrument is unique to a single patient and may include features unique to the patient such as a surface contour, component position, component orientation, and/or other features. In other aspects, one patient specific instrument may be useable with a number of patients having a particular class of characteristics.

As used herein, “allograft” refers to a type of tissue and/or organ graft in which the tissue or organ of the graft is from a donor of the same species but not the same genotype. The tissue may be soft tissue such as skin, ligament, tendon, fascia, fat muscle, fibrous tissue, blood vessels, lymph vessels, or nerves or hard tissue such as bone, tooth enamel, dentin, cementum, or cartilage. Bone grafts may be of an allograft type or of a mixture of other graft types including allograft, autograft (autologous), and xenograft. Autograft refers to a type of tissue and/or organ graft in which the tissue or organ of the graft is from the patient. Xenograft refers to a type of tissue and/or organ graft in which the tissue or organ of the graft is from a donor of another species.

“Instrument” refers to any apparatus, device, of object that can be used by a user. An instrument may be used for a specific or a generic purpose. An instrument may also be referred to as instrumentation. Instrumentation may refer to a single instrument and/or a plurality of instruments. An instrument may be specifically designed, constructed or fabricated for use by a specific user and/or for a single use. A patient specific instrument is one example of an instrument.

“Patient-specific bone graft insertion site” refers to a location or area within a patient that is being prepared to or is prepared to receive a graft. Often the patient specific bone graft insertion site is an opening in tissue, an organ, a joint, one or more osteotomies, a set of bones, or a single bone.

“Bone graft harvest site” refers to a location or area within a donor, within or on donor anatomy, or within a graft source that will be used to obtain a bone graft.

“Donor bone” refers to a bone that is used as a source for a bone graft. The bone may be a bone of a patient, in which case the bone graft is an autograft. The donor bone may be a bone of a donor who is of the same species as the patient but not the same genotype, in which case the bone graft is an allograft. The donor bone may be a bone of a donor who is of another species as the patient, in which case the bone graft is a xenograft.

“Jig” refers to a device, system, structure, and/or apparatus in manufacturing, fabrication, surgery, or other endeavors for controlling the location, path of movement, or both of either a workpiece or the tool that is operating upon the workpiece. Subsets of this general class include machining jigs, graft harvesting jigs, woodworking jigs, welder's jigs, jeweler's jigs, and many others. (Search “jig” on wordhippo.com. WordHippo, 2023. Web. Modified. Accessed Feb. 17, 2023.) In the context of a surgical procedure, a jig can facilitate resection and/or shaping of a workpiece such as a tissue graft, aka a graft.

“Positioner” or “positioning guide” refers to any structure, apparatus, surface, device, system, feature, or aspect configured to position, move, translate, manipulate, or arrange one object in relation to another. In certain embodiments, a positioner can be used for one step in surgical procedure to position, arrange, orient, and/or reduce one bone or bone fragment relative to another. In such embodiments, the positioner may be referred to as a bone positioner. In certain embodiments, the term positioner or positioning guide may be preceded by an adjective that identifies the structure, implement, component, or instrument that may be used with, positioned by, and/or guided by with the positioner. For example, a “pin positioner” may be configured to accept a pin or wire such as a K-wire and serve to position or place the pin relative to another structure such as a bone.

“Compressor” refers to any apparatus, device, or system that can function as an active compression instrument. A compressor functions to bring two objects closer to or in contact with each other.

“Post” refers to any apparatus, structure, device, system, and/or component that extends from another structure. In certain embodiments, a post can be cylindrical.

“Cut surface” or “cut face” refers to a surface of an object that is created or formed by the removal of one or more parts of the object that includes the original surface. Cut surfaces or cut faces can be created using a variety of methods, tools, or apparatuses and may be formed using a variety of removal actions, including, but not limited to, fenestrating, drilling, abrading, cutting, sawing, chiseling, digging, scrapping, and the like. Tools and/or methods used for forming a cut surface or cut face can include manual, mechanical, motorized, hydraulic, automated, robotic, and the like. In certain embodiments, the cut surface(s) are planar.

“Reference” refers to any apparatus, structure, device, system, component, marking, and/or indicator organized, configured, designed, engineered, and/or arranged to serve as a source of information or a point of comparison used to support or establish knowledge, truth, or quality. (© ChatGPT January 9 Version, Modified, accessed chat.openai.com/chat Jan. 28, 2023). In certain embodiments, a reference can serve as a starting point or initial position for one or more steps in a surgical procedure. In certain embodiments, “reference” can be combined with an adjective describing the reference. For example, a “model reference” is a reference within a model such as a computer model. A model reference refers to any feature, aspect, and/or component within a model. Examples of a model reference include, but are not limited to, a point, a plane, a line, a plurality of points, a surface, an anatomical structure, a shape, or the like. An “anatomical reference” is a reference within, on, near, or otherwise associated with an anatomical structure such as a bone. A reference (e.g., model, actual, virtual, and/or real) may also be referred to as a reference feature.

“Reference feature” refers to a feature configured for use as a point, plane, axis, or line of reference (aka a reference). A reference can be used to position, measure, orient, fixation, couple, engage, and/or align one object or structure with another object or structure. In certain embodiments, a reference or reference feature can serve as a baseline, a ground truth, a waypoint, a control point, a landmark, and/or the like. A reference feature can facilitate moving from one coordinate system to a position or orientation on or in an actual object, structure, device, apparatus, anatomical structure, or the like. Advantageously, a reference feature can coordinate objects, models, or structures in a digital or virtual model or representation with corresponding objects or structures (e.g., anatomical structures) of actual physical objects or structures. Said another way, a reference feature can serve to map from a virtual or modeled object to an actual or physical object. As used herein, “feature” refers to a distinctive attribute or aspect of something. (Search “feature” on google.com. Oxford Languages, 2021. Web. 20 Apr. 2021.) A feature may include one or more apparatuses, structures, objects, systems, sub-systems, devices, or the like. A feature may include a modifier that identifies a particular function or operation and/or a particular structure relating to the feature. Examples of such modifiers applied to a feature, include, but are not limited to, “attachment feature,” “securing feature,” “placement feature,” “protruding feature,” “engagement feature,” “disengagement feature,” “resection feature”, “guide feature”, and the like.

“Reference feature guide” refers to a guide that serves to aid in forming and/or deploying one or more reference features. Examples of reference feature guides include but are not limited to a hole, a round hole, a channel, a slot, a plurality of holes, a fence, a backstop, a guard, a fastener, a pilot hole, a blind hole, a chute, a ramp, or the like.

“Anatomical structure” refers to any part or portion of a part of a body of a person, animal, or other patient. Examples of anatomical structures, include but are not limited to, a bone, bones, soft tissue, a joint, joints, skin, hard tissue, teeth, mouth, eyes, hair, nails, fingers, toes, legs, arms, torso, vertebrae, ligaments, tendons, organs, or the like.

“Anatomical reference” or “anatomical landmark” refers to any reference or landmark that is, or is on, or is in, or is otherwise associated, with an anatomical structure. Examples of anatomical structures, include but are not limited to, a bone, bones, soft tissue, a joint, joints, skin, hard tissue, teeth, mouth, eyes, hair, nails, fingers, toes, legs, arms, torso, vertebrae, ligaments, tendons, organs, a hole, a post, a plurality of holes, a plurality of posts, or the like.

“Deformity” refers to an abnormality or deviation from the normal shape, structure, orientation, trajectory, or function of a body part. This can be due to congenital conditions, injuries, diseases, or other factors that alter the normal development or functioning of a part of the body. (© ChatGPT January 30 Version, accessed chat.openai.com/chat Feb. 7, 2023).

“Configuration” refers to an arrangement, setup, or values of one or more parts, features, settings, components, aspects, structures, or the like as a module, component, apparatus, device, system, framework, platform, dashboard, assembly, or the like. Examples of configurations can include how dials are setup on a dashboard, levers are set on a control board, switches are set within a controller, bones are arranged within a hand, foot, or limb, or the like.

“Interconnect” refers to a structure configured to join at least two other structures. In one embodiment, the interconnect may be a mechanical structure that may physically connect one structure to another structure. In other embodiments, an interconnect may be embodied as a fastener that enables permanent or temporary joining of one structure to another structure. In still other embodiments, an interconnect may be embodied as a joint or hinge configured to enable one or both structured joined by the interconnect to move relative to each other while remaining joined. In one embodiment, the interconnect may be configured to convey fluid and/or an electric signal between the at least two other structures. For example, the interconnect may comprise a channel or tube configured to convey air between a first opening and a second opening in the channel or tube. Examples of an interconnect include, but are not limited to, a pipe, a tunnel, a chamber, a channel, or the like. Other examples of an interconnect include, but are not limited to, solid material that can be additively manufactured between two structures, a snap, a hook and loop system, a spring, a tether, or the like.

As used herein, a “handle” refers to a structure used to hold, control, or manipulate a device, apparatus, component, tool, or the like. A “handle” may be designed to be grasped and/or held using one or two hands of a user.

As used herein, an “indicator” refers to an apparatus, device, component, system, assembly, mechanism, hardware, software, firmware, circuit, module, set of data, text, number, code, symbol, a mark, or logic structured, organized, configured, programmed, designed, arranged, or engineered to convey information or indicate a state, condition, mode, context, location, or position to another apparatus, device, component, system, assembly, mechanism, hardware, software, firmware, circuit, module, and/or a user of an apparatus, device, component, system, assembly, mechanism, hardware, software, firmware, circuit, module that includes, or is associated with the indicator. The indicator can include one or more of an audible signal, a token, a presence of a signal, an absence of a signal, a tactile signal, a visual signal or indication, a visual marker, a visual icon, a visual symbol, a visual code, a visual mark, and/or the like. In certain embodiments, “indicator” can be used with an adjective describing the indicator. For example, a “mode indicator” is an indicator that identifies or indicates a mode.

As used herein, a “marking” or “marker” refers to a symbol, letter, lettering, word, phrase, icon, design, color, diagram, indicator, figure, structure, device, apparatus, surface, component, system, or combination of these designed, intended, structured, organized, configured, programmed, arranged, or engineered to communication information and/or a message to a user receiving, viewing, or encountering the marking. The marking or “marker” can include one or more of a tactile signal, a visual signal or indication, an audible signal, and the like. In one embodiment, a marking may comprise a number or set letters, symbols, or words positioned on a surface, structure, color, color scheme, or device to convey a desired message or set of information.

As used herein, a “graft,” “tissue graft,” and/or “bone graft” refers to a surgical procedure to move tissue (hard and/or soft tissue) from one site to another on the body, or from another creature, without bringing its own blood supply with the tissue. Instead, a new blood supply grows in after the tissue is placed. A similar technique where tissue is transferred with the blood supply intact is called a flap. (Search ‘Graft (surgery)’ on Wikipedia.com Apr. 21, 2021. Modified. Accessed Aug. 30, 2021.) “Graft” may also be used to refer to the tissue and/or synthetic composition used for a graft surgical procedure. Bone grafting is a surgical procedure that replaces missing bone to repair bone fractures. Bone generally can regenerate completely but may require a small fracture space and/or a scaffold to do'so. Bone grafts' may be autologous (bone harvested From the patient's own body, often from the iliac crest), allograft (cadaveric bone usually obtained from a bone bank), or synthetic (often made of hydroxyapatite (HA) or other naturally occurring and biocompatible substances) with similar mechanical properties to bone. Generally, bone grafts are expected to be reabsorbed and replaced as natural bone heals over a few months' time. (Search ‘Bone Grafting’ on Wikipedia.com Apr. 21, 2021. Modified. Accessed Aug. 30, 2021.) Certain grafts may include a combination of autograft, isograft, allograft, xenograft, and/or synthetic materials in a single bone graft composition. An example of such a compositions, include but is not limited to, Demineralized bone matrix (DBM). Bone graft compositions may include bone morphogenetic proteins (BMPs).

As used herein, a “condition” refers to a state of something with regard to its appearance, quality, or working order. In certain aspects, a condition may refer to a patient's state of health or physical fitness or the state of health or physical fitness of an organ or anatomical part of a patient. In certain embodiments, a condition may refer to an illness, defect, disease, or deformity of a patient or of an organ or anatomical part of a patient. (Search “condition” on wordhippo.com. WordHippo, 2021. Web. Accessed 8 Dec. 2021. Modified.)

“Bone condition” refers to any of a variety of conditions of bones of a patient. Generally, a bone condition refers to an orientation, position, and/or alignment of one or more bones of the patient relative to other anatomical structures of the body of the patient. Bone conditions may be caused by or result from deformities, misalignment, malrotation, fractures, joint failure, and/or the like. A bone condition includes, but is not limited to, any angular deformities of one or more bone segments in either the lower or upper extremities (for example, tibial deformities, calcaneal deformities, femoral deformities, and radial deformities). Alternatively, or in addition, “bone condition” can refer to the structural makeup and configuration of one or more bones of a patient. Thus bone condition may refer to a state or condition of regions, a thickness of a cortex, bone density, a thickness and/or porosity of internal regions (e.g. whether it is calcaneus or solid) of the bone or parts of the bone such as a head, a base, a shaft, a protuberance, a process, a lamina, a foramen, and the like of a bone, along the metaphyseal region, epiphysis region, and/or a diaphyseal region.

“Data” refers to a set of information organized in a way that facilitates communication of the information to a receiver. The receiver may be a person or animal or an electronic component, circuit, assembly, or the like. Data can be represented as signal or values represented in any numbering and/or alphabet system. Data can be stored in one representation in an analog or digital format and conveyed to a receiver in another format suitable for the receiver to interpret and understand the data. Data can include both data that stores specific information as well as metadata which is data that describes the data that stores the specific information. Data can be organized in a structured or unstructured format. “Structured data” refers to data within a data structure that is organized according to a predefined format, protocol, or configuration such that the structure may be used to facilitate working with the data. Examples of structured data include, but are not limited to, files, databases, database records, database tables, database schemas, serialized objects, directories, and the like. “Unstructured data” refers to data stored without a particular organization, predefined format, protocol, or configuration. Examples of unstructured data include, but are not limited to, content of a text message, content of an email message, text content of a file, content of a document, and the like. Often the term “data” will be used in connection with one or more adjectives that identify a type or purpose for the data, examples include “user data”, “input data”, “output data”, “sensor data”, “patient data”, “system data”, “map data”, and the like. “Sensor data” refers to any data or information registered by one or more sensors. Examples of sensor data include an amount of current passing through the sensor, an amount of voltage across the sensor, an amount of electrical resistance through the sensor, an amount of strain experienced by the sensor, an acceleration vector, a deceleration vector, an orientation, an orientation angle, a direction, and the like.

As used herein, “implant” refers to a medical device manufactured to replace a missing biological structure, support a damaged biological structure, or enhance an existing biological structure. Often medical implants are man-made devices, but implants can also be natural occurring structures. The surface of implants that contact the body may be made of, or include a biomedical material such as titanium, cobalt chrome, stainless steel, carbon fiber, another metallic alloy, silicone, polymer, Synthetic polyvinyl alcohol (PVA) hydrogels, biomaterials, biocompatible polymers such as PolyEther Ether Ketone (PEEK) or a polylactide polymer (e.g. PLLA) and/or others, or apatite, or any combination of these depending on what is functional and/or economical. Implants can have a variety of configurations and can be wholly, partially, and/or include a number of components that are flexible, semiflexible, pliable, elastic, supple, semi-rigid, or rigid. In some cases, implants contain electronics, e.g. artificial pacemaker and cochlear implants. Some implants are bioactive, such as subcutaneous drug delivery devices in the form of implantable pills or drug-eluting stents. Orthopedic implants may be used to alleviate issues with bones and/or joints of a patient's body. Orthopedic implants can be used to treat bone fractures, osteoarthritis, scoliosis, spinal stenosis, discomfort, and pain. Examples of orthopedic implants include, but are not limited to, a wide variety of pins, rods, screws, anchors, spacers, sutures, all-suture implants, ball all-suture implants, self-locking suture implants, cross-threaded suture implants, plates used to anchor fractured bones while the bones heal or fuse together, and the like. (Search “implant (medicine)” on Wikipedia.com May 26, 2021. CC-BY-SA 3.0 Modified. Accessed Jun. 30, 2021.)

As used herein, a “body” refers to a main or central part of a structure. The body may serve as a structural component to connect, interconnect, surround, enclose, and/or protect one or more other structural components. A body may be made from a variety of materials including, but not limited to, metal, plastic, ceramic, wood, fiberglass, acrylic, carbon, biocompatible materials, biodegradable materials or the like. A body may be formed of any biocompatible materials, including but not limited to biocompatible metals such as Titanium, Titanium alloys, stainless steel alloys, cobalt-chromium steel alloys, nickel-titanium alloys, shape memory alloys such as Nitinol, biocompatible ceramics, and biocompatible polymers such as Polyether ether ketone (PEEK), a polylactide polymer (e.g. PLLA), nylon 12, and/or others. In one embodiment, a body may include a housing or frame, or framework for a larger system, component, structure, or device. A body may include a modifier that identifies a particular function, location, orientation, operation, and/or a particular structure relating to the body. Examples of such modifiers applied to a body, include, but are not limited to, “inferior body,” “superior body,” “lateral body,” “medial body,” and the like.

As used herein, “side” refers to a structure or part of a structure including, but not limited to: one of a longer bounding surfaces or lines of an object especially contrasted with the ends, a line or surface forming a border or face of an object, either surface of a thin object, a bounding line or structure of a geometric figure or shape, and the like. (search “side” on Merriam-Webster.com. Merriam-Webster, 2021. Web. 3 Aug. 2021. Modified.) A side can also refer to a geometric edge of a polygon (two-dimensional shape) and/or a face or surface of a polyhedron (three-dimensional shape). (Search “side” on Wikipedia.com Jul. 21, 2021. CC-BY-SA 3.0 Modified. Accessed Aug. 3, 2021.) Side can also refer to a location on a structure. For example, a side can be a location on a structure at, or near, a furthest position away from a central axis of the structure. As used herein, the term “side” can include one or more modifiers that define and/or orient and/or distinguish the side of an object from others based on based on where and/or how the object is deployed within or in relation to a second object. For example, in the context of an implant for a patient, sides of the implant may be labeled based on where the sides are relative to the patient when the implant is deployed. As one example, an “anterior side” of an implant, instrument, anatomical structure, or other structure refers to a side that is anterior to other sides of the structure in relation to a patient when the structure is deployed in the patient. As another example, in the context of an instrument used with a patient, sides of the instrument may be labeled based on where the sides are when the instrument is being used for its purpose. As one example, a “front side” of an instrument refers to a side that is facing a user of the instrument when the instrument is in use.

As used herein, “bone engagement surface” refers to a surface of an object, instrument, or apparatus, such as an implant that is oriented toward or faces one or more bones of a patient. In one aspect, the bone engagement surface may abut, touch, or contact a surface of a bone. In another aspect, the bone engagement surface or parts of the bone engagement surface may be close to, but not abut, touch, or contact a surface of the bone. In certain aspects, the bone engagement surface can be configured to engage with a surface of one or more bones. Such a bone engagement surface may include projections and recesses that correspond to and match projections and recesses of the one or more bone surfaces.

“Bone engagement member” refers to an apparatus, instrument, structure, device, component, member, system, assembly or module structured, organized, configured, designed, arranged, or engineered to connect, join, link, contact, touch, abut, interface with, couple to, or engage with a bone, a bone part, bony topography (e.g., bone spurs and calcifications), anatomical bone feature, and/or a bone fragment. The connection, coupling, linkage, contact, or engagement may be a mechanical connection or interconnection. A bone engagement member may enable temporary engagement with a bone or bone fragment or permanent engagement with a bone or bone fragment. A bone engagement member may include a bone engagement surface, a bone engagement feature, a body section that supports the bone engagement surface, or the like. In certain embodiments, a bone engagement member may include a bone probe or a joint seeker. In one embodiment, a bone engagement member may include a landmark registration feature. Alternatively, or in addition, a bone engagement member can include a bone attachment feature configured to engage with bone and/or to cooperate with a fastener to engage with bone. A patient-specific bone engagement member is a bone engagement member that includes one or more aspects that are patient-specific. The patient-specific aspects can include, but are not limited to, a surface contour, a contour for a part of a surface, a position for a resection feature, a size, shape and/or configuration of a resection feature, a position, size, shape, and/or number of bone attachment features, or the like.

As used herein, “bone-facing side” refers to a side of an object, structure, instrument, or apparatus, such as an implant or instrument that is oriented toward or faces one or more bones of a patient when a device that includes the bone-facing side is in use. In one aspect, the bone-facing side may abut, touch, or contact a surface of a bone. In another aspect, the bone-facing side or parts of the bone-facing side may be close to, but not abut, touch, or contact a surface of the bone.

“Non-bone-facing side” refers to a side of an object, structure, instrument, or apparatus, such as an implant or instrument that is not oriented toward and/or does not face one or more bones of a patient during use of a device that includes the non-bone-facing side. In certain embodiments, a non-bone-facing side can be a side that is directly opposite a bone-facing side of the same device, object, structure, or apparatus.

“Predetermined Position” refers to a position that is decided, determined, finalized, and/or defined earlier in time. In certain embodiments, a predetermined position is a desired, designed, and/or engineered position of a first object in relation to a second object. Thus, a predetermined position is a planned position for the two objects in relation to each other. In certain embodiments, one or both of the two objects may be moved relative to each other to accomplish the18avigatrmined position and the predetermined position may become the final position. In other embodiments, the two objects may be moved towards the predetermined position but may not reach the exact predetermined position due to some impediment and/or interference or a decision to change the predetermined position to a new position. In certain aspects, a predetermined position may be a position that is decided after a process of recommendation, review, and/or analysis, and final approval such that a position may not become a predetermined position until the process is completed. For example, in a medical patient-specific instrument or technique design process a position may not become the predetermined position until a surgeon or other doctor provides final approval for the position. In certain embodiments, a predetermined position may be indicated, designated, illustrated, defined, and/or explained in a preoperative plan.

As used herein, a “deploy” or “deployment” refers to an act, action, process, system, method, means, or apparatus for inserting an implant or prosthesis into a part, body part, and/or patient. “Deploy” or “deployment” can also refer to an act, action, process, system, method, means, or apparatus for placing something into therapeutic use. A device, system, component, medication, drug, compound, or nutrient may be deployed by a human operator, a mechanical device, an automated system, a computer system or program, a robotic system, or the like.

“Joint” or “Articulation” refers to the connection made between bones in a human or animal body which link the skeletal system to form a functional whole. Joints may be biomechanically classified as a simple joint, a compound joint, or a complex joint. Joints may be classified anatomically into groups such as joints of hand, elbow joints, wrist joints, axillary joints, sternoclavicular joints, vertebral articulations, temporomandibular joints, sacroiliac joints, hip joints, knee joints, articulations of foot, and the like. (Search “joint” on Wikipedia.com Dec. 19, 2021. CC-BY-SA 3.0 Modified. Accessed Jan. 20, 2022.)

“Contour” refers to an outline representing or bounding a shape or form of an object. Contour can also refer to an outside limit of an object, area, or surface of the object. (Search “contour” on wordhippo.com. WordHippo, 2023. Web. Modified. Accessed 13 Jun. 2023.)

“Topographical” refers to the physical distribution of parts, structures, or features on the surface of, or within, an organ or other anatomical structure, or organism. (Search “define topographical” on google.com. Oxford Languages, Copyright 2022. Oxford University Press. Web., Modified. Accessed 15 Feb. 2022.)

“Landmark registration feature” refers to a structure configured to engage, contact, or abut a feature, aspect, attribute, or characteristic of a first object to orient and/or position a second object that includes the landmark registration feature with respect to the first object. A variety of structures can serve as a landmark registration feature. For example, a surface, a probe, a finger, a wing, an arm, an opening, or the like can function as landmark registration features. A landmark registration feature can be of a variety of shapes and thus can include a protrusion, a projection, a tuberosity, a cavity, a void, a divot, a tab, an extension, a hook, a curve, or the like.

“Landmark” refers to a structure on, in, or around a structure that can be used to serve as a reference for positioning, orienting, translating, rotating, or otherwise manipulating a second object or structure. For example, a landmark may include a protrusion, a projection, a tuberosity, a cavity, a void, a divot, a tab, an extension, a hook, a curve, or the like. In the context of bones of a patient, a landmark can include any protuberance, eminence, bony topography, anatomical features, calcifications, void, divot, concave section, sesamoid, bone spur or other feature on, or extending from, a bone of a patient. A landmark refers to any structure of an anatomical structure that is referenced, contacted, engaged with and/or associated with a landmark registration feature. In certain embodiments, a landmark is unique to one patient.

“Bone attachment feature” refers to a structure, feature, component, aspect configured to securely connect, couple, attach, and/or engage a structure, component, object, or body with a bone and/or a bone fragment. Examples of a bone attachment feature, include, but are not limited to, a pin, K-wire, screw, or other fastener alone, or in combination with, a hole, passage, and/or opening.

“Position indicator” refers to any apparatus, structure, device, system, and/or component organized, configured, designed, engineered, and/or arranged to serve as an indicator of a position for one or more things, objects, structures, apparatuses, systems, features, aspects, attributes or the like. Examples of a position indicator include, but are not limited to, a crosshair, cross hairs, a pin, a wire, a fastener, a hole, an opening, a post, a prong, a probe, a needle, an arrow, a marking, or the like. In certain embodiments, an indicator may communicate a position of one structure or component or system in relation to another. A position indicator may indicate a position of one object relative to another, may indicate a relationship between two objects, may indicate a trajectory of one object relative to another, or the like.

As used herein, a “stop” refers to an apparatus, instrument, structure, member, device, component, system, or assembly structured, organized, configured, designed, arranged, or engineered to prevent, limit, impede, stop, or restrict motion or movement and/or operation of the another object, member, structure, component, part, apparatus, system, or assembly.

“Window” refers to an opening and/or a plurality of openings in a body, side, wall, side door, roof, vehicle, system, component, or other structure that allows the passage of electromagnetic radiation including radio ways, x-rays, visible light, light, and the like. A window may also permit passage of sound, gases, fluids, liquids, or other elements. (Search “window” on Wikipedia.com Aug. 31, 2022. Modified. Accessed Sep. 21, 2022.). A window can be opaque, semi-opaque, translucent, radiolucent, or transparent. A window can include a single opening having a single geometric shape or a plurality of openings each of a single geometric shape or combination of a variety of geometric shapes. In certain embodiments, a window may be referred to as a radiolucent window. A radiolucent window may permit passage of some or all radio ways through the window.

“Radiolucent window” refers to a window that permits the passage of radiant energy and electromagnetic radiant energy, in particular, such as x-rays used in an x-ray machine and/or in a fluoroscopy imaging device.

As used herein, a “fastener”, “fixation device”, or “fastener system” refers to any structure configured, designed, or engineered to join two structures. Fasteners may be made of a variety of materials including metal, plastic, composite materials, metal alloys, plastic composites, and the like. Examples of fasteners include, but are not limited to screws, rivets, bolts, nails, snaps, hook and loop, set screws, bone screws, nuts, posts, pins, thumb screws, and the like. Other examples of fasteners include, but are not limited to wires, Kirschner wires (K-wire), anchors, bone anchors, plates, bone plates, intramedullary nails or rods or pins, implants, sutures, soft sutures, soft anchors, tethers, interbody cages, fusion cages, and the like.

In certain embodiments, the term fastener may refer to a fastener system that includes two or more structures configured to combine to serve as a fastener. An example of a fastener system is a rod or shaft having external threads and an opening or bore within another structure having corresponding internal threads configured to engage the external threads of the rod or shaft.

In certain embodiments, the term fastener may be used with an adjective that identifies an object or structure that the fastener may be particularly configured, designed, or engineered to engage, connect to, join, contact, or couple together with one or more other structures of the same or different types. For example, a “bone fastener” may refer to an apparatus for joining or connecting one or more bones, one or more bone portions, soft tissue and a bone or bone portion, hard tissue and a bone or bone portion, an apparatus and a bone or portion of bone, or the like.

In certain embodiments, a fastener may be a temporary fastener. A temporary fastener is configured to engage and serve a fastening function for a relatively short period of time. Typically, a temporary fastener is configured to be used until another procedure or operation is completed and/or until a particular event. In certain embodiments, a user may remove or disengage a temporary fastener. Alternatively, or in addition, another structure, event, or machine may cause the temporary fastener to become disengaged.

As used herein, a “fixator” refers to an apparatus, instrument, structure, device, component, member, system, assembly, or module structured, organized, configured, designed, arranged, or engineered to connect two bones or bone fragments or a single bone or bone fragment and another fixator to position and retain the bone or bone fragments in a desired position and/or orientation. Examples of fixators include both those for external fixation as well as those for internal fixation and include, but are not limited to pins, wires, Kirschner wires, screws, anchors, bone anchors, plates, bone plates, intramedullary nails or rods or pins, implants, interbody cages, fusion cages, and the like.

As used herein, a “fixation” or “fixation device” refers to an apparatus, instrument, structure, device, component, member, system, assembly, step, process, or module structured, organized, configured, designed, arranged, or engineered to connect two structures either permanently or temporarily. The two structures may be one or the other or both of manmade and/or biological tissues, hard tissues such as bones, teeth or the like, soft tissues such as ligament, cartilage, tendon, or the like. In certain embodiments, fixation is used as an adjective to describe a device or component or step in securing two structures such that the structures remain connected to each other in a desired position and/or orientation. Fixation devices can also serve to maintain a desired level of tension, compression, or redistribute load and stresses experienced by the two structures and can serve to reduce relative motion of one part relative to others. Examples of fixation devices are many and include both those for external fixation as well as those for internal fixation and include, but are not limited to pins, wires, Kirschner wires (K-wires), screws, anchors, bone anchors, plates, bone plates, intramedullary nails or rods or pins, implants, interbody cages, fusion cages, and the like.

As used herein, an “anchor” refers to an apparatus, instrument, structure, member, part, device, component, system, or assembly structured, organized, configured, designed, arranged, or engineered to secure, retain, stop, and/or hold, an object to or at a fixed point, position, or location. An anchor may be coupled and/or connected to a flexible member such as a tether, chain, rope, wire, thread, suture, suture tape, or other like object. Alternatively, or in addition, an anchor may also be coupled, connected, and/or joined to a rigid object or structure. In certain embodiments, an anchor can be a fixation device. Said another way, a fixation device can function as an anchor. For example, an anchor pin is a pin, fastener, or K-wire that cooperates with a rigid structure to provide an anchor.

“Connector” refers to any structure configured, engineered, designed, adapted, and/or arranged to connect one structure, component, element, or apparatus to another structure, component, element, or apparatus. A connector can be rigid, pliable, elastic, flexible, and/or semiflexible. Examples of a connector include but are not limited to any fastener.

As used herein, a “sleeve” refers to structure that is narrow and longer longitudinally than the structure is wide. In certain embodiments, a sleeve serves to surround, enclose, wrap, and/or contain something else. In certain embodiments, a sleeve may surround, enclose, wrap, and/or contain a passage or void. (Search “sleeve” on wordhippo.com. WordHippo, 2021. Web. Accessed 15 Nov. 2021. Modified.) In certain embodiments, the term sleeve may be preceded by an adjective that identifies the structure, implement, component or instrument that may be used with, inserted into or associated with the sleeve. For example, a “pin sleeve” may be configured to accept a pin or wire such as a K-wire, a “drive sleeve” may be configured to accept a drill or drill bit, a “fixation member sleeve” may be configured to accept a fastener or fixation member.

As used herein, a “long bone” refers to a bone of a patient having a length greater than a width of the bone. Long bone is one of five types of bones: long, short, flat, irregular and sesamoid. Long bones, especially the femur and tibia, can be subjected to most of the load during daily activities. Long bones grow primarily by elongation of the diaphysis, with an epiphysis at each end of the growing bone. The ends of epiphyses are covered with hyaline cartilage (“articular cartilage”). The longitudinal growth of long bones is a result of endochondral ossification at the epiphyseal plate. The long bone category type includes the femur, tibia, and fibula of the legs; the humerus, radius, and ulna of the arms; metacarpals and metatarsals of the hands and feet, the phalanges of the fingers and toes, and the clavicles or collar bones in humans or other patients. The outside of the long bone consists of a layer of connective tissue called the periosteum. Additionally, the outer shell of the long bone is compact bone, then a deeper layer of cancellous bone (spongy bone) which includes a medullary cavity that includes bone marrow. (Search “long bone” on Wikipedia.com May 14, 2021. CC-BY-SA 3.0 Modified. Accessed Jul. 26, 2021.)

“Talar dome” refers to part of a talus bone. Specifically, the talar dome refers to the superior convex surface and/or area of the talus. The talar dome may also be referred to as a trochlea of the talus. The talar dome is part of the talus body.

“Bone fragment” or “fragment” refers to a part of a bone that is normally part of another bone of a patient. A bone fragment may be separate from another bone of a patient due to a deformity or trauma. In one aspect, the bone the bone fragment is normally connected or joined with is referred to as a parent bone.

As used herein, “manufacturing tool” or “fabrication tool” refers to a manufacturing or fabrication process, tool, system, or apparatus which creates an object, device, apparatus, feature, or component using one or more source materials. A manufacturing tool or fabrication tool can use a variety of manufacturing processes, including but not limited to additive manufacturing, subtractive manufacturing, forging, casting, and the like. The manufacturing tool can use a variety of materials including polymers, thermoplastics, metals, biocompatible materials, biodegradable materials, ceramics, biochemicals, and the like. A manufacturing tool may be operated manually by an operator, automatically using a computer numerical controller (CNC), or a combination of these techniques.

As used herein, “osteotomy procedure” or “surgical osteotomy” or “osteotomy” refers to a surgical operation in which one or more bones are cut to shorten or lengthen them or to change their alignment. The procedure can include removing one or more portions of bone and/or adding one or more portions of bone or bone substitutes. (Search “osteotomy” on Wikipedia.com Feb. 3, 22, 2021. CC-BY-SA 3.0 Modified. Accessed Feb. 15, 2022.)

As used herein, “patient-specific osteotomy procedure” refers to an osteotomy procedure that has been adjusted, tailored, modified, or configured to specifically address the anatomy, physiology, condition, abnormalities, needs, or desires of a particular patient. In certain aspects, one patient-specific osteotomy procedure may be useable in connection with only one patient. In other aspects, one patient-specific osteotomy procedure may be useable with a number of patients having a particular class of characteristics. In certain aspects, a patient-specific osteotomy procedure may refer to a non-patient-specific osteotomy procedure that includes one or more patient-specific implants and/or instrumentation. In another aspects, a patient-specific osteotomy procedure may refer to a patient-specific osteotomy procedure that includes one or more patient-specific implants, patient-specific surgical steps, and/or patient-specific instrumentation.

“Register” or “Registration” refers to an act of aligning, mating, contacting, engaging, or coupling one or more parts and/or surfaces of one object in relation to one or more parts and/or surfaces of another object. Often, the one or more parts and/or surfaces of one object include protrusions and/or depressions that are the inverse or mirror configuration of protrusions and/or depressions of one or more parts and/or surfaces of the other object.

“Remediation procedure” refers to any designed or performed for the purpose of remediating a condition of a patient and/or a condition of one or more parts of a body of a patient.

“Wedge osteotomy” refers to an osteotomy procedure in which one or more wedges are used as part of the procedure. Generally, wedge osteotomies can be of one of two types, open wedge and closing wedge. The type of osteotomy refers to how the procedure changes the relation between two parts of a bone involved in the osteotomy. In an open wedge osteotomy, a wedge of bone or graft or other material is inserted in between two parts of a bone. Consequently, a wedge shape is “opened” in the bone. In a close/closed wedge osteotomy or closing wedge osteotomy, a wedge of bone is removed from a bone. Consequently, a wedge shape formed in the bone is “closed.”

As used herein, an “opening” refers to a gap, a hole, an aperture, a port, a portal, a slit, a space or recess in a structure, a void in a structure, or the like. In certain embodiments, an opening can refer to a structure configured specifically for receiving something and/or for allowing access. In certain embodiments, an opening can pass through a structure. In such embodiments, the opening can be referred to as a window. In other embodiments, an opening can exist within a structure but not pass through the structure. In other embodiments, an opening can initiate on a surface or at an edge or at a side of a structure and extend into the structure for a distance, but not pass through or extend to another side or edge of the structure. In other embodiments, an opening can initiate on a surface or at an edge or at a side of a structure and extend into the structure until the opening extends through or extends to another side or edge of the structure. An opening can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “opening” can include one or more modifiers that define specific types of “openings” based on the purpose, function, operation, position, or location of the “opening.” As one example, a “fastener opening” refers to an “opening” adapted, configured, designed, or engineered to accept or accommodate a “fastener.”

“Hole” refers to a gap, an opening, an aperture, a port, a portal, a space or recess in a structure, a void in a structure, or the like. In certain embodiments, a hole can refer to a structure configured specifically for receiving something and/or for allowing access. In certain embodiments, a hole can pass through a structure. In other embodiments, an opening can exist within a structure but not pass through the structure. A hole can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “hole” can include one or more modifiers that define specific types of “holes” based on the purpose, function, operation, position, or location of the “hole.” As one example, a “fastener hole” refers to an “hole” adapted, configured, designed, or engineered to accept or accommodate a “fastener.” A “blind hole” is a hole with an opening on one side that does not extend all the way through a structure. In certain embodiments, a hole, including a blind hole, has a circular longitudinal cross-section. Alternatively, or in addition, a hole can have a cross-section of a variety of geometric shapes include a circle, an oval, a square, a rectangle, a slot with rounded ends, a triangle, or the like.

As used herein, “end” refers to a part or structure of an area or span that lies at the boundary or edge. An end can also refer to a point that marks the extent of something and/or a point where something ceases to exist. An end can also refer to an extreme or last part lengthwise of a structure or surface. (search “end” on Merriam-Webster.com. Merriam-Webster, 2021. Web. 4 Aug. 2021. Modified.)

“Trajectory guide” or “trajectory indicator” or “targeting guide” refers to any structure, apparatus, surface, device, system, feature, or aspect configured to indicate, identify, guide, place, position, or otherwise assist in marking or deploying a fastener or other structure along a desired trajectory for one or more subsequent steps in a procedure.

As used herein, “anatomic data” refers to data identified, used, collected, gathered, and/or generated in connection with an anatomy of a human or animal. Examples of anatomic data may include location data for structures, both independent, and those connected to other structures within a coordinate system. Anatomic data may also include data that labels or identifies one or more anatomical structures. Anatomic data can include volumetric data, material composition data, and/or the like. Anatomic data can be generated based on medical imaging data or measurements using a variety of instruments including monitors and/or sensors. Anatomic data can be gathered, measured, or collected from anatomical models and/or can be used to generate, manipulate, or modify anatomical models.

A bone model or anatomic model of a patient's body or body part(s) may be generated by computing devices that analyze medical imaging images. Structures of a patient's body can be determined using a process called segmentation.

“Set” refers to a collection of objects. A set can have zero or more objects in the collection. Generally, a set includes one or more objects in the collection.

“Trajectory guide” or “trajectory indicator” or “targeting guide” refers to any structure, apparatus, surface, device, system, feature, or aspect configured to indicate, identify, guide, place, position, or otherwise assist in marking or deploying a fastener or other structure along a desired trajectory for one or more subsequent steps in a procedure.

“Trajectory” refers to a path a body travels or a path configured for a body to travel through space. (Search “trajectory” on wordhippo.com. WordHippo, 2023. Web. Modified. Accessed 13 Jun. 2023.)

As used herein, a “guard” refers to an apparatus, instrument, structure, member, device, component, system, or assembly structured, organized, configured, designed, arranged, or engineered to prevent, limit, impede, stop, or restrict motion, action, or movement and/or operation of the another object, member, structure, component, part, apparatus, system, or assembly beyond a certain parameter such as a boundary. Said another way, a “guard” refers to an apparatus, instrument, structure, member, device, component, system, or assembly structured, organized, configured, designed, arranged, or engineered to retain, maintain, hold, keep, or restrict motion, action, or movement and/or operation of the another object, member, structure, component, part, apparatus, system, or assembly within or at one or more parameters such as a boundary.

As used herein, “artificial intelligence” refers to intelligence demonstrated by machines, unlike the natural intelligence displayed by humans and animals, which involves consciousness and emotionality. The distinction between artificial intelligence and natural intelligence categories is often revealed by the acronym chosen. ‘Strong’ AI is usually labelled as artificial general intelligence (AGI) while attempts to emulate ‘natural’ intelligence have been called artificial biological intelligence (ABI). Leading AI textbooks define the field as the study of “intelligent agents”: any device that perceives its environment and takes actions that maximize its chance of achieving its goals. The term “artificial intelligence” can also be used to describe machines that mimic “cognitive” functions that humans associate with the human mind, such as “learning” and “problem solving”. (Search “artificial intelligence” on Wikipedia.com Jun. 25, 2021. CC-BY-SA 3.0 Modified. Accessed Jun. 25, 2021.)

As used herein, “segmentation” or “image segmentation” refers to the process of partitioning an image into different meaningful segments. These segments may correspond to different tissue classes, organs, pathologies, bones, or other biologically relevant structures. Medical image segmentation accommodates imaging ambiguities such as low contrast, noise, and other imaging ambiguities.

Certain computer vision techniques can be used or adapted for image segmentation. For example, the techniques and or algorithms for segmentation may include, but are not limited to: Atlas-Based Segmentation: For many applications, a clinical expert can manually label several images; segmenting unseen images is a matter of extrapolating from these manually labeled training images. Methods of this style are typically referred to as atlas-based segmentation methods. Parametric atlas methods typically combine these training images into a single atlas image, while nonparametric atlas methods typically use all of the training images separately. Atlas-based methods usually require the use of image registration in order to align the atlas image or images to a new, unseen image.

Image registration is a process of correctly aligning images; Shape-Based Segmentation: Many methods parametrize a template shape for a given structure, often relying on control points along the boundary. The entire shape is then deformed to match a new image. Two of the most common shape-based techniques are Active Shape Models and Active Appearance Models; Image-Based Segmentation: Some methods initiate a template and refine its shape according to the image data while minimizing integral error measures, like the Active contour model and its variations; Interactive Segmentation: Interactive methods are useful when clinicians can provide some information, such as a seed region or rough outline of the region to segment. An algorithm can then iteratively refine such a segmentation, with or without guidance from the clinician. Manual segmentation, using tools such as a paint brush to explicitly define the tissue class of each pixel, remains the gold standard for many imaging applications. Recently, principles from feedback control theory have been incorporated into segmentation, which give the user much greater flexibility and allow for the automatic correction of errors; Subjective surface Segmentation: This method is based on the idea of evolution of segmentation function which is governed by an advection-diffusion model. To segment an object, a segmentation seed is needed (that is the starting point that determines the approximate position of the object in the image). Consequently, an initial segmentation function is constructed. With the subjective surface method, the position of the seed is the main factor determining the form of this segmentation function; and Hybrid segmentation which is based on combination of methods. (Search “medical image computing” on Wikipedia.com Jun. 24, 2021. CC-BY-SA 3.0 Modified. Accessed Jun. 24, 2021.)

As used herein, “medical imaging” refers to a technique and process of imaging the interior or exterior of a body for clinical analysis and medical intervention, as well as visual representation of the function of some organs or tissues (physiology). Medical imaging seeks to reveal internal structures hidden by the skin and bones, as well as to diagnose and treat disease. Medical imaging may be used to establish a database of normal anatomy and physiology to make possible identification of abnormalities.

Medical imaging in its widest sense, is part of biological imaging and incorporates radiology, which uses the imaging technologies of X-ray radiography, magnetic resonance imaging, ultrasound, endoscopy, elastography, tactile imaging, thermography, medical photography, nuclear medicine functional imaging techniques as positron emission tomography (PET) and single-photon emission computed tomography (SPECT). Another form of X-ray radiography includes computerized tomography (CT) scans in which a computer may control the position of the X-ray sources and detectors. Magnetic Resonance Imaging (MRI) is another medical imaging technology. Fluoroscopy is an imaging technique that uses X-rays to obtain real-time moving images of the interior of an object. In its primary application of medical imaging, a fluoroscope allows a physician to see the internal structure and function of a patient, so that the pumping action of the heart or the motion of swallowing, for example, can be watched. This is useful for both diagnosis and therapy and occurs in general radiology, interventional radiology, and image-guided surgery. (Search “medical imaging” on Wikipedia.com Jul. 14, 2021. CC-BY-SA 3.0 Modified. Accessed Sep. 1, 2021.)

Data analyzed, generated, manipulated, interpolated, collected, stored, reviewed, and/or modified in connection with medical imaging or medical image processing can be referred to herein as medical imaging data or medical image data.

Measurement and recording techniques that are not primarily designed to produce images, such as electroencephalography (EEG), magnetoencephalography (MEG), electrocardiography (ECG), and others, represent other technologies that produce data susceptible to representation as a parameter graph vs. time or maps that contain data about the measurement locations. In certain embodiments bone imaging includes devices that scan and gather bone density anatomic data. These technologies may be considered forms of medical imaging in certain disciplines. (Search “medical imaging” on Wikipedia.com Jun. 16, 2021. CC-BY-SA 3.0 Modified. Accessed Jun. 23, 2021.)

As used herein, As used herein, “patient imaging data” refers to data identified, used, collected, gathered, and/or generated in connection with medical imaging for a particular patient. Patient imaging data is one type of medical imaging data. Patient imaging data can be shared between users, systems, patients, and professionals using a common data format referred to as Digital Imaging and Communications in Medicine (DICOM) data. DICOM data is a standard format for storing, viewing, retrieving, and sharing medical images.

As used herein, “medical image computing” or “medical image processing” refers to systems, software, hardware, components, and/or apparatus that involve and combine the fields of computer science, information engineering, electrical engineering, physics, mathematics and medicine. Medical image computing develops computational and mathematical methods for working with medical images and their use for biomedical research and clinical care. One goal for medical image computing is to extract clinically relevant information or knowledge from medical images. While closely related to the field of medical imaging, medical image computing focuses on the computational analysis of the images, not their acquisition. The methods can be grouped into several broad categories: image segmentation, image registration, image-based physiological modeling, and others. (Search “medical image computing” on Wikipedia.com Jun. 24, 2021. CC-BY-SA 3.0 Modified. Accessed Jun. 24, 2021.) Medical image computing may include one or more processors or controllers on one or more computing devices. Such processors or controllers may be referred to herein as medical image processors. Medical imaging and medical image computing together can provide systems and methods to image, quantify and fuse both structural and functional information about a patient in vivo. These two technologies include the transformation of computational models to represent specific subjects/patients, thus paving the way for personalized computational models. Individualization of generic computational models through imaging can be realized in three complementary directions: definition of the subject-specific computational domain (anatomy) and related subdomains (tissue types); definition of boundary and initial conditions from (dynamic and/or functional) imaging; and characterization of structural and functional tissue properties. Medical imaging and medical image computing enable the translation of models to the clinical setting with both diagnostic and therapeutic applications. (Id.) In certain embodiments, medical image computing can be used to generate a bone model, a patient-specific model, and/or a patent specific instrument from medical imaging and/or medical imaging data.

As used herein, “model” refers to an informative representation of an object, person or system. Representational models can be broadly divided into the concrete (e.g. physical form) and the abstract (e.g. behavioral patterns, especially as expressed in mathematical form). In abstract form, certain models may be based on data used in a computer system or software program to represent the model. Such models can be referred to as computer models. Computer models can be used to display the model, modify the model, print the model (either on a 2D medium or using a 3D printer or additive manufacturing technology). The printed physical form of the model can be referred to as a 3D model. Computer models can also be used in environments with models of other objects, people, or systems. Computer models can also be used to generate simulations, display in virtual environment systems, display in augmented reality systems, or the like. Computer models can be used in Computer Aided Design (CAD) and/or Computer Aided Manufacturing (CAM) systems. Certain models may be identified with an adjective that identifies the object, person, or system the model represents. For example, a “bone” model is a model of a bone, and a “heart” model is a model of a heart. (Search “model” on Wikipedia.com Jun. 13, 2021. CC-BY-SA 3.0 Modified. Accessed Jun. 23, 2021.)

A “bone model” or “anatomic model” refers to a model of a bone of a person. The bone model may model a single bone or a plurality of bones. The modeled bone and/or bones may be positioned in standard anatomical form and/or may be positioned relative to other bones (e.g., models of bones) of a person such that the positions of the bones in the bone model are the same or substantially the same as corresponding bones of a person, such as a patient.

“Position” refers to a place or location. (Search “position” on wordhippo.com. WordHippo, 2024. Web. Accessed 8 Jan. 2024.) A position may be defined in a virtual environment such as in a model or set of models defined by and presented by a computing device. In addition, a position may be a place or location in a tangible physical environment such as in a space, on land, within or on a system, assembly, component, a patient, or other structure.

“Original position” refers to a position before any actions are taken to change a position of a structure, object, device, apparatus, component, or system. An original position may be defined in a virtual environment such as in a model or set of models defined by and presented by a computing device. Alternatively, or in addition, an original position may be a position in, on, or part of a tangible physical object, such as bones of a foot in a patient. In certain embodiments, an original position is a deformed position. An original position can be contrasted with a predetermined position which may be a position planned to implement a correction, correct a structure's position from a deformed position to a corrected position.

A “deformed position” refers to an anatomical structure that is positioned to form, include, or is at least part of a deformity. A “corrected position” refers to an anatomical structure positioned to remediate, correct, eliminate, and/or overcome a deformity. A predetermined position can be a corrected position.

“Feedback” refers to a reactionary response to an action, a product, service, or task. (Search “feedback” on wordhippo.com. WordHippo, 2023. Web. Modified. Accessed 28 Aug. 2023.)

As used herein, “additive manufacturing” refers to a manufacturing process in which materials are joined together in a process that repeatedly builds one layer on top of another to generate a three-dimensional structure or object. Additive manufacturing may also be referred to using different terms including additive processes, additive fabrication, additive techniques, additive layer manufacturing, layer manufacturing, freeform fabrication, ASTM F2792 (American Society for Testing and Materials), and 3D printing. Additive manufacturing can build the three-dimensional structure or object using computer-controlled equipment that applies successive layers of the material(s) based on a three-dimensional model that may be defined using Computer Aided Design (CAD) software. Additive manufacturing can use a variety of materials including polymers, thermoplastics, metals, ceramics, biochemicals, and the like. Additive manufacturing may provide unique benefits, as an implant together with the pores and/or lattices can be directly manufactured (without the need to generate molds, tool paths, perform any milling, and/or other manufacturing steps).

“Palpable feedback” refers to a type of feedback that can be felt. In one embodiment, palpable feedback can refer to feedback that is readily noticeable, tangible, or easily felt or perceived. During certain medical procedures, such as an osteotomy, palpable feedback can include the tactile sensations experienced by the healthcare provider as they move bones or bone fragments. (© ChatGPT 3.5 Version, Modified, accessed chat.openai.com/chat Feb. 2, 2024). Often palpable feedback is feedback a user, such as a surgeon, feels as they perform one or more steps or actions in a surgical procedure.

“Three-dimensional surface” refers to a surface defined by a collection of points that have three coordinates (x, y, z), where each point represents a location in space. In a medical context, a three-dimensional surface can include a surface of an implant or instrument that is customized for a particular purpose. In certain embodiments, a three-dimensional surface may be customized to fit anatomy of a patient or to accommodate handling by a user (e.g., a handle). (© ChatGPT 3.5 Version, Modified, accessed chat.openai.com/chat Feb. 2, 2024). In certain embodiments, of the present disclosure, a three-dimensional surface may be a surface on a side of an instrument that is specifically configured or customized to fit or match anatomy of a patient.

“Repository” refers to any data source or dataset that includes data or content. In one embodiment, a repository resides on a computing device. In another embodiment, a repository resides on a remote computing or remote storage device. A repository may comprise a file, a folder, a directory, a set of files, a set of folders, a set of directories, a database, an application, a software application, content of a text, content of an email, content of a calendar entry, and the like. A repository, in one embodiment, comprises unstructured data. A repository, in one embodiment, comprises structured data such as a table, an array, a queue, a look up table, a hash table, a heap, a stack, or the like. A repository may store data in any format Including binary, text, encrypted, unencrypted, a proprietary format, or the like.

As used herein, “bevel” refers to an edge of a structure that is not perpendicular to the faces of the piece, the edge has a slope or slant or angled profile and can refer to a sloped surface. Often a cutting tool such as a blade or tooth can have a beveled edge that facilitates the cutting edge in cutting into a target material. “bevel” and “chamfer” can be used interchangeably herein. (Search “bevel” on Wikipedia.com May 17, 2021. CC-BY-SA 3.0 Modified. Accessed Aug. 4, 2021; search “bevel” on Merriam-Webster.com. Merriam-Webster, 2021. Web. Accessed 4 Aug. 2021. Modified; search “bevel” on wordhippo.com. WordHippo, 2021. Web. Accessed 4 Aug. 2021. Modified.)

As used herein, “registration” or “image registration” refers to a method, process, module, component, apparatus, and/or system that seeks to achieve precision in the alignment of two images. As used here, “image” may refer to either or both an image of a structure or object and another image or a model (e.g., a computer-based model or a physical model, in either two dimensions or three dimensions). In the simplest case of image registration, two images are aligned. One image may serve as the target image and the other as a source image; the source image is transformed, positioned, realigned, and/or modified to match the target image. An optimization procedure may be applied that updates the transformation of the source image based on a similarity value that evaluates the current quality of the alignment. An iterative procedure of optimization may be repeated until a (local) optimum is found. An example is the registration of CT and PET images to combine structural and metabolic information. Image registration can be used in a variety of medical applications: Studying temporal changes; Longitudinal studies may acquire images over several months or years to study long-term processes, such as disease progression. Time series correspond to images acquired within the same session (seconds or minutes). Time series images can be used to study cognitive processes, heart deformations and respiration; Combining complementary information from different imaging modalities. One example may be the fusion of anatomical and functional information.

Since the size and shape of structures vary across modalities, evaluating the alignment quality can be more challenging. Thus, similarity measures such as mutual information may be used; Characterizing a population of subjects. In contrast to intra-subject registration, a one-to-one mapping may not exist between subjects, depending on the structural variability of the organ of interest. Inter-subject registration may be used for atlas construction in computational anatomy. Here, the 34avigative may be to statistically model the anatomy of organs across subjects; Computer-assisted surgery: in computer-assisted surgery pre-operative images such as CT or MRI may be registered to intra-operative images or tracking systems to facilitate image guidance or navigation. There may be several considerations made when performing image registration: The transformation model. Common choices are rigid, affine, and deformable transformation models. B-spline and thin plate spline models are commonly used for parameterized transformation fields. Non-parametric or dense deformation fields carry a displacement vector at every grid location; this may use additional regularization constraints. A specific class of deformation fields are diffeomorphisms, which are invertible transformations with a smooth inverse; The similarity metric. A distance or similarity function is used to quantify the registration quality. This similarity can be calculated either on the original images or on features extracted from the images. Common similarity measures are sum of squared distances (SSD), correlation coefficient, and mutual information. The choice of similarity measure depends on whether the images are from the same modality; the acquisition noise can also play a role in this decision. For example, SSD may be the optimal similarity measure for images of the same modality with Gaussian noise. However, the image statistics in ultrasound may be significantly different from Gaussian noise, leading to the introduction of ultrasound specific similarity measures.

Multi-modal registration may use a more sophisticated similarity measure; alternatively, a different image representation can be used, such as structural representations or registering adjacent anatomy; The optimization procedure. Either continuous or discrete optimization is performed. For continuous optimization, gradient-based optimization techniques are applied to improve the convergence speed. (Search “medical image computing” on Wikipedia.com Jun. 24, 2021. CC-BY-SA 3.0 Modified. Accessed Jun. 25, 2021.)

As used herein, a “resection” refers to a method, procedure, or step that removes tissue from another anatomical structure or body. A resection is typically performed by a surgeon on a part of a body of a patient. (Search “surgery” on Wikipedia.com May 26, 2021. CC-BY-SA 3.0 Modified. Accessed May 26, 2021.) In certain embodiments, a resection may remove little or no tissue and may in such circumstances also be referred to as an incision or a dissection. Resection may be used as a noun or a verb. In the verb form, the term is “resect” and refers to an act of performing, or doing, a resection. Past tense of the verb resect is resected.

“Resection feature” refers to any feature configured, designed, engineered and/or intended to facilitate resection. Examples of a resection guide feature include but are not limited to, a slot, a cut channel, a cut slot, a pivoting cut guide, a pivoting resection guide, an opening, a straight slot, an angled slot, a curved slot, or the like.

“Patient-matched” refers to a feature, aspect, attribute, characteristic, instrument, and/or device that is selected from a set of predetermined, predefined, precalculated, preconfigured, prearranged, and/or pre-fabricated structures, apparatuses, devices, instruments or devices to satisfactorily service a user based on a set of characteristics, such as size of an anatomical structure, deformity, fracture, laceration, opening, angles for certain landmarks, angles for a deformity, type of deformity, size of the bone, and the like. In certain embodiments, patient-matched is different from patient-specific.

“Bone condition” refers to any of a variety of conditions of bones of a patient. Generally, a bone condition refers to an orientation, position, and/or alignment of one or more bones of the patient relative to other anatomical structures of the body of the patient. Bone conditions may be caused by or result from deformities, misalignment, malrotation, fractures, joint failure, and/or the like. A bone condition includes, but is not limited to, any angular deformities of one or more bone segments in either the lower or upper extremities (for example, tibial deformities, calcaneal deformities, femoral deformities, and radial deformities). Alternatively, or in addition, “bone condition” can refer to the structural makeup and configuration of one or more bones of a patient. Thus bone condition may refer to a state or condition of regions, a thickness of a cortex, bone density, a thickness and/or porosity of internal regions (e.g. whether it is calcaneus or solid) of the bone or parts of the bone such as a head, a base, a shaft, a protuberance, a process, a lamina, a foramen, and the like of a bone, along the metaphyseal region, epiphysis region, and/or a diaphyseal region. “Malrotation” refers to a condition in which a part, typically a part of a patient's body has rotated from a normal position to an unnormal or uncommon position.

As used herein, a “guide” refers to a part, component, member, or structure designed, adapted, configured, or engineered to guide or direct one or more other parts, components, or structures. A guide may be part of, integrated with, connected to, attachable to, or coupled to, another structure, device, or instrument. In one embodiment, a guide may include a modifier that identifies a particular function, location, orientation, operation, type, and/or a particular structure of the guide. Examples of such modifiers applied to a guide, include, but are not limited to, “pin guide” that guides or directs one or more pins, a “cutting guide” that guides or directs the making or one or more cuts, a placement, deployment, or insertion guide that guides or directs the placement, positioning, orientation, deployment, installation, or insertion of a fastener and/or implant, a “cross fixation guide” that guides deployment of a fastener or fixation member, an “alignment guide” that guides the alignment of two or more objects or structures, a “resection guide” that serves to guide resection of soft or hard tissue, such as in an osteotomy, a “reduction guide” can serve to guide reduction of one or more bone segments or fragments, an “placement guide” that serves to identify how an object can be placed in relation to another object or structure, and the like. Furthermore, guides may include modifiers applied due to the procedure or location within a patient for which the guide is to be used. For example, where a guide is used at a joint, the guide may be referred to herein as an “arthrodesis guide”.

“Guide pin” refers to a pin, structure, or a type of fastener that can be used to guide an instrument or implant as part of a method, process, or procedure, such as a surgical technique. In certain aspects, a guide pin may be designed for temporary use until subsequent steps in a method, process, or procedure. Examples of a guide pin include, but are not limited to, a pin, a K-wire, and the like.

“Straight cut” is a type of cut that may be used for a surgical procedure. Generally, a straight cut is a cut in tissue (soft tissue or hard tissue) that is perpendicular to a surface where the cut is made and extends within and/or through the tissue along a straight line. Advantageously, straight cuts may be easier for a surgeon to perform than an angled cut or a curved cut. In certain embodiments, a straight cut is made with a guide (e.g., cut guide, resection guide, and/or resection feature). Alternatively, or in addition, a user may make a straight cut free-hand (without the aid of a guide, instrument, or instrumentation).

As used herein, “feature” refers to a distinctive attribute or aspect of something. (Search “feature” on google.com. Oxford Languages, 2021. Web. 20 Apr. 2021.) A feature may include one or more apparatuses, structures, objects, systems, sub-systems, devices, or the like. A feature may include a modifier that identifies a particular function or operation and/or a particular structure relating to the feature. Examples of such modifiers applied to a feature, include, but are not limited to, “attachment feature,” “securing feature,” “placement feature,” “protruding feature,” “engagement feature,” “disengagement feature,” “resection feature”, “guide feature”, and the like.

“Engagement feature” or “Engagement member” refers to an apparatus, instrument, structure, device, component, member, system, assembly or module structured, organized, configured, designed, arranged, or engineered to connect, join, link, couple to, or engage with another object, apparatus, instrument, structure, device, component, member, system, assembly or module either permanently or temporarily. The connection, coupling, linkage, or engagement may be a mechanical connection or interconnection.

“Cortical bone” refers to a type of bone tissue. Cortical bone is a type of bone tissue typically found between an external surface of a bone and an interior area of the bone. Cortical bone is more dense and typically stronger structurally than other types of bone tissue. “Cortical surface” refers to a surface of cortical bone.

“Transosseous placement feature” refers to a placement feature that extends through one or more bones and that enables, or facilitates, placement of another device, apparatus, or instrument.

“Patient-specific” refers to an attribute, aspect, feature, characteristic, function, structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient and/or surgeon serving the particular patient. In one aspect, a patient specific aspect or feature is unique to a single patient and may include features unique to the patient such as a number of cut channels, a number of bone attachment features, a number of bone engagement surfaces, a number of resection features, a depth of one or more cutting channels, an angle for one or more resection channels, a surface contour, component position, component orientation, and/or other features.

“Patient specific feature” refers to a feature, function, structure, device, guide, tool, instrument, apparatus, member, component, system, assembly, module, or subsystem that is adjusted, tailored, modified, organized, configured, designed, arranged, engineered, and/or fabricated to specifically address the anatomy, physiology, condition, abnormalities, needs, or desires of a particular patient or surgeon serving the particular patient. In one aspect, a patient specific feature is unique to a single patient and may include features unique to the patient such as a number of cut channels, a number of bone attachment features, a number of bone engagement surfaces, a number of resection features, a depth of one or more cutting channels, an angle for one or more resection channels, a surface contour, component position, component orientation, and/or other features. “Medial resection guide” refers to a resection guide designed, engineered, fabricated, or intended for use with, one, in, or about a medial part, section, surface, portion, or aspect of an anatomical structure such as a bone, digit, limb, or other anatomical structure for one or more steps of a resection procedure. “Lateral resection guide” refers to a resection guide designed, engineered, fabricated, or intended for use with, one, in, or about a lateral part, section, surface, portion, or aspect of an anatomical structure such as a bone, digit, limb, or other anatomical structure for one or more steps of a resection procedure.

“Bone fragment” refers to a part of a bone that is normally part of another bone of a patient. A bone fragment may be separate from another bone of a patient due to a deformity or trauma. In one aspect, the bone the bone fragment is normally connected or joined with is referred to as a parent bone.

“Orientation” refers to a direction, angle, position, condition, state, or configuration of a first object, component, part, apparatus, system, or assembly relative to another object, component, part, apparatus, system, assembly, reference point, reference axis, or reference plane.

“Longitudinal axis” refers to an axis of a structure, device, object, apparatus, or part thereof that extends from one end of a longest dimension to an opposite end. Typically, a longitudinal axis passes through a center of the structure, device, object, apparatus, or part thereof along the longitudinal axis. The center point used for the longitudinal axis may be a geometric center point and/or a mass center point.

Those of skill in the art will appreciate that a resection feature may take a variety of forms and may include a single feature or one or more features that together form the resection feature. In certain embodiments, the resection feature may take the form of one or more slots. Alternatively, or in addition, a resection feature may be referenced using other names including, but not limited to, channel, cut channels, and the like.

“Cutting tool” refers to any tool that can be used to cut or resect another object. In particular, a cutting tool can refer to a manual or power tool for cutting or resecting tissue of a patient. Examples of cutting tools include, but are not limited to, a burr, an oscillating saw, a reciprocating saw, a grater saw, a drill, a mill, a side-cutting burr, or the like.

“Revised model” refers to a model that has been changed from an original condition to an altered or changed or revised condition. Generally, an original model is used to create the revised model. Alternatively, a revised model can be generated from scratch. Often the original model exists in a digital form on a computer. Such models can be referred to as CAD models.

“Corrected model” refers to a model in which an object, structure, and/or subject of the model has been changed from a deformed or incorrect configuration to a corrected or revised configuration. A corrected model can be generated from scratch or can be generated by revising an existing model and/or merging two or more models. As one example, an original model may represent one or more bones of a foot. The modeled bones of the foot may be subject to a bone condition and therefore have a deformity. A corrected model can be created or formed from the original model by revising or changing one or more aspects of the original model such that the modeled bones reflect a corrected orientation and or configuration for the modeled bones.

As used herein, “edge” refers to a structure, boundary, or line where an object, surface, or area begins or ends. An edge can also refer to a boundary or perimeter between two structures, objects, or surfaces. An edge can also refer to a narrow part adjacent to a border. (search “edge” on Merriam-Webster.com. Merriam-Webster, 2021. Web. 3 Aug. 2021. Modified.) In certain embodiments, an edge can be a one dimensional or a two-dimensional structure that joins two adjacent structures or surfaces. Furthermore, an edge may be at a perimeter of an object or within a perimeter or boundary of an object.

“Boundary” refers to a structure, line, or area where an object, surface, line, area, or operation is or is expected to begin and/or end. A boundary can be similar to a border.

“Dimension” refers to a measure of spatial extent in a particular direction, such as height, width or breadth, span, thickness, or depth. (Search “dimension” on wordhippo.com. WordHippo, 2023. Web. Modified. Accessed Feb. 17, 2023.)

The present disclosure discloses methods, systems, and/or apparatuses for harvesting bone grafts. In one embodiment, the present disclosure describes how to provide a harvesting guide. In certain embodiments, the present disclosure describes how to provide a patient-specific harvesting guide. In certain embodiments, the harvesting guide may be used to harvest an autograft bone graft. For example, the harvesting guide may be used to harvest an autograft bone graft from a calcaneus of a patient. Advantageously, the harvesting guide may be configured to harvest an autograft bone graft of a custom patient-specific size (length/depth/span/thickness, width, height) that has required dimensions for an opening formed by one or more osteotomies.

In certain embodiments, the present disclosure describes methods for harvesting a bone graft for a surgical procedure. In one example, a patient-specific harvesting guide may be used to harvest an autograft bone graft from a bone of the patient. The autograft bone graft is sized by way of the patient-specific harvesting guide to match a dimension that satisfies a predefined bone graft dimension. In one embodiment, the dimension is the same as the predefined bone graft dimension. In another embodiment, the dimension is the substantially the same as the predefined bone graft dimension within an accepted tolerance level. The present disclosure describes apparatuses, systems, and/or methods for harvesting bone for a surgical procedure that address shortcomings of conventional solutions.

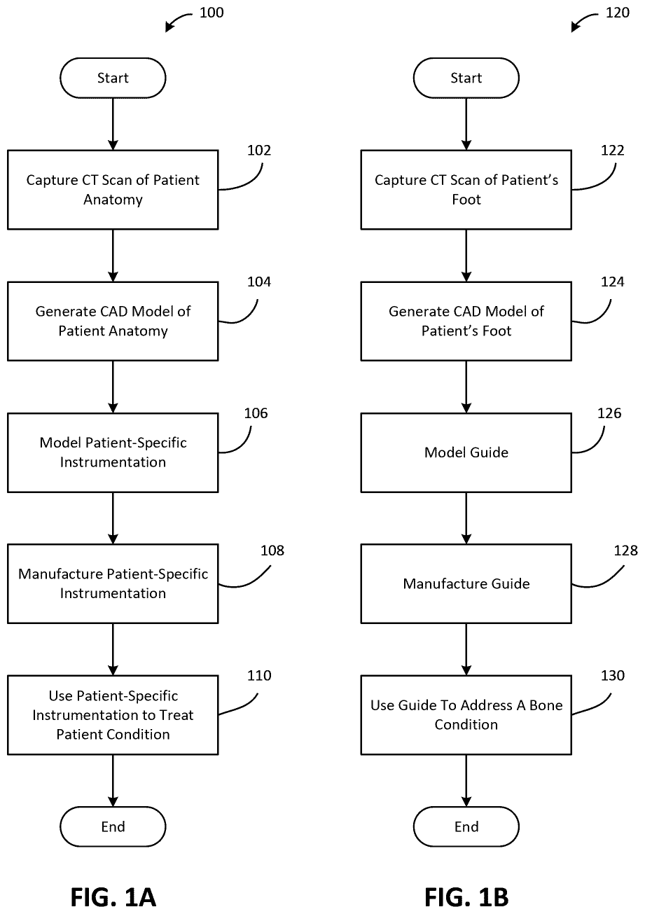

FIG. 1A is a flowchart diagram depicting a method 100 for correcting a bone condition, according to one embodiment. The method 100 may be used for any of a wide variety of bone conditions, including but not limited to deformities, fractures, joint failure, and/or the like. Further, the method 100 may provide correction with a wide variety of treatments, including but not limited to arthroplasty, arthrodesis, fracture repair, and/or the like.

As shown, the method 100 may begin with a step 102 in which a CT scan (or another three-dimensional image, also referred to as medical imaging) of the patient's anatomy is obtained. The step 102 may entail capturing a scan of only the particular bone(s) to be treated, or may entail capture of additional anatomic information, such as the surrounding tissues. Additionally, or alternatively, the step 102 may entail receiving a previously captured image, for example, at a design and/or fabrication facility. Performance of the step 102 may result in possession of a three-dimensional model of the patient's anatomy, or three-dimensional surface points that can be used to construct such a three-dimensional model.

After the step 102 has been carried out, the method 100 may proceed to a step 104 in which a CAD model of the patient's anatomy (including one or more bones) is generated. The CAD model may be one example of a bone model. The CAD model may be of any known format, including but not limited to SolidWorks, Catia, AutoCAD, or DXF. In some embodiments, customized software may be used to generate the CAD model from the CT scan. The CAD model may only include the bone(s) to be treated and/or may include surrounding tissues. In alternative embodiments, the step 104 may be omitted, as the CT scan may capture data that can directly be used in future steps without the need for conversion.

In one embodiment, the CAD model generated and/or patient-specific instrumentation, implants, and/or plan for conducting an operative procedure, may be enhanced by the use of advanced computer analysis system, machine learning, and/or automated/artificial intelligence. For example, these technologies may be used to revise a set of steps for a procedure such that a more desirable outcome is achieved.

In a step 106, the CAD model and/or CT scan data may be used to model patient-specific instrumentation that can be used to correct the condition, as it exists in the patient's anatomy. In some embodiments, any known CAD program may be used to view and/or manipulate the CAD model and/or CT scan and generate one or more instruments that are matched specifically to the size and/or shape of the patient's bone(s). In some embodiments, such instrumentation may include a targeting guide, trajectory guide, drill guide, cutting guide, tendon trajectory guide, positioner or positioning guide or similar guide that can be attached to one or more bones, with one or more features that facilitate work on the one or more bones pursuant to a procedure such as arthroplasty or arthrodesis. In some embodiments, performance of the step 106 may include modelling an instrument with a bone engagement surface that is shaped to match the contour of a surface of the bone, such that the bone engagement surface can lie directly on the corresponding contour.

In a step 108, the model(s) may be used to manufacture patient-specific instrumentation and/or implants. This may be done via any known manufacturing method, including casting, forging, milling, additive manufacturing, and/or the like. Additive manufacturing may provide unique benefits, as the model may be directly used to manufacture the instrumentation and/or implants (without the need to generate molds, tool paths, and/or the like beforehand). Such instrumentation may optionally include a targeting guide, trajectory guide, drill guide, cutting guide, positioning guide, positioner, a graft harvesting guide, or tendon trajectory guide with a bone engagement surface and/or one or more features as described herein.

In addition to, or in the alternative to the step 108, the model(s) may be used to select from available sizes of implants and/or instruments and advise the surgeon accordingly. For example, where a range of guides are available for a given procedure, analysis of the CAD data may facilitate pre-operative selection of the optimal guide and/or optimal placement of the guide on the bone. Similarly, if a range of implants may be used for a given procedure, analysis of the CAD data may facilitate pre-operative selection of the optimal implant(s). More particularly, properly-sized spacers, screws, bone plates, and/or other hardware may be pre-operatively selected.

Thus, the result of the step 108 may provision, to the surgeon, of one or more of the following: (1) one or more patient-specific instruments; (2) one or more patient-specific implants; (3) an instrument, selected from one or more available instrument sizes and/or configurations; (4) an implant, selected from one or more available implant sizes and/or configurations; (5) instructions for which instrument(s) to select from available instrument sizes and/or configurations; (6) instructions for which implant(s) to select from available implant sizes and/or configurations; (7) instructions for proper positioning or anchorage of one or more instruments to be used in the procedure; and (8) instructions for proper positioning or anchorage of one or more implants to be used in the procedure. These items may be provided to the surgeon directly, or to a medical device company or representative, for subsequent delivery to the surgeon.

In a step 110, the manufactured instrumentation may be used in surgery to facilitate treatment of the condition. In some embodiments, this may entail placing the modelled bone engagement surface against the corresponding contour of the bone used to obtain its shape, and then using the resection feature(s) to guide resection of one or more bones. Then the bone(s) may be further treated, for example, by attaching one or more joint replacement implants (in the case of joint arthroplasty), or by attaching bone segments together (in the case of arthrodesis or fracture repair). Prior to completion of the step 110, the instrumentation may be removed from the patient, and the surgical wound may be closed.

As mentioned previously, the method 100 may be used to correct a wide variety of bone conditions. One example of the method 100 will be shown and described in connection with FIG. 1B, for correction of a bunion deformity of the foot.

In certain embodiments, one or more of a method, apparatus, and/or system of the disclosed solution can be used for training a surgeon to perform a patient-specific procedure or technique. In one embodiment, the CAD model generated and/or patient-specific instrumentation, implants, and/or plan for conducting an operative procedure can be used to train a surgeon to perform a patient-specific procedure or technique.

In one example embodiment, a surgeon may submit a CT scan of a patient's foot to an apparatus or system that implements the disclosed solution. Next, a manual or automated process may be used to generate a CAD model and for making the measurements and correction desired for the patient. In the automated process, advanced computer analysis system, machine learning and automated/artificial intelligence may be used to generate a CAD model and/or one or more patient-specific instruments and/or operation plans. For example, a patient-specific guide may be fabricated that is registered to the patient's anatomy using a computer-aided machine (CAM) tool. In addition, a CAM tool may be used to fabricate a 3D structure representative of the patient's anatomy, referred to herein as a patient-specific synthetic cadaver. (e.g. one or more bones of a patient's foot). Next, the patient-specific guide and the patient-specific synthetic cadaver can be provided to a surgeon who can then rehearse an operation procedure in part or in full before going into an operating room with the patient.

In certain embodiments, the patient-specific guide or instrument can be used to preposition and/or facilitate pre-drilling holes for a plate system for fixation purposes. Such plate systems may be optimally placed, per a CT scan, after a correction procedure for optimal fixation outcome. In another embodiment, the CAD model and/or automated process such as advanced computer analysis, machine learning and automated/artificial intelligence may be used to measure a depth of the a through a patient-specific resection guide for use with robotics apparatus and/or systems which would control the depth of each cut within the guide to protect vital structures below or adjacent to a bone being cut. In another embodiment, the CAD model and/or automated process such as advanced computer analysis, machine learning and automated/artificial intelligence may be used to define desired fastener (e.g. bone screw) length and/or trajectories through a patient-specific guide and/or implant. The details for such lengths, trajectories, and components can be detailed in a report provided to the surgeon preparing to perform a procedure.

FIG. 1B is a flowchart diagram depicting a method 120 for correcting or remediating a bone condition, according to one embodiment. The method 120 may be used to prepare for an orthopedic procedure which corrects or remediates a bone, muscle, and/or tendon condition of a patient.

As shown, the method 120 may begin with a step 122 in which a CT scan (or another three-dimensional image) of the patient's foot is obtained. The step 122 may entail capturing a scan of only the first cuneiform and first metatarsal, or may entail capture of additional anatomic information, such as the entire foot. Additionally, or alternatively, the step 122 may entail receipt of previously captured image data. Capture of the entire foot in the step 122 may facilitate proper alignment of the first metatarsal with the rest of the foot (for example, with the second metatarsal). Performance of the step 122 may result in generation of a three-dimensional model of the patient's foot, or three-dimensional surface points that can be used to construct such a three-dimensional model.

After the step 122 has been carried out, the method 120 may proceed to a step 124 in which a CAD model of the relevant portion of the patient's anatomy is generated. The CAD model may optionally include the bones of the entire foot, like the CT scan obtained in the step 122. In alternative embodiments, the step 124 may be omitted in favor of direct utilization of the CT scan data, as described in connection with the step 104.

In a step 126, the CAD model and/or CT scan data may be used to model patient-specific instrumentation that can be used to correct or remediate a bone condition. Such instrumentation may include a guide. In one example, the guide can seat or abut or contact a surface of a bone and including an opening that guides a trajectory for a fastener for a procedure. In some embodiments, performance of the step 126 may include modelling the guide with a bone engagement surface that is shaped to match contours of the surfaces of the bone, such that the bone engagement surface can lie directly on the corresponding contours of the bone.

In a step 128, the model(s) may be used to manufacture patient-specific instrumentation and/or instruments. This may include manufacturing the guide with the bone engagement surface and/or other features as described above. As in the step 108, the step 128 may additionally or alternatively involve provision of one or more instruments and/or implants from among a plurality of predetermined configurations or sizes. Further, the step 128 may additionally, or alternatively, involve provision of instructions for placement and/or anchorage of one or more instruments and/or instruments to carry out the procedure.

In a step 130, the manufactured guide may be used in surgery to facilitate treatment of the condition. Specifically, the bone engagement surface of the guide may be placed against the corresponding contours of the bone. The guide may include an opening and/or trajectory guide to guide insertion of a trajectory guide such as a temporary fastener such as a K-wire. The guide may then be removed, and the remaining steps of a surgical procedure performed.

The method 100 and the method 120 are merely exemplary. Those of skill in the art will recognize that various steps of the method 100 and the method 120 may be reordered, omitted, and/or supplemented with additional steps not specifically shown or described herein.

As mentioned previously, the method 120 is one species of the method 100; the present disclosure encompasses many different procedures, performed with respect to many different bones and/or joints of the body. Exemplary steps and instrumentation for the method 120 will further be shown and described in connection with the present disclosure. Those of skill in the art will recognize that the method 120 may be used in connection with different instruments; likewise, the instruments of the present disclosure may be used in connection with methods different from the method 100 and the method 120.

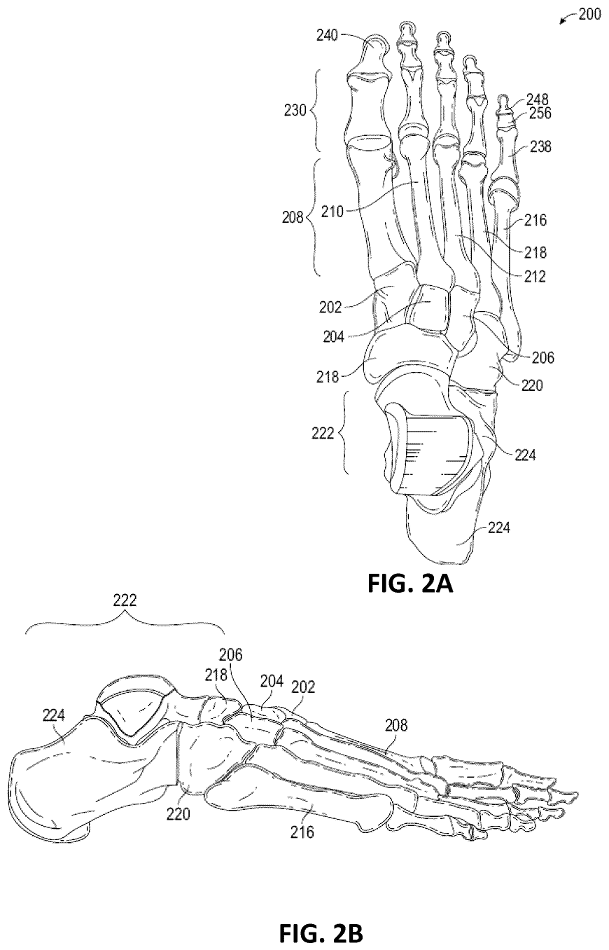

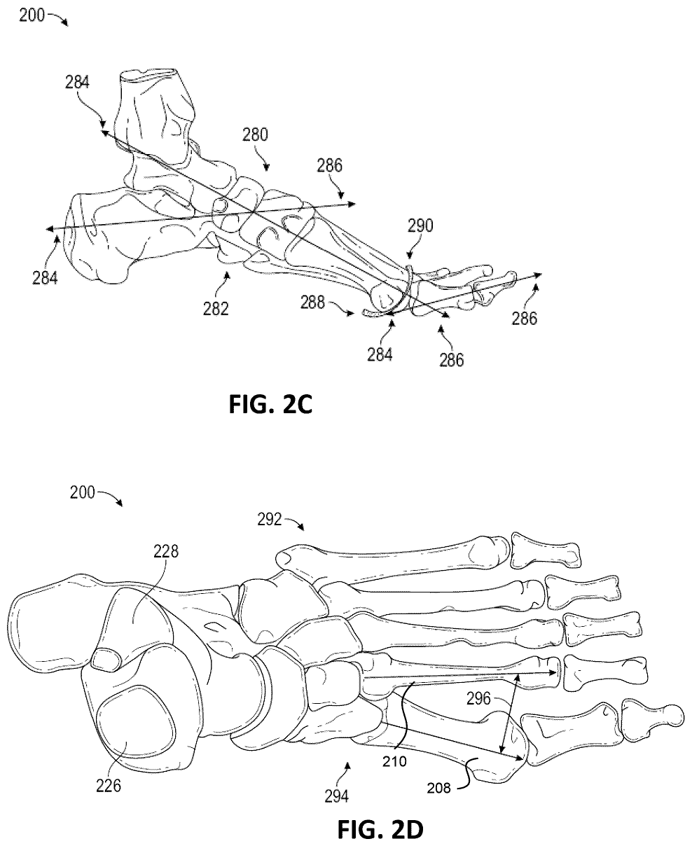

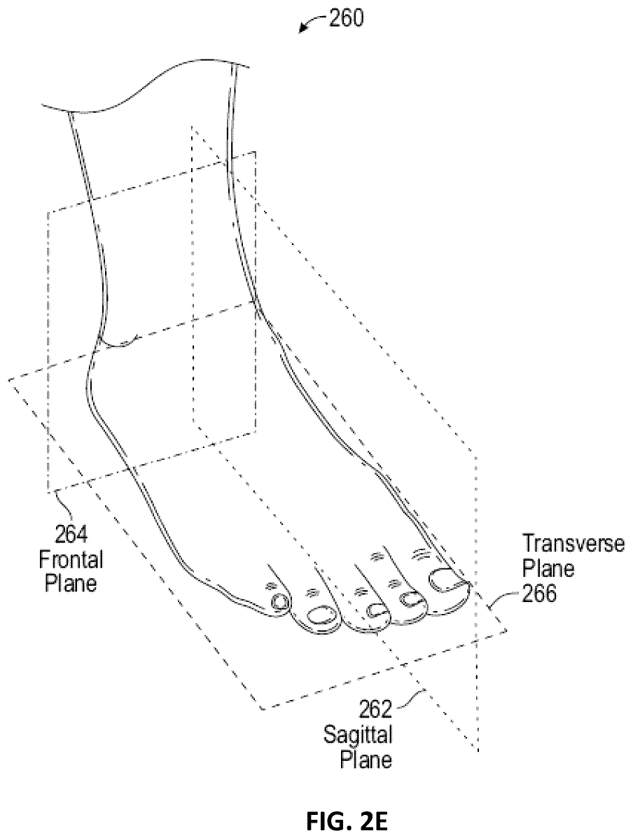

FIG. 2A is a perspective dorsal view of a foot 200. The foot 200 may have a medial cuneiform 202, an intermediate cuneiform 204, lateral cuneiform 206, a first metatarsal 208, a second metatarsal 210, third metatarsal 212, fourth metatarsal 214, fifth metatarsal 216, navicular 218, cuboid 220, and calcaneus 222, among others. The medial cuneiform 202 and the intermediate cuneiform 204 may be joined together at a first metatarsocuneiform joint, and the first metatarsal 208 and the second metatarsal 210 may be joined together at a second metatarsocuneiform joint. The foot 200 includes a set of proximal phalanges numbered first through fifth (230, 232, 234, 236, 238) and a set of distal phalanges numbered first through fifth (240, 242, 244, 246, 248) and a set of middle phalanges numbered second through fifth (250, 252, 254, 256).

FIG. 2B is a perspective lateral view of a foot 200, with bones of the foot labeled.

FIG. 2C is a perspective medial view of a foot illustrating a dorsal side 280 and a plantar side 282. Dorsal refers to the top of the foot. Plantar refers to the bottom of the foot. Proximal 284 is defined as “closer to the primary attachment point”. Distal 286 is defined as “further away from the attachment point”. Plantarflex or plantarflexion 288 means movement toward the plantar side 282 of a foot or hand, toward the sole or palm. Dorsiflex or dorsiflexion 290 means movement toward the dorsal side 280 of a foot or hand, toward the top. FIG. 2D is a perspective dorsal view of the foot 200. A transverse plane is the plane that shows the top of the foot. A lateral side 292 means a side furthest away from the midline of a body, or away from a plane of bilateral symmetry of the body. A medial side 294 means a side closest to the midline of a body, or toward a plane of bilateral symmetry of the body. For a Lapidus procedure, the intermetatarsal (IM) angle 296 is the angle to be corrected to remove the hallux valgus (bunion) deformity. The IM angle 296 is an angle between a longitudinal axis of a first metatarsal and a second metatarsal of a patient.

FIG. 2E is a view of a foot illustrating common planes 260 of reference for a human foot. FIG. 2E illustrates a sagittal plane 262 that divides the foot into a right section and a left section half. The sagittal plane 262 is perpendicular to frontal or coronal plane 264 and the transverse plane 266. In the foot, the frontal plane 264 generally runs vertically through the ankle and the transverse plane 266 generally runs horizontally through the midfoot and toes of the foot.

Every patient and/or condition is different; accordingly, the degree of angular adjustment needed in each direction may be different for every patient. Use of a patient-specific guide may help the surgeon obtain an optimal realignment, target, or position a bone tunnel, position one or more resections and/or fasteners and the like. Thus, providing patient-specific guides, jigs, and/or instrumentation may provide unique benefits.

The present patient-specific instrumentation may be used to correct a wide variety of bone conditions. Such conditions include, but are not limited to, any angular deformities from within one bone segment in either the lower or upper extremities (for example, tibial deformities, calcaneal deformities, femoral deformities, and radial deformities). The present disclosure may also be used to treat an interface between two bone segments (for example, the ankle joint, metatarsal cuneiform joint, 45avigate's joint, complex Charcot deformity, wrist joint, knee joint, etc.). As one example, an angular deformity or segmental malalignment in the forefoot may be treated, such as is found at the metatarsal cuneiform level, the midfoot level such as the navicular cuneiform junction, hindfoot at the calcaneal cuboid or subtalar joint or at the ankle between the tibia and talar junction. Additionally, patient-specific instruments could be used in the proximal leg between two bone segments or in the upper extremity such as found at the wrist or metacarpal levels.

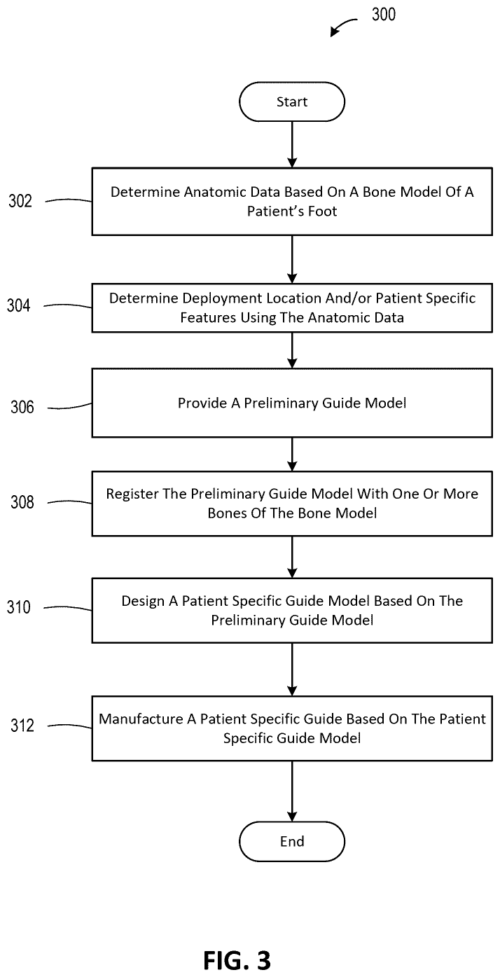

FIG. 3 illustrates a flowchart diagram depicting a method 300 for generating one or more patient-specific instruments configured to correct or address a bone or foot condition, according to one embodiment. Prior to steps of the method 300, a bone model (also referred to as CAD model above) is generated. The bone model may be generated using medical imaging of a patient's foot and may also be referred to as an anatomic model. The medical imaging image(s) may be used by computing devices to generate patient imaging data. The patient imaging data may be used to measure and account for orientation of one or more structures of a patient's anatomy. In certain embodiments, the patient imaging data may serve, or be a part of, anatomic data for a patient.

In one embodiment, the method 300 begins after a bone model of a patient's body or body part(s) is generated. In a first step 302, the method 300 may review the bone model and data associated with the bone model to determine anatomic data of a patient's foot.

After step 302, the method 300 may determine 304 a recommended location and/or a trajectory angle and/or patient-specific features for a procedure using the anatomic data. “Recommended location” refers to a location for deployment of guide or instrument on, in, between, or within one or more body parts (e.g., bones) of a patient. “Trajectory angle” refers to a recommended angle for deployment of an instrument, graft, body part, or resection feature angle relative to a bone of a patient for a procedure. In certain embodiments, determining the recommended location may employ advanced computer analysis system, expert systems, machine learning, and/or automated/artificial intelligence. In another embodiment, the method 300 may include determining one or more alternative locations and/or trajectory angles for instrumentation.

Next, the method 300 may proceed and a preliminary guide model is provided 306 from a repository of template instrumentation models. A preliminary guide model is a model of a preliminary guide.

As used herein, “preliminary guide” refers to a guide configured, designed, and/or engineered to serve as a template, prototype, archetype, or starting point for creating, generating, or fabricating a patient-specific guide. In one aspect, the preliminary guide may be used, as-is, without any further changes, modifications, or adjustments and thus become a patient-specific guide. In another aspect, the preliminary guide may be modified, adjusted, or configured to more specifically address the goals, objectives, or needs of a patient or a surgeon and by way of the modifications become a patient-specific guide. The patient-specific guide can be used by a user, such as a surgeon, to guide steps in a surgical procedure, such as an osteotomy. Accordingly, a preliminary guide model can be used to generate a patient-specific guide. The patient-specific guide model may be used in a surgical procedure to facilitate one or more steps of the procedure and may be used to generate a patient-specific guide that can be used in a surgical procedure for the patient.

In certain embodiments, the preliminary guide model may be generated based on anatomic data and/or a bone model or a combination of these, and no model or predesigned structure, template, or prototype. Alternatively, or in addition, the preliminary guide model may be, or may originate from, a template guide model selected from a set of template guide models. Each model in the set of template guide models may be configured to fit an average patient's foot. The template guide model may subsequently be modified or revised by an automated process or manual process to generate the preliminary guide model used in this disclosure.

As used herein, “template guide” refers to a guide configured, designed, and/or engineered to serve as a template for creating, generating, or fabricating a patient-specific guide. In one aspect, the template guide may be used, as-is, without any further changes, modifications, or adjustments and thus become a patient-specific guide. In another aspect, the template guide may be modified, adjusted, or configured to more specifically address the goals, objectives, or needs of a patient or a surgeon and by way of the modifications become a patient-specific guide. The patient-specific guide can be used by a user, such as a surgeon, to guide making one or more resections of a structure, such as a bone for a procedure. Accordingly, a template guide model can be used to generate a patient-specific guide model. The patient-specific guide model may be used in a surgical procedure to address, correct, or mitigate effects of the identified deformity and may be used to generate a patient-specific guide that can be used in a surgical procedure for the patient.

Next, the method 300 may register 308 the preliminary guide model with one or more bones of the bone model. This step 308 facilitates customization and modification of the preliminary guide model to generate a patient-specific guide model from which a patient-specific guide can be generated. The registration step 308 may combine two models and/or patient imaging data and positions both models for use in one system and/or in one model.

Next, the method 300 may design 310 a patient-specific guide model based on the preliminary guide model. The design step 310 may be completely automated or may optionally permit a user to make changes to a preliminary guide model or partially completed patient-specific guide model before the patient-specific guide model is complete. A preliminary guide model and patient-specific guide model are two examples of an instrument model. As used herein, “instrument model” refers to a model, either physical or digital, that represents an instrument, tool, apparatus, or device. Examples of an instrument model can include a cutting guide model, a resection guide model, an alignment guide model, a reduction guide model, a patient-specific tendon trajectory guide model, a positioner model, a positioning guide model, and the like. In one embodiment, a patient-specific guide and a patient-specific guide model may be unique to a particular patient and that patient's anatomy and/or condition.

The method 300 may conclude by a step 312 in which patient-specific guide may be manufactured based on the patient-specific guide model. Various manufacturing tools, devices, systems, and/or techniques can be used to manufacture the patient-specific guide.

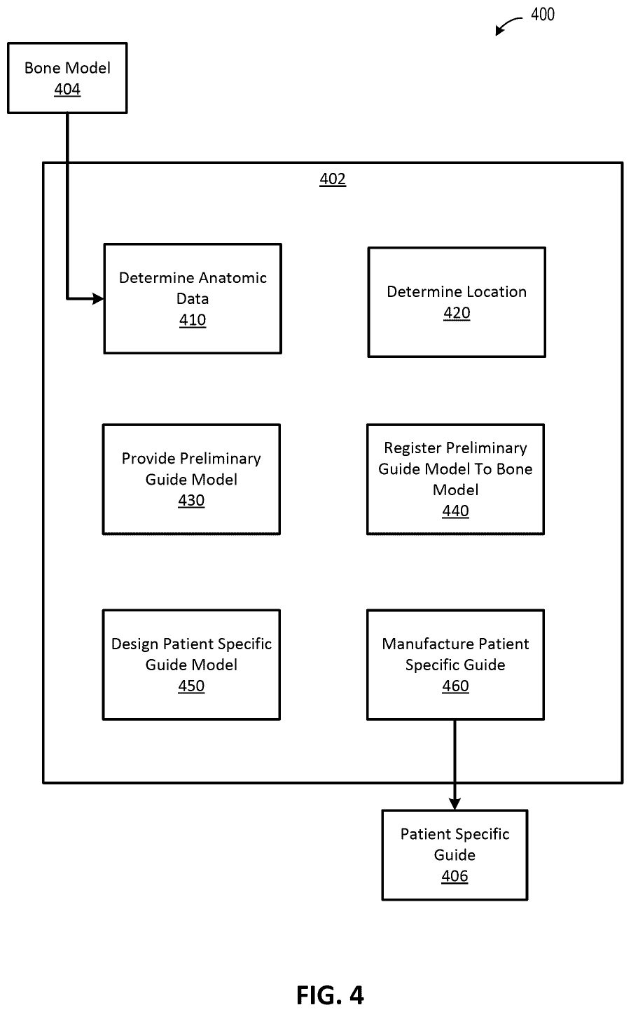

FIG. 4 illustrates an exemplary system 400 configured to generate one or more patient-specific instruments configured to facilitate surgical procedures, according to one embodiment. The system 400 may include an apparatus 402 configured to accept, review, receive or reference a bone model 404 and provide a patient-specific guide 406. In one embodiment, the apparatus 402 is a computing device. In another embodiment, the apparatus 402 may be a combination of computing devices and/or software components or a single software component such as a software application.

The apparatus 402 may include a determination module 410, a location module 420, a provision module 430, a registration module 440, a design module 450, and a manufacturing module 460. Each of which may be implemented in one or more of software, hardware, or a combination of hardware and software.

The determination module 410 determines anatomic data 412 from a bone model 404. In certain embodiments, the system 400 may not include a determination module 410 if the anatomic data is available directly from the bone model 404. In certain embodiments, the anatomic data for a bone model 404 may include data that identifies each anatomic structure within the bone model 404 and attributes about the anatomic structure. For example, the anatomic data may include measurements of the length, width, height, and density of each bone in the bone model. Furthermore, the anatomic data may include position information that identifies where each structure, such as a bone is in the bone model 404 relative to other structures, including bones. The anatomic data may be in any suitable format and may be stored separately or together with data that defines the bone model 404.

In one embodiment, the determination module 410 may use advanced computer analysis system such as image segmentation to determine the anatomic data. The determination module 410 may determine anatomic data from one or more sources of medical imaging data, images, files, or the like. Alternatively, or in addition the determination module 410 may use software and/or systems that implement one or more artificial intelligence methods (e.g., machine learning and/or neural networks) for deriving, determining, or extrapolating, anatomic data from medical imaging or the bone model. In one embodiment, the determination module 410 may perform an anatomic mapping of the bone model 404 to determine each unique aspect of the intended osteotomy procedure and/or bone resection and/or bone translation. The anatomic mapping may be used to determine coordinates to be used for an osteotomy procedure, position and manner of resections to be performed either manually or automatically or using robotic surgical assistance, a depth/length/span/thickness for bone cuts, an angle for bone cuts, a predetermined depth for bone cuts, dimensions and configurations for resection instruments such as saw blades, milling bit size and/or speed, saw blade depth markers, and/or instructions for automatic or robotic resection operations.

In one embodiment, the determination module 410 may use advanced computer analysis system such as image segmentation to determine the anatomic data. The determination module 410 may determine anatomic data from one or more sources of medical imaging data, images, files, or the like. The determination module 410 may perform the image segmentation using 3D modeling systems and/or artificial intelligence (AI) segmentation tools. In certain embodiments, the determination module 410 is configured to identify and classify portions of bone based on a condition of the bone, based on the bone condition. Such classifications may include identifying bone stability, bone density, bone structure, bone deformity, bone structure, bone structure integrity, and the like. Accordingly, the determination module 410 may identify portions or sections or one or more bones based on a quality metric for the bone. Advantageously, that determination module 410 can identify high quality bone having a viable structure, integrity, and/or density versus lower quality bone having a nonviable structure, integrity, and/or density and a plurality of bone quality levels in between.

Accordingly, the determination module 410 can guide a surgeon to determine which areas of one or more bones of a patient are within a “soft tissue envelope” (bone of undesirable quality) as that bone relates to a particular deformity or pathology. Identifying the quality of one or more bones of the patient can aid a surgeon in determining what type of correction or adjustment is needed. For example, an ulceration that occurs due to a boney deformity can be mapped using the determination module 410 in a way that a correction can be performed to correct the deformity and reduce pressure to an area and address the structures that were causing the pressure ulceration/skin breakdown.

In addition, the determination module 410 and/or another component of the apparatus 402 can be used to perform anatomic mapping which may include advanced medical imaging, such as the use of CT scan, ultrasound, MRI, and bone density scans can be combined to effectively create an anatomic map that determines the structural integrity of the underlying bone.

Identifying the structural integrity of the underlying bone can help in determining where bone resections can be performed to preserve the densest bone in relation to conditions such as Charcot neuropathic, arthropathy where lesser dense bone can fail and collapse. It is well documented in the literature that failure to address and remove such lesser dense bone can ultimately lead to failure of a reconstruction and associated hardware.

The present disclosure provides, by way of at least the exemplary system 400, an anatomic map that can be part of anatomic data. The anatomic map can combine structural, deformity, and bone density information and can be utilized to determine the effective density of bone and help to determine where bone should be resected in order to remove the lesser dense bone while maintaining more viable bone to aid in the planning of the osteotomy/bone resection placement.

The location module 420 determines or identifies one or more recommended locations and/or trajectory angles for deployment of an instrument, graft, and/or soft tissue based on the anatomic data 412 and/or the bone model 404. In one embodiment, the location module 420 may compare the anatomic data 412 to a general model that is representative of most patient's anatomies and may be free from deformities or anomalies. The location module 420 can operate autonomously and/or may facilitate input and/or revisions from a user. The location module 420 may be completely automated, partially automated, or completely manual. A user may control how automated or manual the determining of the location and/or trajectory angles is.

The provision module 430 is configured to provide a preliminary guide model 438. The provision module 430 may use a variety of methods to provide the preliminary guide model. In one embodiment, the provision module 430 may generate a preliminary guide model. In the same, or an alternative embodiment, the provision module 430 may select a template guide model for a surgical procedure configured to enable locating the position for one or more instruments and/or providing a trajectory provided by the location module 420. In one embodiment, the provision module 430 may select a template guide model from a set of template guide models (e.g., a library, set, or repository of template guide models).

The registration module 440 registers the preliminary guide model with one or more bones or other anatomical structures of the bone model 404. As explained above, registration is a process of combining medical imaging data, patient imaging data, and/or one or more models such that the preliminary guide model can be used with the bone model 404.

The design module 450 designs a patient-specific guide (or patient-specific guide model) based on the preliminary guide model. The design operation of the design module 450 may be completely automated, partially automated, or completely manual. A user may control how automated or manual the designing of the patient-specific guide (or patient-specific guide model) is.

The manufacturing module 460 may manufacture a patient-specific guide 406 using the preliminary guide model. The manufacturing module 460 may use a patient-specific guide model generated from the preliminary guide model. The manufacturing module 460 may provide the patient-specific guide model to one or more manufacturing tools and/or fabrication tool. The patient-specific guide model may be sent to the tools in any format such as an STL file or any other CAD modeling or CAM file or method for data exchange. In one embodiment, a user can adjust default parameters for the patient-specific guide such as types and/or thicknesses of materials, dimensions, and the like before the manufacturing module 460 provides the patient-specific guide model to a manufacturing tool.

Effective connection of the guide to one or more bones can ensure that surgical steps are performed in desired locations and/or with desired orientations and mitigate undesired surgical outcomes.

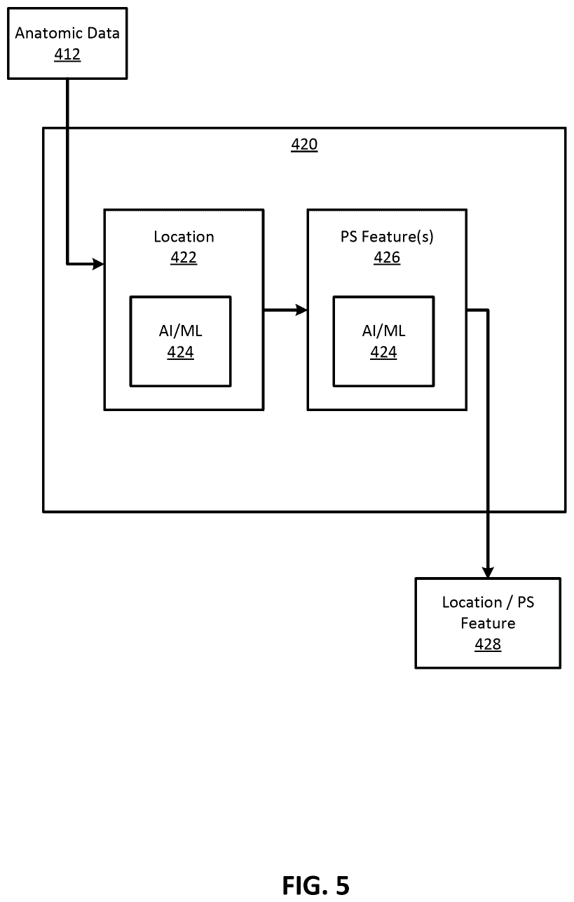

FIG. 5 illustrates an exemplary location module 420 configured to determine a recommended location and/or trajectory for steps and/or instruments in a surgical procedure, according to one embodiment. The location module 420 may factor in one or more landmarks on one or more surfaces of one or more bones of a patient of the bone model 404. The location module 420 may be completely automated, partially automated, or completely manual. A user may control how automated or manual the determination of the recommended location is. The user may provide instructions to the location module 420 to facilitate automatic or partially automated determination of one or more recommended locations.

The location module 420 may include a location module 422. The location module 422 may be configured for automated determination of a recommended location for steps and/or instruments in a surgical procedure. For example, in one embodiment, the location module 422 includes an artificial intelligence or machine learning module 424. The artificial intelligence or machine learning module 424 is configured to implement one or more of a variety of artificial intelligence modules that may be trained for identifying bones in the bone model 404, determining surfaces and/or sides of one or more bones, determining landmarks (both natural and/or abnormalities), determining axes of a bone, such as a longitudinal axis and/or a horizontal axis of a bone based on anatomic data 412 and/or a bone model 404. In another embodiment, the location module 420 may receive patient imaging data, a bone model, a CAD model or the like and use these inputs to determine a recommended location and/or trajectory in relation to one or more bones of a patient.

In one embodiment, the artificial intelligence or machine learning module 424 may be trained using a large data set of anatomic data 412 for healthy bones and a large data set of anatomic data 412 for bones with abnormalities and/or landmarks in which the abnormalities and/or landmarks have been previously identified and labeled in the dataset. The artificial intelligence or machine learning module 424 may implement, or use, a neural network configured according to the training such that as the artificial Intelligence or machine learning module 424 accepts the anatomic data 412 for a particular patient, the artificial intelligence or machine learning module 424 is able to determine what one or more locations (e.g., a recommended location and one or more alternative locations for the guide.

The location module 422 may interact with a patient specific feature module 426. The patient specific feature module 426 may take one or more locations provided by the location module 422 and the bone model 404 and/or anatomic data 412 and determine suitable patient specific features. In certain embodiments, the patient specific features provided by the patient specific feature module 426 may include a number of resection features, an angle or trajectory for one or more resection features, a number, size, and/or position of bone attachment features, a number, size, or position of alignment guides or a combination of these. In certain embodiments, the patient specific feature module 426 may focus on resection features.

As with the location module 420, the patient specific feature module 426 may be completely automated, partially automated, or completely manual. A user may control how automated or manual the determination of the trajectory is. The user may provide instructions to the patient specific feature module 426 to facilitate automatic or partially automated determination of one or more trajectories. In one embodiment, the location module 422 includes an artificial intelligence or machine learning module 424 that facilitates determining one or more trajectories.

The location module 420 outputs a location/patient specific feature 428 for an orthopedic surgical procedure.

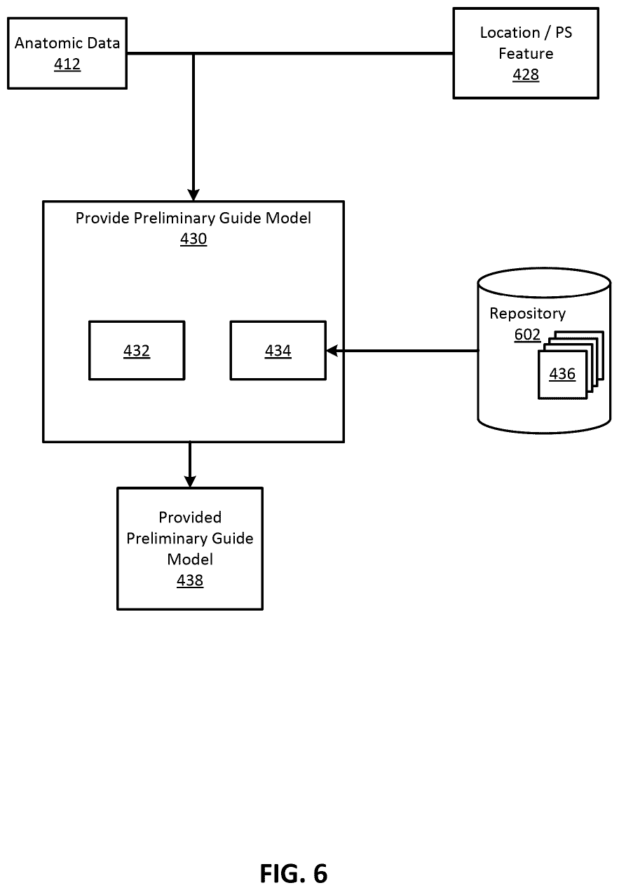

FIG. 6 illustrates an exemplary provision module 430 configured to provide a preliminary guide model, according to one embodiment. The provision module 430 may accept anatomic data 412 and a location/patient specific feature 428. In the illustrated embodiment, the provision module 430 may generate a preliminary guide model 438 (e.g., generate from ‘scratch’) or the provision module 430 may select a template guide model 436 automatically from a set of template guide models 436 stored in a repository 602. The provision module 430 may incorporate a variety of parameters in order to provision, generate, determine, or select a template guide model 436. For example, in addition to the anatomic data 412, the provision module 430 may include patient imaging data, deformity parameters for a variety of angular deformities (in all 3 planes) of the midfoot or hind foot and ankle, patient preferences, and/or surgeon input parameters.

In one embodiment, the provision module 430 may include a generator 432 and/or a selection module 434. In one embodiment, the generator 432 is configured to generate a preliminary guide model 438. In certain embodiments, the generator 432 may generate or create the preliminary guide model based on anatomic data and/or a bone model or a combination of these and no other inputs. (e.g. no model or predesigned structure, template, or prototype). Alternatively, or in addition, the generator 432 may generate or create the preliminary guide model using a standard set of features or components that can be combined to form the preliminary guide model. The generated preliminary guide model may subsequently be modified or revised by an automated process, and/or manual process, to generate the preliminary guide model used in this disclosure.

The selection module 434 may be configured to select a template guide model 436 for an osteotomy procedure configured to correct the deformity identified by the location module 420. In one embodiment, the provision module 430 may select a template guide model 436 from a set of template guide models 436 (e.g., a library, set, or repository of template guide models 436). In one embodiment, the template guide model 436 may include digital models. In another embodiment, the template guide model 436 may include physical models. In such an embodiment, the repository 602 may be a warehouse or other inventory repository. Where the template guide model 436 are physical models, the systems, modules, and methods of this disclosure can be used and the physical model may be milled or machined (e.g., a CNC machine) to form a patient-specific guide that conforms to the bone surfaces of the patient.

Selection of a suitable template guide model 436 may be completely automated and/or may be partially automated and/or may depend on confirmation from a user before a generated preliminary guide model or a proposed template guide model 436 becomes the preliminary guide model 438. In another embodiment, the selection module 434 may facilitate a manual selection by a user of a template guide model 436 that would become the preliminary guide model 438. The selection module 434 may use the anatomic data 412 or the bone model 404 or a combination of these to select a suitable template guide model that would become the preliminary guide model 438.

In another embodiment, the generator 432 may facilitate revisions or edits by a user of a generated guide model that will become the preliminary guide model 438. The selection module 434 may use the anatomic data 412 or the bone model 404 or a combination of these to select a suitable template guide model that would become the preliminary guide model 438.

The repository 602 may include any number of, and/or a variety of template guide models 436. The template guide models 436 may be distinguished based on a gender or age of the patient, which joint of a midfoot, hind foot, or ankle will be cut, which material will be used for the template guide, and the like. The template guide model 436 may differ from each other in what degree of deformity correction the template guide model 436 is designed to provide. In addition, the template guide models 436 may be distinguished based on how one or more features of the template guide model 436 are positioned, arranged, and/or configured relative to each other. For example, in certain template guide models 436, the number, position, and/or configuration of alignment features and/or bone attachment features (e.g., holes) may vary based on needs or preferences of patients, the nature of the deformity, and/or surgeon preferences.

In certain embodiments, the template guide models 436 may vary in how the slots (e.g., resection features) for the cuts are positioned, angled, and oriented relative to each other and/or to a longitudinal axis of respective bones at a joint for use with the template guide model 436. For example, in one template guide model 436 the slot 1352 for a resection of a metatarsal bone may be perpendicular to a longitudinal axis of the metatarsal bone and the slot 1350 may be angled relative to a longitudinal axis of the resection or cuboid bone such that once the two bones are brought together the deformity is corrected. Alternatively, in another template guide model 436 the slot for a resection of a metatarsal bone may be angled relative to a longitudinal axis of the metatarsal bone and the slot 1350 may be perpendicular to a longitudinal axis of the cuneiform or cuboid bone such that once the two bones are brought together the deformity is corrected.

The selection module 434 may be configured to automatically select a template guide model 436 and/or provide an automatic template guide model 436 recommendation that can be changed by a user, such as a surgeon. For example, in one embodiment, the provision module 430 and/or selection module 434 includes an artificial intelligence or machine learning module. The artificial intelligence or machine learning module is configured to implement one or more of a variety of artificial intelligence modules that may be trained for selecting a template guide model 436 based on anatomic data 412 and/or other input parameters. In one embodiment, the artificial intelligence or machine learning module may be trained using a large data set of anatomic data 412 for suitable template guide models 436 identified and labeled in the dataset by professionals for use to treat a particular deformity. The artificial intelligence or machine learning module may implement, or use, a neural network configured according to the training such that the artificial intelligence or machine learning module is able to select a suitable template guide model 436. The template guide model 436 selected by the selection module 434 can become the preliminary guide model 438.

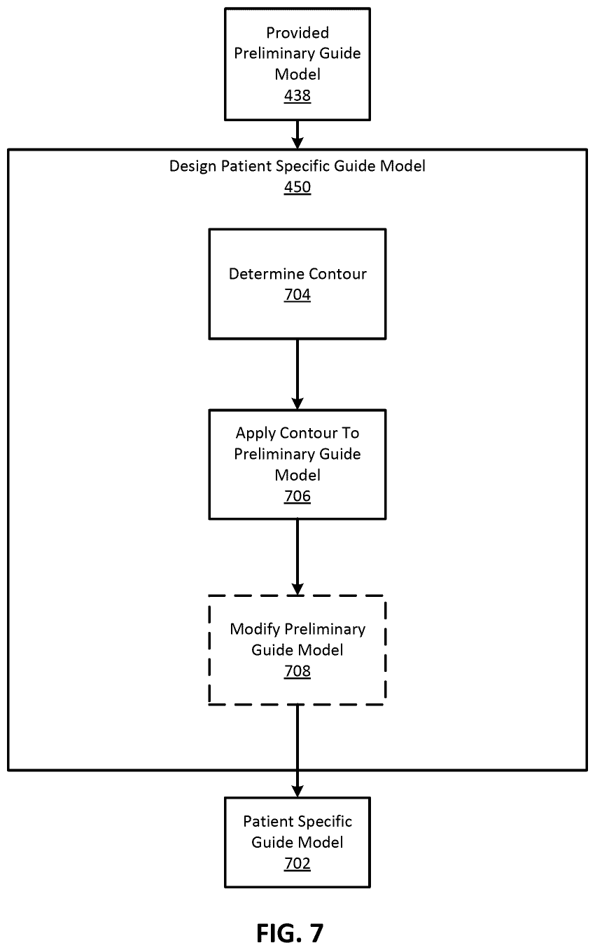

FIG. 7 illustrates an exemplary design module 450 configured to design a patient-specific guide model, according to one embodiment. The design module 450 may accept a preliminary guide model 438 and generate a patient-specific guide model 702. In one embodiment, the design module 450 includes a contour module 704, an application module 706, and/or an optional modification module 708.

Referring now to FIG. 7, the design module 450 may modify the preliminary guide model 438 such that the bone-facing and/or bone-contacting surfaces of the preliminary guide model 438 match a contour of the surfaces and/or joint of one or more bones where a step of an orthopedic procedure will be performed using the preliminary guide model 438.

The contour module 704 may determine a contour of the bones that will contact the preliminary guide model 438. The contour module 704 may use a bone model 404 and/or anatomic data 412 to determine the contour. For example, the contour module 704 may determine the shape of a dorsal surface of a calcaneus 222.

The application module 706 may apply the contour to the provided preliminary guide model 438 to custom contour a bone engagement surface of the preliminary guide model 438 to match the shape, contour, and/or one or more landmarks of a bone, such as a dorsal surface of a calcaneus 222. Applying the contour to the preliminary guide model 438 may convert the preliminary guide model 438 to a patient-specific guide model 702.

Generation of the contours of bone engagement surface of the preliminary guide model 438 may be performed in various CAD programs. In some embodiments, the shapes of the corresponding surface dorsal surface of a calcaneus 222 may be obtained directly from the bone model 404, anatomic data 412, CAD models and/or CT scan data, and simply copied onto the preliminary guide model 438. Various operations may be used to copy surfaces from one object to another. Additionally or alternatively, various Boolean operations, such as a Boolean subtraction operation, may be used to remove material from a model for the body of the preliminary guide model 438 with a shape that matches the dorsal surface of a calcaneus 222.

In certain embodiments, the design module 450 may include an optional module, such as a modification module 708. The modification module 708 may enable a user such as a technician or surgeon to make additional modifications to the design and configuration of the preliminary guide model 438. In one embodiment, the user can change any of the features, trajectories, fixation holes, handle engagement holes, angles, configurations, or parameters of the preliminary guide model 438. For example, a surgeon may be aware of other concerns or anatomic aspects of a patient, for example on an opposite foot or in connection with a hip or other orthopedic joint which motivate the surgeon to adjust an angle of one of more trajectories of the preliminary guide model 438.

Alternatively, or in addition, a user may use the modification module 708 to modify a predefined graft harvesting procedure. The user may add, remove, or modify steps and/or the instrumentation used in the graft harvesting procedure to create a patient-specific or patient tailored graft harvesting procedure and/or patient-specific graft harvesting instrument. In this manner, a user may configure features of a preliminary guide model 438 or modified preliminary guide model and/or graft harvesting procedure specific to a patient-specific osteotomy procedure the surgeon is planning for the patient.

The user may review the preliminary guide model 438 and make adjustments or revisions or make no adjustments or revisions. The output of the modification module 708 and/or the application module 706 is a patient-specific guide model 702.

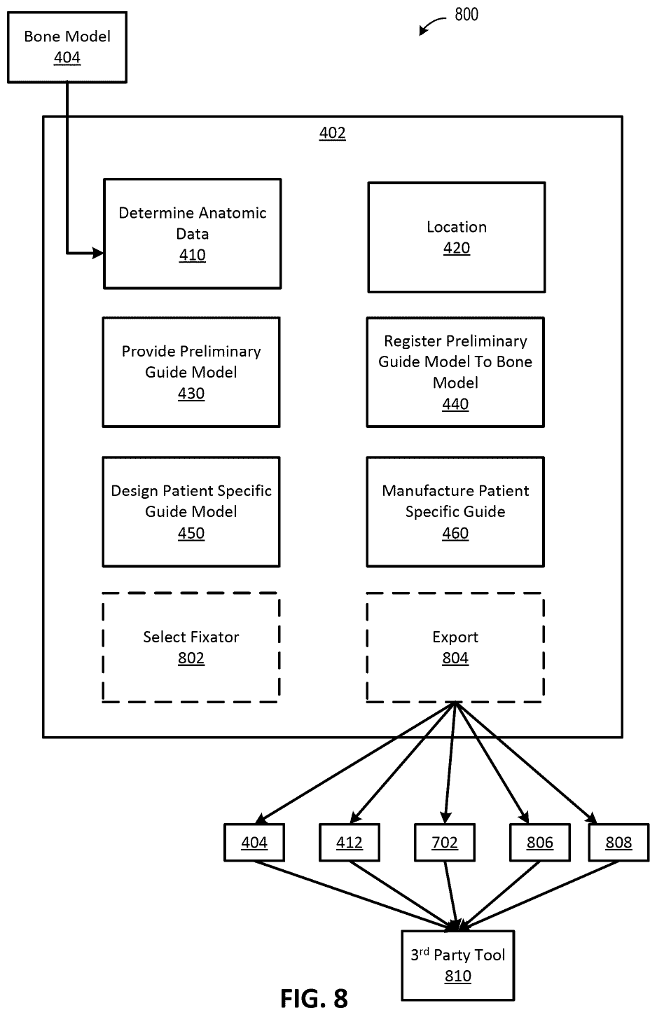

FIG. 8 illustrates an exemplary system 800 configured to generate one or more patient-specific instruments configured to correct a bone condition, according to one embodiment. The system 800 may include similar components or modules to those described in relation to FIG. 4. In addition, the system 800 may include a fixator selector 802 and/or an export module 804.

The fixator selector 802 enables a user to determine which fixator(s) to use for a graft harvesting procedure planned for a patient. In one embodiment, the fixator selector 802 may recommend one or more fixators based on the bone model 404, the location, the trajectory, or input from a user or a history of prior graft harvesting procedures performed. The fixator selector 802 may select a fixator model from a set of predefined fixator models or select a physical fixator from a set of fixators. The fixators may include a plate and associated accessories such as screws, anchors, and the like.

In one embodiment, the fixator selector 802 includes an artificial intelligence or machine learning module. The artificial intelligence or machine learning module is configured to implement one or more of a variety of artificial intelligence modules that may be trained for selecting fixator(s) based on anatomic data 412 and/or other input parameters. In one embodiment, the artificial intelligence or machine learning module may be trained using a large data set of anatomic data 412 for suitable fixator(s) identified and labeled in the dataset by professionals for use to treat a particular condition. The artificial intelligence or machine learning module may implement, or use, a neural network configured according to the training such that the artificial intelligence or machine learning module is able to select or recommend suitable fixator(s).

The export module 804 is configured to enable exporting of a patient-specific guide model 702 for a variety of purposes including, but not limited to, fabrication/manufacture of a patient-specific guide 406 and/or fixator(s), generation of a preoperative plan, generation of a physical bone model matching the bone model 404, and the like. In one embodiment, the export module 804 is configured to export the bone model 404, anatomic data 412, a patient-specific guide model 702, a preoperative plan 806, a fixator model 808, or the like. In this manner the custom instrumentation and/or procedural steps for a procedure (e.g., a graft harvesting procedure) can be used in other tools. The preoperative plan 806 may include a set of step by step instructions or recommendation for a surgeon or other staff in performing a procedure (e.g., a graft harvesting procedure). The preoperative plan 806 may include images and text instructions and may include identification of instrumentation to be used for different steps of the procedure (e.g., a graft harvesting procedure). The instrumentation may include the patient-specific guide 406 and/or one or more fixators/fasteners. In one embodiment, the export module 804 may provide a fixator model which can be used to fabricate a fixator for the procedure.

The exports (404, 412, 702, 806, and 808) may be inputs for a variety of 3^{rd} party tools 810 including a manufacturing tool, a simulation tool, a virtual reality tool, an augmented reality tool, an operative procedure simulation tool, a robotic assistance tool, and the like. A surgeon can then use these tools when performing a procedure or for rehearsals and preparation for the procedure. For example, a physical model of the bones, patient-specific guide 406, and/or fixators can be fabricated, and these can be used for a rehearsal operative procedure. Alternatively, a surgeon can use the bone model 404, preliminary guide model 438, and/or a fixator model to perform a simulated procedure using an operative procedure simulation tool.

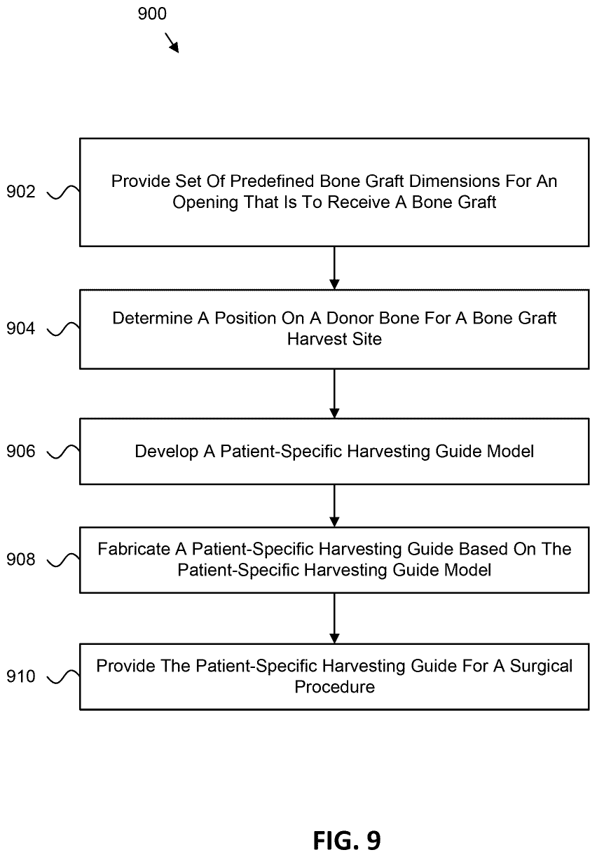

FIG. 9 is a flowchart of an example method 900. In some implementations, one or more method steps of FIG. 9 may be performed by a system 400, system 800, and/or a user. The present disclosure leverages the use of models, such as computer models, and particularly models of a specific patient to provide and/or generate instrumentation, implants, and/or surgical plans that advance patient care. Advantageously, these models are unique and customized for a particular patient. Thus, the models reflect the actual anatomical features and aspects of the patient.

By using models specific to a particular patient, a preoperative surgical plan can be prepared, engineered, and/or designed for a planned surgical procedure. The preoperative surgical plan may include one or more surgical osteotomies. Those of skill in the art will appreciate a variety of surgical osteotomies can be performed including an opening wedge osteotomy, a complete resection, a deformity correction, a correction for a nonunion, a malunion, or the like. In certain embodiments, the surgical procedure includes deploying a graft to a graft site that includes one or more surgical osteotomies. The graft can be an autograft, allograft, or xenograft. The graft can be of a variety of shapes, sizes, and other dimensions. For example, the graft can be an open wedge osteotomy. The purpose for the graft can be manifold or single fold and includes goals such as filling an opening caused by removal of deformed, fractures, injured, weak, or otherwise undesirable sections of one or more bones in, on, or around one or more joints, changing an orientation of one or more bones, lengthening or shortening one or more bones, fusing one or more joints, and the like.

One of the challenges with surgical osteotomies or surgical procedures, that include deployment of a graft, is determining the size and/or dimensions and/or configuration for the graft that will be deployed. Preferably, the graft is harvested from bone (referred to herein as “donor bone”) of the patient. Conventional approaches to overcoming this challenge include harvesting an autograft that is larger than the planned graft and then shaping or resizing this larger autograft on a back table of the operating room during the surgical procedure. One problem with the conventional approach is that the surgeon must estimate how much bone tissue to remove and on which sides of the autograft. If a surgeon removes too much, an autograft can be ruined, and a new autograft must be procured. Consequently, to mitigate such a risk, a surgeon may opt to remove very small amounts from the autograft and repeatedly check, and recheck, the size of the autograft with the size of the opening that will receive the autograft until sufficient bone tissue has been removed and or the size of the autograft is such that it is suitable for deployment in the opening. This trial-and-error approach can add time to the surgical procedure and increase stress on the surgeon who performs the surgical procedure.

Another challenge is determining how and where to resect a bone graft from a donor bone. Often a calcaneus is a desirable donor bone for a bone graft. However, a calcaneus may have limited landmarks and may present a smooth surface that can require a high degree of accuracy and expertise for a surgeon to secure a bone graft of the desired size and shape. Advantageously, the present disclosure provides a harvesting guide that facilitates resecting the bone graft from a bone graft harvest site, including but not limited to a calcaneus of a patient.

Advantageously, because the present disclosure teaches embodiments that are based on models of a patient's anatomy and/or preoperative planning that seeks to plan the surgical procedure from start to finish before the surgical procedure, a surgeon can determine one or more dimensions of an opening that is to receive an autograft during preoperative planning. In fact, a surgeon, user, computing device, automated system, and/or semiautomated system, during the preoperative planning stages can determine a shape for the autograft as well as a size. For example, an opening that is formed by a surgical osteotomy can be measured to determine exact or approximate depth/length/span/thickness, width, and height of the opening. The ability to preoperative plan the opening size and configuration enables a surgeon to also preoperative plan and prepare for bone graft harvesting, in particular autograft bone graft harvesting.

A model of an autograft can be built using modeling software such that the autograft has the same or approximately the same dimensions as the opening. Alternatively, or in addition, a surgeon or technician can have the autograft bone graft model designed such that one or more of the dimensions of the autograft are larger or smaller than the dimensions of the opening to provide an autograft that will accomplish the desired outcome of the surgical procedure. For example, to ensure adequate compression during healing a harvested autograft bone graft may be slightly larger than a depth/length/span/thickness of an opening such that fixation of the bone graft in the opening provides sufficient compression to encourage new bone growth. The larger size may be useful for example where the bone graft is a wedge-shaped bone graft positioned between two bone sections connected by a living hinge.

Using preoperative planning software, which may include modeling software, a bone graft model can be designed. Based on the bone graft model a harvesting guide can be designed that will assist a surgeon in harvesting an autograft bone graft having the same, or substantially the same dimensions as the bone graft model. In this manner, the dimensions of the autograft bone graft that is harvested are predetermined and/or predefined. In one embodiment, the dimensions are the same as the opening defined within a model of bones representing one or more stages of a planned surgical procedure for a patient. Advantageously, the model bones are substantially the same in all relevant aspects to the bones of the patient. The dimensions for an opening that is to be filled by an autograft bone graft and/or the dimensions of a harvested autograft bone graft are referred to herein as predefined bone graft dimensions.

In the present disclosure, embodiments are described with respect to surgical osteotomies on one or more bones of the foot and or ankle. However, those of skill in the art will appreciate that the apparatuses, systems, and methods can readily be used and or applied to surgical osteotomies including harvesting of autograft from bones other than those of the foot and ankle and such embodiments are included within the scope of this disclosure.

Just by way of example, a surgical osteotomy could be performed on the patient's hands, wrist, shoulder, or knee as well as the ankle or foot. Similarly, the bone (aka “bone graft harvest site”) used for harvesting an autograft can be any bone of the foot such as the calcaneus, any bone of the ankle such as distal part of the tibia a distal part of the fibula, a proximal part of the tibia, a proximal part of the tibia, or the like. The bone graft harvest site can also be on bones of the hip.

As shown in FIG. 9, method 900 may include providing a set of predefined bone graft dimensions for an opening that is to receive a bone graft, the set of predefined bone graft dimensions can be unique to a patient (block 902). Specifically, the set of predefined bone graft dimensions can include a length/span/thickness and/or depth for an opening to be formed during a planned surgical procedure. For example, a system 400, system 800, and/or user may calculate a set of predefined bone graft dimensions for an opening that is to receive a bone graft, including an autograft, the set of predefined bone graft dimensions may be unique to a patient, as described above. The opening may be formed as part of planned surgical procedure. In one embodiment, preoperative planning software may be used to review models of bones of a patient, one or more instruments for use in the surgical procedure, and a sequence of stages of a surgical procedure, including the formation of one or more openings between bones, bone fragments, joints, or the like.

Advantageously, a computing device operating, for example, preoperative planning software may automatically or by way of a user determine one or more predefined bone graft dimensions for an opening formed by one or more osteotomies of a surgical procedure. In one embodiment, predefined bone graft dimension may include a distance from a distal cut face of one bone to a proximal cut face of another bone on an opposite side of the opening. Said another way, one of the predefined bone graft dimensions may be measured between cut bone fragments of a model of bones and may be a distance or span across the opening along a straight line that is perpendicular to a surface of each bone fragment.

In certain embodiments, the computing device may calculate the predefined bone graft dimensions automatically. In another embodiment, the computing device may calculate the predefined bone graft dimensions in response to user input. For example, a user may identify a starting surface and an ending surface, and the computing device may automatically calculate a distance between the two surfaces. This distance may represent a thickness for an autograft bone graft that can be deployed within the opening. Alternatively, or in addition, the computing device may calculate a height and/or length of an opening based on a perimeter of each of the surfaces identified by a user. In this manner, the computing device may calculate a height, length, and/or depth of an opening between two bone fragments. Advantageously, the computing device can calculate the height, length, and/or depth for an autograft bone graft that is sized to fit satisfactorily into the opening between the bone fragments.

Those of skill in the art will appreciate that the set of predefined dimensions may be predefined dimensions for an opening that is associated with a surgical procedure. In one embodiment, the opening is formed by steps of the surgical procedure. In another embodiment, the opening may be a preexisting opening that a patient has when seeking the surgical procedure. The dimensions that can be included in the set of predefined dimensions includes, but is not limited to, each dimension that a computing device, either alone or with a user can measure, define, and/or calculate. For example, such dimensions may include a length, a thickness, a span length, a depth, a height, a width, a density, volume, surface area, a porosity, a luminosity, and the like.

Advantageously, one or more predefined bone graft dimensions can be used when designing and/or fabricating a harvesting guide. In addition, using a computing device and/or a model of bones for a surgical procedure and/or a modeled bone graft insertion site, a user, such as a surgeon can change the planned dimensions for a bone graft and this in turn may result in an adjustment to dimensions for features of a harvesting guide. In one embodiment, a surgeon may define a particular dimension for a bone graft to fill in resected bone and/or a joint space that is fused in a surgical procedure. In one embodiment, a surgeon may define a particular dimension for a bone graft to satisfy an objective that is different from filling in an opening formed in a surgical procedure. For example, a surgeon may require a certain height in order to harvest bone of a particular type and/or composition for one or more parts of a surgical procedure.

As also shown in FIG. 9, method 900 may include determining a position on a donor bone for a bone graft harvest site (block 904). For example, a system 400, system 800, and/or user may determine a position on a donor bone for a bone graft harvest site automatically or together based on user input. In one embodiment, a system, computing device, and/or user can reference anatomic data 412 about donor bone of a patient to be used for the bone graft harvest site. Alternatively, or in addition, a system, computing device, and/or user can reference a bone model generated based on medical imaging of the same bone of the patient. In this manner, the a system, computing device, and/or user can determine the dimensions of the donor bone and use these dimensions to recommend a position on the donor bone for the bone graft harvest site.

In certain embodiments, the position on a donor bone can be determined based on the type of bone and or composition of bone in or around the opening that the autograft will be deployed in. A surgeon, user, computing device, automated system, and/or semiautomated system, can determine a donor bone and/or a position on a donor bone for a bone graft harvest site.

For example, where the surgical osteotomy includes a Lapidus arthrodesis, the bones around the opening formed during the surgical procedure may include a distal end of the medial cuneiform and/or a cut proximal end of a base of the first metatarsal. These bones include a hard cortex or cortical surface around a softer internal part of the bone. The surgeon may prefer to deploy an autograft with a similar bone structure, a hard external cortex with softer internal bone. Consequently, a surgeon may seek an autograft from an area of the calcaneus where the dorsal cortex transitions to the lateral cortex.

Such an autograft is referred to as a bicortical graft. Those of skill in the art will appreciate that a surgeon may desire a tricortical autograft. In such embodiments, the tricortical autograft may be harvested from a hip bone of the patient. In certain embodiments, a surgeon may choose to harvest an autograft from a fibula to harvest a circumferential bone such as a fully cortical bone for use as an autograft. Thus, a surgeon, user, computing device, automated system, and/or semiautomated system may operate together and/or based on instructions from a surgeon regarding selection of a donor bone and/or determining a position on a donor bone for a bone graft harvest site.

As further shown in FIG. 9, method 900 may include developing a patient-specific harvesting guide model having a bone engagement surface configured to engage with a surface of the bone graft harvest site (block 906). For example, a system 400, system 800, and/or user may define a predefined depth/span/thickness (e.g., predefined span) for the opening that will receive an autograft bone graft. In one embodiment, the harvesting guide model may include a body, a set of bone attachment features, and a set of resection features (e.g., channels, cut channels, slots, holes, or other openings configured to receive or interact with a cutting tool). Advantageously, the set of resection features are positioned relative to each other such that performing one or more osteotomies using the resection features forms an autograft bone graft at the bone graft harvest site. Advantageously, the autograft bone graft has the same span/depth/thickness (also referred to as width) as the predefined span/depth/thickness (also referred to as width) for the opening that will receive an autograft bone graft. The patient-specific harvesting guide model may comprise a computer-aided design (CAD) model within a computing device.

Alternatively, or in addition, the patient-specific harvesting guide model may include other features that are part of, or cooperate to facilitate, tissue harvesting. In one embodiment, the patient-specific harvesting guide model may include a feature such as a bone attachment feature that is configured to facilitate attachment of the patient-specific harvesting guide model for a harvesting procedure. One example of a bone attachment feature is a hole that extends through the patient-specific harvesting guide model together with a fastener such as a K-wire. The hole can be sized to accept a fastener such as a K-wire. For example, in one embodiment, pin locations for temporary fasteners, such as K-wires may be defined in the patient-specific harvesting guide model and function as bone attachment features.

Alternatively, or in addition, the patient-specific harvesting guide model may also include one or more guards. Guards can serve to prevent resection beyond a boundary defined by the guards. In one embodiment, the guards may be implemented as fasteners such as pins, or guard pins.

As also shown in FIG. 9, method 900 may include fabricating a patient-specific harvesting guide based on the patient-specific harvesting guide model (block 908). For example, a system 400, system 800, and/or user may fabricate a patient-specific harvesting guide based on the patient-specific harvesting guide model, described above. In one embodiment, a computing device may send instructions for an additive manufacturing system to fabricate patient-specific harvesting guide based on the definition of the patient-specific harvesting guide model. Advantageously, the patient-specific harvesting guide includes the set of resection features that, when used, will provide an autograft bone graft having one or more dimensions that match the set of predefined bone graft dimensions.

For example, a span/depth/thickness (also referred to as width) for the autograft bone graft matches a predefined span/depth/thickness. The predefined span/depth/thickness, in certain embodiments, is defined using a computing device when the surgical procedure is being planned. For example, an opening defined after one or more osteotomies can be measured using the computing device to predefine (e.g., before the surgical procedure) a set of predefined dimensions for the opening. The set of predefined dimensions for the opening can then be used to determine one or more dimensions for an autograft bone graft planned to be secured within the opening. In certain embodiments, one or more dimensions for the autograft bone graft (e.g., thickness) may be increased beyond a span of the opening to accommodate bone loss resulting from cutting tool kerf.

In this manner, a surgeon can harvest the autograft bone graft with reduced stress or concern that the resulting autograft bone graft is too small or not the right shape. In addition, a surgeon is assured that the harvested autograft bone graft will be usable and not ruined due to an inaccuracy in harvesting the autograft bone graft.

Alternatively, or in addition, the patient-specific harvesting guide can be configured to fit in, or on, a single location on a donor bone. In this manner, the surgeon can be assured that the autograft bone graft will have the desired composition (e.g., a number of cortical surfaces, etc.). Alternatively, or in addition, the features and/or configuration of the patient-specific harvesting guide can facilitate resecting an autograft bone graft that meets one or more additional predefined bone graft dimensions (e.g., height, length, cross sectional diameter). As described herein, the patient-specific harvesting guide may be made using a variety of fabrication techniques and/or materials, including ceramic, plastic, and/or metal.

As further shown in FIG. 9, method 900 may include providing the patient-specific harvesting guide for a surgical procedure (block 910). For example, a system 400, system 800, and/or user may provide the patient-specific harvesting guide for a surgical procedure by sending the patient-specific harvesting guide to a surgeon, a facility hosting the surgical procedure, or the like. The patient-specific harvesting guide may be provided alone or together with one or more preoperative plans and/or one or more instruments (e.g., conventional instruments and/or patient-specific instruments). In certain embodiments, a technician, a laboratory, a factory, a fabrication facility, a vendor, and/or a medical services facility can provide the patient-specific harvesting guide for a surgeon.

Although FIG. 9 shows example blocks or steps of a process 900, in some implementations, A method 900 may include additional steps, fewer steps, different steps, or differently arranged steps than those depicted in FIG. 9. Additionally, or alternatively, two or more of the steps of a method 900 may be performed in parallel.

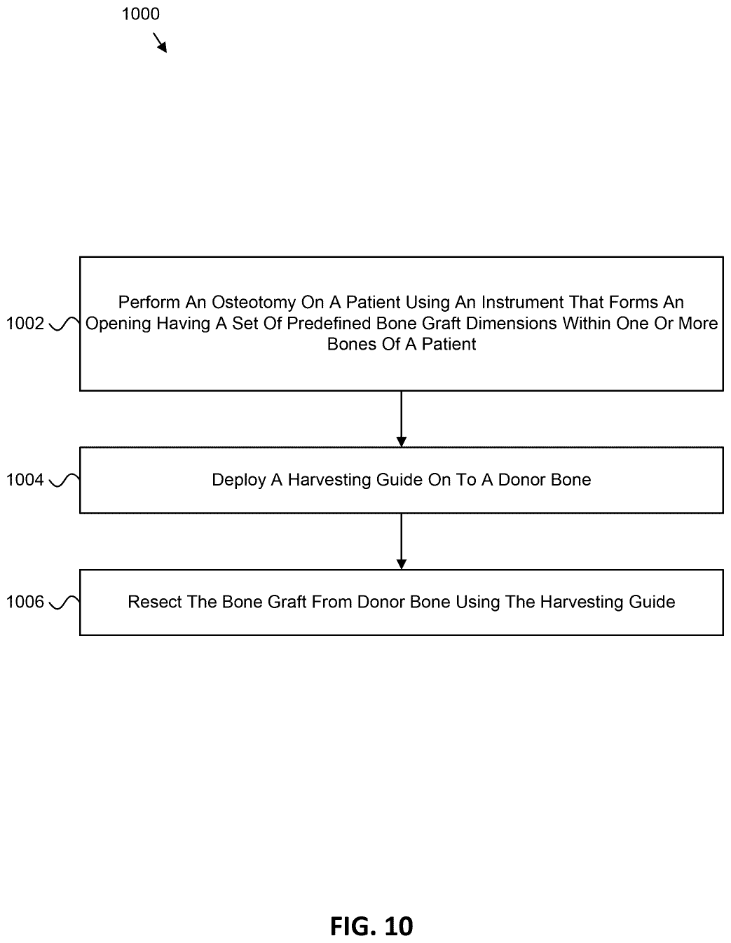

FIG. 10 is a flowchart of an example method 1000 for harvesting a graft, such as an autograft bone graft. In particular, the method 1000 can be used to harvest an autograft bone graft as part of a Lapidus arthrodesis. For example, the Lapidus arthrodesis may result in a toe being shortened due to the fusing of the joint. Advantageously, apparatuses, systems, and methods of the present disclosure can be used to provide an autograft bone graft a surgeon can position within an opening formed by resecting both bones of a tarsometatarsal (TMT) joint to mitigate potential shortening of the toe.

In some implementations, one or more method steps of FIG. 10 may be performed by a system 400, system 800, medical robot, and/or user. Those of skill in the art will appreciate that the present disclosure can be used to harvest a graft from a patient, from a donor bone such as from a cadaver, and/or a donor bone from a different species.

As shown in FIG. 10, method 1000 may include performing an osteotomy on a patient using an instrument that forms an opening having a set of predefined bone graft dimensions within one or more bones of a patient (block 1002). The osteotomy may be performed on a single bone or a plurality of bones of a patient (e.g., a medial cuneiform 202 and a first metatarsal 208). Where the osteotomy is on a single bone, a diseased or damaged part of the bone may be removed leaving an opening having a set of predefined dimensions. Alternatively, or in addition, the osteotomy may be on a single bone and form an opening that is to be filled with a bone graft larger than a span/depth/thickness of the opening to lengthen the bone.

In certain embodiments, performing the osteotomy may include a plurality of osteotomies performed on two or more bones. For example, in a Lapidus arthrodesis an osteotomy of the medial cuneiform 202 may be done and an osteotomy of the first metatarsal 208 may be done at a TMT joint. Often the osteotomies form a planar cut face on each bone. In situations where the surgeon desires the planar cut faces to remain parallel, a surgeon desires an autograft bone graft having two opposing parallel planar ends.

Normally harvesting an autograft bone graft having two opposing parallel planar ends by way of free hand cuts (no use of a guide) can be very challenging. Particularly, doing so such that the opposing ends are truly parallel. Advantageously, the present disclosure provides an embodiment of a harvesting guide that enables a surgeon to readily harvest an autograft having two opposing parallel planar ends of a least one dimension that substantially matches a dimension of a set of predefined dimensions for an opening formed by the osteotomies. This is accomplished at least in part by designing the harvesting guide in a computing device that has access to predefined dimensions of the opening.

In certain circumstances, a surgeon may desire a wedge-shaped autograft bone graft for insertion into an opening formed by one or more osteotomies. Advantageously, the same methods, apparatuses, and/or systems of the present disclosure can be used to design and/or fabricate a harvesting guide that facilitates harvesting a wedge-shaped autograft bone graft sized and/or configured to fit as desired within the opening formed by one or more osteotomies.

In one embodiment, the instrument is a cutting tool. In another embodiment, the instrument is a cutting tool together with a resection guide.

In one embodiment, the osteotomy may be a stage in a Lapidus arthrodesis. For example, the opening may be formed by removing part of a proximal base of a first metatarsal and/or part of a distal end (e.g., the articular surface) of a medial cuneiform. The opening may be defined as a space between where the first metatarsal 208 and medial cuneiform 202 are positioned before forming an osteotomy on each bone. In other words, the opening is the space between the bones when the bones were in their natural position relative to each other after the osteotomy removes a portion of the bone from one or the other of the two bones of the joint.

Advantageously, the present disclosure can be used to define a bone graft (e.g., in certain embodiments, an autograft bone graft) that fits as desired within the opening. Of course, a surgeon can determine and preplan how they want the autograft bone graft to fit within the opening. For example, the surgeon may want the autograft to fit loosely, snugly, or tightly. In some cases, a tight fit may be desired to ensure stability, but a surgeon may take care to size the autograft to avoid excessive compression, which could potentially cause pressure, undesired lengthening of a toe, or displacement of surrounding bones.

Advantageously, a surgeon can determine what shape and size is desirable for the autograft bone graft. In certain embodiments, the bone graft may be cylindrical and have two base sides connected by a curved lateral surface. In another embodiment, the bone graft may be wedge shaped and have fives sides and an edge (four sides that connect to the edge and one side opposite the edge). In one embodiment, the bone graft may have six sides. Using the systems, methods, and apparatus of the present disclosure a bone graft is designed that provides a desired fit within the opening. Alternatively, or in addition, embodiments according to the present enable a harvesting guide assists a surgeon in creating an autograft that has a desired number of sides and the sides are of a desired set of dimensions to suitably fit within the opening having a set of predefined dimensions.

In one embodiment, with an opening for a Lapidus arthrodesis, the dimensions of the opening may include a depth (also referred to as a span or thickness) measured between a cut face of the medial cuneiform 202 and a cut face of the first metatarsal 208 along an anterior-posterior axis (proximal-distal line of the foot), a height measured between a dorsal surface of one of the medial cuneiform 202 and the first metatarsal 208 and a plantar surface of one of the medial cuneiform 202 and the first metatarsal 208 along an cephalad-caudal axis, and a width measured between a lateral surface of one of the medial cuneiform 202 and the first metatarsal 208 and a medial surface of one of the medial cuneiform 202 and the first metatarsal 208 along an medial-lateral axis. In one embodiment, these dimensions are measured within a bone model using a computing device that defines the opening as part of a preoperative plan for a surgical procedure.

Of course, a height from a dorsal surface of a medial cuneiform 202 along a cut face of the medial cuneiform 202 to a plantar surface of the medial cuneiform 202 along the cut face may be different from a height from a dorsal surface of the first metatarsal 208 along a cut face of the first metatarsal 208 to a plantar surface of the first metatarsal 208 along the cut face. In certain embodiments, these two height may be averaged for the set of predefined dimensions for the opening. Alternatively, or in addition, the actual height of each cut face may be used as part of the predefined dimensions of the opening.

Similarly, a width from a lateral surface of a medial cuneiform 202 along a cut face of the medial cuneiform 202 to a medial surface of the medial cuneiform 202 along the cut face may be different from a width from a lateral surface of the first metatarsal 208 along a cut face of the first metatarsal 208 to a medial surface of the first metatarsal 208 along the cut face. In certain embodiments, these two widths may be averaged for the set of predefined dimensions for the opening. Alternatively, or in addition, the actual width of each cut face may be used as part of the predefined dimensions of the opening.

In another embodiment, each of these opening dimensions (e.g., depth/span/thickness, height, width) can have a different length along a side that will be a face of the autograft (implemented first as an autograft model). The opening may include a proximal end, a distal end, a dorsal end, a plantar end, a medial end, and a lateral end.

In one example, a height on a proximal end of the opening can be is substantially the same as a height of the cut face of the medial cuneiform, a height on a distal end of the opening can be substantially the same as a height of the cut face of the first metatarsal. Similarly, a depth along a medial end of the opening can be substantially the same as a length between a medial side of the cut face of the medial cuneiform and a medial side of the cut face of a first metatarsal, a depth along a lateral end of the opening can be substantially the same as a length between a lateral side of the cut face of the medial cuneiform and a lateral side of the cut face of a first metatarsal. Similarly, a width along a proximal end of the opening can be substantially the same as a length between a medial side of the cut face of the medial cuneiform and a lateral side of the cut face of the medial cuneiform and a width along a distal end of the opening can be substantially the same as a length between a medial side of the cut face of the first metatarsal and a lateral side of the cut face of the first metatarsal.

Those of skill in the art will appreciate that each of these six sides of the opening can be measured or calculated using a computing device and a bone model of the bones involved in the planned surgical procedure and each measurement can be used to define a dimension of a bone graft model that will fit within the opening. In certain embodiments, the bones of the model may be model bones of a patient for the surgical procedure. Alternatively, or in addition, one or more of these measurements/calculations may be increased or decreased beyond the corresponding dimension of the opening to provide a correction and/or adjustment to the anatomy of the patient.

As also shown in FIG. 10, method 1000 may include deploying a harvesting guide on to a donor bone (block 1004). For example, a system 400, system 800, and/or user may deploy the harvesting guide on to the donor bone. In certain embodiments, the harvesting guide may include one or more of the aspects, features and/or configurations of the embodiments of the harvesting guides described herein and/or covered by the claims included herewith. Specifically, the harvesting guide may include a first resection feature and a second resection feature. Further, the first resection feature and second resection feature may be configured relative to each other such that an osteotomy formed using the first resection feature and an osteotomy formed using the second resection feature facilitates resection of a bone graft having at least one dimension that substantially matches a dimension of the set of predefined dimensions. For example, in one embodiment, the harvesting guide may guide a surgeon in making two opposing parallel cuts in the donor bone. These may be the more difficult cuts for forming the autograft. A surgeon may then make one or more additional cuts without a harvesting guide to complete the formation and/or harvesting of the autograft bone graft.

In one embodiment, deployment may include deploying one or more fasteners that couple the harvesting guide to the donor bone. In one embodiment, the harvesting guide includes two resection features, each spaced away from each other such that the draft will have a dimension that substantially matches a dimension of the set of predefined bone graft dimensions.

In certain embodiments, the harvesting guide includes a bone engagement surface configured to engage with a surface of the donor bone at a bone graft harvest site. In one embodiment, the bone engagement surface is configured to engage at least two cortical surfaces of the donor bone. In certain embodiments, the two cortical surfaces are adjacent such as: all or a portion of a lateral surface and a dorsal surface, all or a portion of a dorsal surface and a medial surface, all or a portion of a lateral surface and a planter surface, all or a portion of a plantar surface and a medial surface, and the like. In another embodiment, the bone engagement surface is configured to engage at least three cortical surfaces of the donor bone (e.g., all or a portion of a plantar surface, lateral surface, and a dorsal surface,).

Cortical bone sections for a bone graft may be desirable because of their increased strength versus medullary bone. Thus, the deployment 1004 of the harvesting guide may include positioning and/or adjustment of a position of the harvesting guide until the bone engagement surface registers to one or more surfaces of the donor bone. Advantageously, registration of the harvesting guide with the donor bone provides assurance to a surgeon that the harvesting guide is positioned in the same or substantially the same position as in the preoperative plan developed using the computing device.

As further shown in FIG. 10, method 1000 may include resecting the bone graft from the donor bone using a first resection feature and/or a second resection feature of the harvesting guide to form the bone graft (block 1006). For example, a system 400, system 800, and/or user may perform one or more osteotomies by cutting the bone with a cutting tool inserted into one or more of the first resection feature and the second resection feature to resect the bone graft. Of course, a surgeon can decide how much to use the harvesting guide. For example, a surgeon may use each of the resection features of the harvesting guide. Alternatively, the surgeon may use one of a plurality of resection features to form one of the osteotomies in the donor bone. Next, the surgeon may perform other osteotomies to form the bone graft without the harvesting guide.

Method 1000 may include removing/resecting the bone graft from the donor bone. In one embodiment, performing osteotomies using one or more resection features may separate the graft from the donor bone (e.g., cutting three sides that separates the bone graft). In other embodiments, performing osteotomies using one or more resection features may form one or more of the more difficult osteotomies, specifically, those that are most difficult to achieve without a guide (e.g., cutting two or three sides of four that need to be cut to separate the bone graft). In certain embodiments, after resection using one or more resection features, the graft may still be connected to the donor bone, in such a embodiments, a surgeon may use another tool such as an osteotome or other cutting tool (without a harvesting guide) to separate the graft from the donor bone.

Although FIG. 10 shows example blocks or steps of a process or method 1000, in some implementations, a method 1000 may include additional steps, fewer steps, different steps, or differently arranged steps than those depicted in FIG. 10.

Additionally, or alternatively, two or more of the steps of a method 1000 may be performed in parallel. For example, in one embodiment, the method 1000 may further include preparing the bone graft for reduction within the opening; and reducing the graft and at least one bone fragment within the opening. Preparing the bone graft may include cutting or shaping the bone graft to a desired size and/or shape.

Preparing the bone graft may also include using a jig and a cutting tool to shape and/or resize the bone graft. Advantageously, the jig may be configured to guide shaping of a bone graft resected using the harvesting guide. For example, the jig may include an opening that is sized and shaped to match the predefined bone graft dimensions either of the opening and/or of a desired set of predefined bone graft dimensions for the surgical procedure. Like the harvesting guide, the jig may be modeled first and/or designed using a computing device to a desired configuration and then fabricated for use in the surgical procedure.

Reducing at least one bone fragment with the bone graft within the opening may be part of deploying the bone graft within the opening.

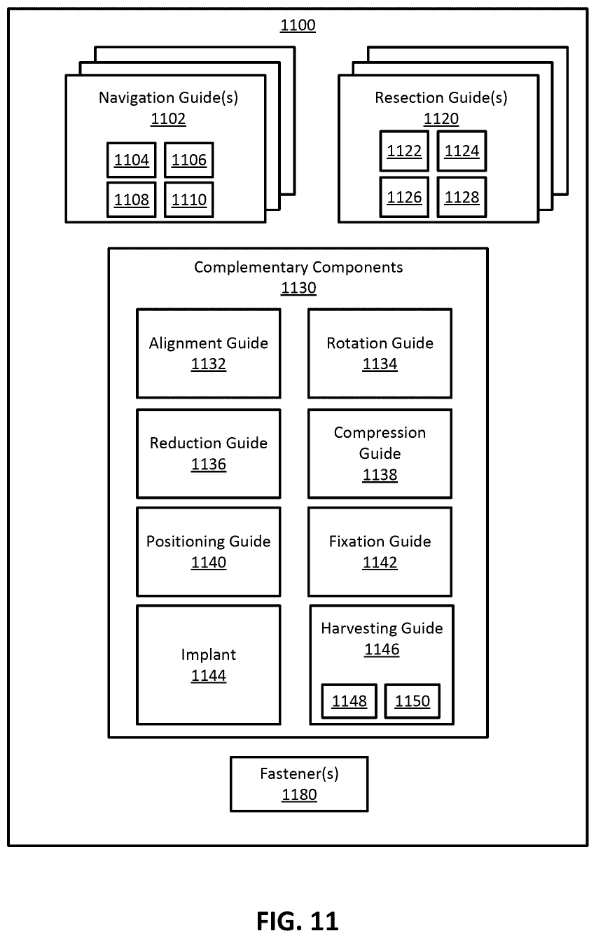

FIG. 11 illustrates an exemplary system 1100, according to one embodiment. The system 1100 may include one or more navigation guides 1102, one or more resection guides 1120, one or more complementary components 1130, and one or more fasteners 1180. While a system 1100 can be used for a variety of procedures, one or more features, components, and/or aspects of the system 1100 may be particularly suited for one or more osteotomies on one or more bones of a patient's anatomy such as a patient's foot, ankle, wrist, hand, shoulder, or the like. In certain embodiments, the system 1100 may not include a navigation guide 1102.

A navigation guide 1102 can serve to guide a surgeon during one or more stages of a surgical procedure. In one embodiment, the navigation guide 1102 can provide a guide or a template for a surgeon for where to form, provide, position, and/or orient one or more reference features.

In the present disclosure, a reference feature(s) (also referred to as anatomical references) can provide an intraoperative feature that can enable a surgeon to model patient anatomy, model instrumentation, model implants, and/or practice a surgical procedure either virtually or using physical models, and then during the surgery configure, position, orient and/or register instrumentation, implants, and/or other surgical components to perform the surgery using the registered/positioned instrumentation, implants, and/or other surgical components developed, designed, and/or refined in a virtual or simulated procedure. Within a computer model and/or a three-dimensional physical model, a user can define one or more model references. A reference feature corresponds to a particular model reference. In one example embodiment, a reference feature can be realized by a hole, tunnel, or other opening, in a bone or other hard tissue and/or in soft tissue. In another example embodiment, a reference feature can be realized by a hole, tunnel, or other opening, in a bone or other hard tissue and/or in soft tissue together with another structure such as a fastener such as a bone screw, a K-wire, or the like. Reference features map a model reference from a virtual environment to a physical environment.

Advantageously, the navigation guide 1102 facilitates providing, forming, establishing, and/or configuring one or more reference features for a surgical procedure. The navigation guide 1102 can include a body 1104, an opening 1106, and one or more position indicators 1108. In certain embodiments, a navigation guide 1102 may include a bone attachment feature 1110. In such embodiments, the bone attachment feature 1110 can be used to secure the navigation guide 1102 to a bone or bone fragment, at least temporarily.

The one or more resection guides 1120 assist a surgeon in performing one or more different resection steps for an osteotomy procedure. In certain embodiments, a resection guide 1120 includes one or more resection features 1122 and one or more bone attachment features 1124. The resection features 1122 can take a variety of forms and/or embodiments. Similarly, the bone attachment features 1124 can take a variety of forms and/or embodiments. The bone attachment features 1124 may be similar to, the same as, or different from, the bone attachment feature 1110 that can be used with the navigation guide 1102. In one embodiment, a resection guide 1120 may include a bone attachment feature 1124 that is at an oblique angle relative to one or more anatomical references. The obliquely angled bone attachment feature 1124 may provide more stability for the resection guide 1120 during a resection.

The resection features 1122 provide a guide for a surgeon using a cutting tool to resect a bone, one or more bones, or other tissues of a patient. The bone attachment features 1124 serve to secure the resection guide 1120 to one or more bones, one or more bone fragments, and/or one or more other structures. In one example, a bone attachment feature 1124 can include a hole in the resection guide 1120 together with a temporary fastener such as a K-wire or pin.

The bone attachment features 1124 can be used to engage or connect or attach a resection guide 1120 to one or more bones, or bone fragments, of a patient. The bone attachment features 1124 may include any of a wide variety of fasteners including, but not limited to, holes, prongs, spikes, fastening devices, and/or the like. Effective engagement or connection of the resection guide 1120 to one or more bones along a single bone, across a single joint, across a plurality of joints, or the like, can ensure that cut surfaces are formed in desired locations and orientations and mitigate removal of hard tissue and/or soft tissue in undesired locations.

In certain embodiments, a resection guide 1120 may include one or more bone engagement members that may be embodied as one or more bone engagement surfaces 1126 and/or one or more landmark registration features 1128. In certain embodiments, a landmark registration feature 1128 may extend from one or more sides of the resection guide 1120 and engage with one or more landmarks of a bone of a patient. Registration of the landmark registration feature 1128 to the landmark of the bone can serve to confirm that a surgeon has located a desired placement and/or orientation for a resection guide 1120. In certain embodiments, the 73avigateon guide 1102 can include one or more bone engagement surfaces 1126 on a surface of the navigation guide 1102 that faces a bone or bone fragment. In other embodiments, a resection guide 1120 may include no bone engagement surface 1126 or landmark registration feature 1128.

In certain embodiments, the bone engagement surfaces 1126 are patient-specific: contoured to match a surface of one or more bones the resection guide 1120 contacts during the procedure. Alternatively, or in addition, the bone engagement surface 1126 may not be patient-specific and may, or may not, contact a bone surface during use of the resection guide 1120. Those of skill in the art appreciate that one or more sides of any of the members of the system 1100 may include one or more bone engagement surfaces 1126. Consequently, one or more sides of the navigation guide 1102, the resection guide(s) 1120, the complementary components 1130, fasteners 1180, and/or the implants 1144 may include one or more bone engagement surfaces 1126.

Alternatively, or in addition, the resection guide 1120 may be selected from a kit, collection, or repository of a number of resection guides 1120: each having a different configuration for resecting one or more parts of one or more bones. For example, each member of the repository/kit may include a one or more numbers. A first number may indicate a number of millimeters a specific resection feature of the resection guide 1120 will remove and a second number may indicate a number of millimeters a another resection feature of the resection guide 1120 will remove. For example, a resection guide 1120 may include a +1 near a proximal end and a +1 near a distal end of the resection guide 1120 (indicated+1:+1) and may include a proximal end resection feature and a distal resection feature. In such an example, the resection guide 1120 will remove 1 millimeter from distal end of a medial cuneiform 202 and 1 millimeter from proximal end of a first metatarsal 208. Accordingly, a kit may include resection guide 1120 with configurations such as +2:+1, or +1:+3, or the like. In certain embodiments, the kit may include resection guide 1120 configurations such as +0:+1, or 0:+3 where the 0 indicates that the resection will be done according to a preoperative plan developed using the model and the + number indicates an additional amount of tissue to be resected beyond the amount planned for in the preoperative plan.

Having a plurality of different resection guides 1120 can be advantageous where a surgeon sees something intraoperatively that causes them to change the preoperative plan. Often, a surgeon seeks to minimize the amount of tissue removed during a surgical procedure and to create a resected bone surface that will support an effective union and the preoperative plan also works towards these goals. However, during the operation a surgeon may alter the plan based on what they encounter and thus may opt to use a different resection guide 1120 than was originally planned. Advantageously, the present disclosure facilitates this.

Alternatively, or in addition, a resection guide 1120 may be configured to resect one or the other of the bones of a joint.

The complementary components 1130 can serve to assist a surgeon during one or more steps of a procedure. Those of skill in the art appreciate that a number of components can serve as complementary components 1130. One or more of the features, functions, or aspects of the complementary components 1130 can include patient-specific features.

Examples of complementary components 1130 include, but are not limited to, an alignment guide 1132, a rotation guide 1134, a reduction guide 1136, a compression guide 1138, a positioning guide 1140, a fixation guide 1142, one or more implants 1144 and/or a harvesting guide 1146. In general, the complementary components 1130 serve to assist a surgeon in performing the function included in the name of the complementary component 1130. Thus, an alignment guide 1132 can help a surgeon align bones, parts of bones, anatomical body parts, or other parts of a patient as part of a procedure. A rotation guide 1134 can help a surgeon rotate one or more bones, parts of bones, or other body parts of a patient as part of a procedure. A harvesting guide 1146 can help a surgeon harvest a graft.

A reduction guide 1136 can help a surgeon position and/or orient one or more bones, bone fragments, or other parts of a patient as part of a procedure in order to reduce the bone, bones, bone fragments, or other parts and/or in order to position and/or orient the bone, bones, bone fragments, or other parts to a desired position and/or orientation. A compression guide 1138 can help a surgeon compress one or more bones, bone fragments, or other parts of a patient together or against an implant as part of a procedure. A positioning guide 1140 can help a surgeon position one or more bones, parts of bones, other parts of a patient, instruments, or other structures as part of a procedure. One example of a positioning guide 1140 is a positioner, described above.

A fixation guide 1142 can help a surgeon in completing one or more temporary or permanent fixation steps for one or more bones, parts of bones, or other parts of a patient as part of a procedure. The fixation guide 1142 may include and/or may use one or more components of a fastener or fixation system including implant hardware of the fastener or fixation system.

One example of a complementary components 1130 may include a compressor/distractor, which is one example of a compression guide 1138. The compressor/distractor can be used to compress and/or distract bones or parts of bones involved in a procedure.

Advantageously, the system 1100 can help a surgeon overcome one or more of the challenges in performing an osteotomy procedure and/or harvesting a graft, particularly on bones of a hand or of a foot of a patient, such as on the forefoot, midfoot, or hindfoot. One challenge during an osteotomy procedure can be maintaining control of, and/or position, and/or orientation of a bone, one or more bones, and/or bone pieces/fragments, particularly once a resection or dissection is performed. Advantageously, the navigation guide 1102, resection guide(s) 1120, and/or complementary components 1130 can be configured to assist in overcoming this challenge.

Advantageously, the system 1100 can help a surgeon in positioning, placing, and/or orienting a instruments for the procedure accurately. Modern techniques may include preoperative planning, simulation, or even practice using computer models, 3D printed models, virtual reality systems, augmented reality systems or the like. However, simulations and models are still different from actually positioning a one or more instruments on a patient's bone, joint, or body part during the procedure. The system 1100 can include a number of features, IIing for example the reference features, patient-specific features (which can include reference features), to assist the surgeon with the positioning.

Advantageously, the system 1100 can help a surgeon in securing guides of the osteotomy system 1100, as well as how to readily remove the instrumentation without disturbing a reduction, shifting, reorienting, or repositioning one or more bones or parts of bones while removing the guide. In certain embodiments, the system 1100 is configured to permit removal of instrumentation while keeping temporary fasteners in place for use in subsequent steps of an osteotomy procedure. Alternatively, or in addition, the system 1100 facilitates positioning of temporary or permanent fasteners during one step of the osteotomy procedure for use in a subsequent step of the osteotomy procedure. For example, holes or openings formed in the bone during one step of the osteotomy procedure can serve as pilot or starter holes for subsequent permanent fasteners and/or other hardware. Removal of instrumentation during an osteotomy procedure can be particularly challenging where translation and/or rotation of the bones involved in the osteotomy procedure is required for the success of the osteotomy procedure. Advantageously, the system 1100 accommodates translation and/or rotation of the bones during the osteotomy procedure while facilitating a successful outcome for the osteotomy procedure.

Advantageously, the components of the system 1100 can be specifically designed for a particular patient. Alternatively, or in addition, the components of the system 1100 can be specifically designed for a class of patients. Each of the components of the system 1100 can be designed, adapted, engineered and/or manufactured such that each feature, attribute, or aspect of the component is specifically designed to address one or more specific indications present in a patient. Advantageously, cuts made for the osteotomy procedure can be of a size, position, orientation, and/or angle that provides from an optimal osteotomy and/or outcome with minimal risk of undesirable resection. In one embodiment, the components of the system 1100 can be configured such that an osteotomy is performed that enables a correction in more than one plane in relation to the parts of the body of the patient. For example, cut channels a resection guide 1120 can be oriented and configured such that when the bones are fused/fixated the correction results from translation, rotation, and/or movement of bones or bone parts in two or more planes (e.g., sagittal and transverse).

In one embodiment, the system 1100 can include a harvesting guide 1146, a jig 1148, and a guard 1150. The harvesting guide 1146 may include a body; a bone attachment feature; a first resection feature, the first resection feature configured to guide an osteotomy of a donor bone; and a second resection feature, the second resection feature configured to guide an osteotomy of a donor bone, the first resection feature extending through the body parallel to the second resection feature, the first resection feature separated from the second resection feature by a distance defined based on a patient-specific bone graft insertion site. In certain embodiments, the step 110 can also include a guard 1150.

The harvesting guide 1146 can serve to clearly identify the location for harvesting a bone graft and guide one or more resection cuts made to form a graft from the donor bone. Those of skill in the art will appreciate that the harvesting guide 1146 can be configured to facilitate resection of a graft of various sizes, shapes, and/or configurations.

The guard 1150, also referred to as a stop, is an apparatus, device, or structure that serves to assist a surgeon by preventing resection, dissection, or cutting of parts of tissue where resection is unplanned or undesired. Said another way, the guard prevents resection by a cutting tool beyond a boundary. In certain embodiments, the boundary is a line, plane, or point. In one embodiment, the boundary may be predefined using a computing device and/or a model of bones of a patient and/or one or more models of instruments to be used in a surgical procedure on the patient.

Advantageously, the harvesting guide 1146 can include holes that cooperate with one or more fasteners to serve as a guard 1150. The fastener can be made of a rigid and hard material such as metal that cannot be cut, or is not readily cut, by a surgical cutting tool. Once the fastener is deployed, the fastener serves as a guard 1150 or cutting boundary to prevent resection of a donor bone through, or beyond, the position of the fastener. Advantageously, the fastener can serve as a fastener for a bone attachment feature, as a trajectory guide, and/or as a guard 1150. The features and advantages of using the system 1100 can greatly reduce stress or pressure a surgeon may experience in performing graft harvesting. In addition, the harvesting guide 1146 and/or guard 1150 can facilitate smooth and efficient and prompt execution of the surgical procedure and may improve outcomes from the surgical procedure.

In certain embodiments, the one or more fasteners 1180 can include both, one or more permanent fasteners and one or more temporary fasteners. Typically, the fasteners 1180 may be used during a variety of different steps of a procedure. Temporary fasteners are often used because they can securely hold bone or bone fragments while steps of the procedure are conducted. A common temporary fastener that can be used with system 1100 is a K-wire, also referred to as a pin.

In certain embodiments, the exemplary system 1100 may include a plurality of navigation guides 1102, resection guides 1120, complementary components 1130, and/or fasteners 1180. For example, a surgeon may plan to resect a plurality of wedge segments from one or more bone(s) to accomplish a desired correction. One or more wedge segments may be resected from a medial side of a patient's foot and another one or more wedge segments may be resected from a lateral side of the patient's foot. These wedge segments may extend part way into the foot, or through from one side of the foot to the other. Of course, multiple wedge segments may be formed on one side of the foot as well.

In certain embodiments, the components of the system 1100 may be made as small as possible to minimize the amount of soft tissue that is opened or disturbed in the patient for the osteotomy procedure. Alternatively, or in addition, walls and/or sides of the components may be beveled and/or angled to avoid contact with other hard tissue or soft tissues in the operating field for the osteotomy procedure and/or to facilitate handlings and positioning by a user.

Those of skill in the art will appreciate that for certain osteotomy procedures a particular complementary component 1130 may not be needed or a particular complementary component 1130 may be optional for use in the osteotomy procedure. Similarly, those of skill in the art will appreciate that certain features of the navigation guide 1102, resection guides 1120, complementary components 1130, fasteners 1180 can be combined into one or more of apparatus or devices or may be provided using a plurality of separate devices.

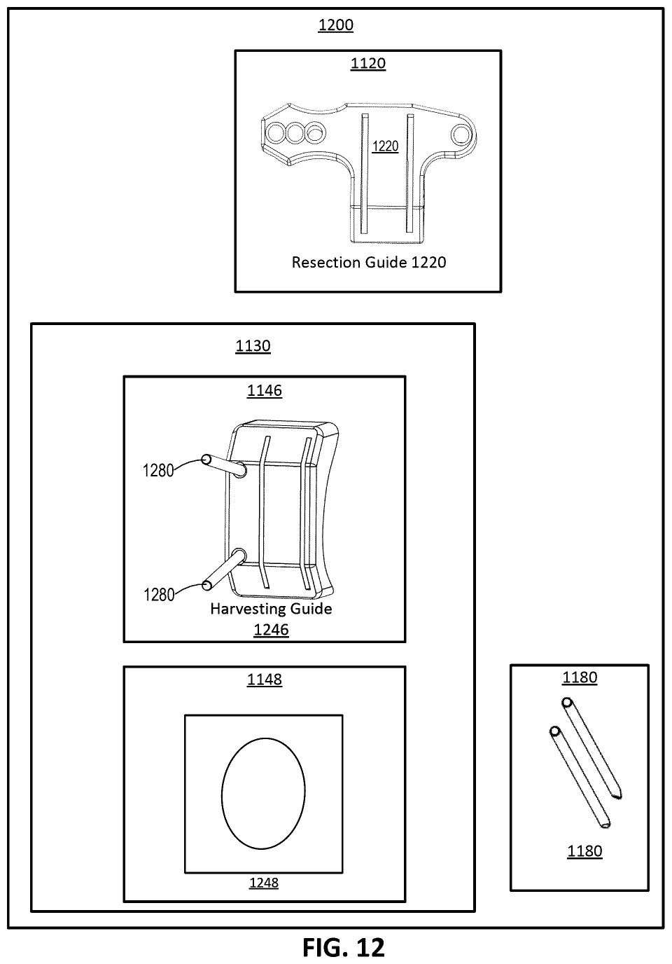

FIG. 12 illustrates an exemplary osteotomy system 1200 for harvesting bone for a surgical procedure, according to one embodiment. The osteotomy system 1200 may include one or more resection guides 1120, one example of which is resection guide 1220, one or more other complementary components 1130, such as a harvesting guide 1146, one example of which is harvesting guide 1246 and/or a jig 1148, one example of which is jig 1248, and one or more fasteners 1180. The osteotomy system 1200 can be used for a variety of surgical procedures.

The osteotomy system 1200 includes resection guide 1220. The resection guide 1220 facilitates resection of hard tissue and/or soft tissue of a patient for a surgical procedure. In one embodiment, the resection guide 1220 can be a standalone, separate apparatus. In another embodiment, the resection guide 1220 can be an apparatus that couples to, integrates with, and/or cooperates with a navigation guide 1202 to assist a surgeon in resection of patient tissue. In certain embodiments, the resection guide 1220 is patient-specific.

The osteotomy system 1200 includes a plurality of complementary components 1130. The osteotomy system 1200 includes harvesting guide 1246 that facilitates performing one or more osteotomies in bone of a donor bone to form a graft, in particular an autograft. The harvesting guide 1246 may include a bone attachment feature, a bone engagement surface, and/or one or more resection features. In certain embodiments, the harvesting guide 1246 is patient-specific.

In certain embodiments, the one or more fasteners 1180 can include one or more permanent fasteners and/or one or more temporary fasteners. Typically, the fasteners 1180 may be used during a variety of different steps of a procedure. Temporary fasteners are often used because they can securely hold bone or parts/fragments of bones while steps of the procedure are conducted. A common temporary fastener that can be used with osteotomy system 1200 is a K-wire, also referred to as a pin or guide pin.

The osteotomy system 1200 includes a harvesting guide 1246. The harvesting guide 1246 includes a body 1250 that includes a front side 1252, a back side 1254, a top side 1256, a bottom side 1258, a left side 1260, and a right side 1262. The harvesting guide 1246 also includes at least one resection feature 1264, 1266 that extends from one side of the body 1250 to an opposite side of the body 1250. The at least one resection feature may be referred to as a first resection feature 1264. The resection feature 1264 facilitates making one or more osteotomies in the donor bone. The first resection feature 1264 is configured to guide a first osteotomy of a donor bone. The harvesting guide 1246 may also include a second resection feature 1266 that extends from one side of the body 1250 to an opposite side of the body 1250. In one embodiment, the second resection feature 1266 is configured to guide a second osteotomy of a donor bone. In one embodiment, the second resection feature 1266 extends through the body 1250 parallel to the first resection feature 1264. Alternatively, the first resection feature 1264 and second resection feature 1266 may extend from the body 1250 at an angle such that cuts in the donor bone using the resection features 1264,1266 form a wedge-shaped bone graft. The harvesting guide 1246 also includes a bone attachment feature 1268 and, in certain embodiments, a bone engagement surface 1270 and/or a bone engagement member.

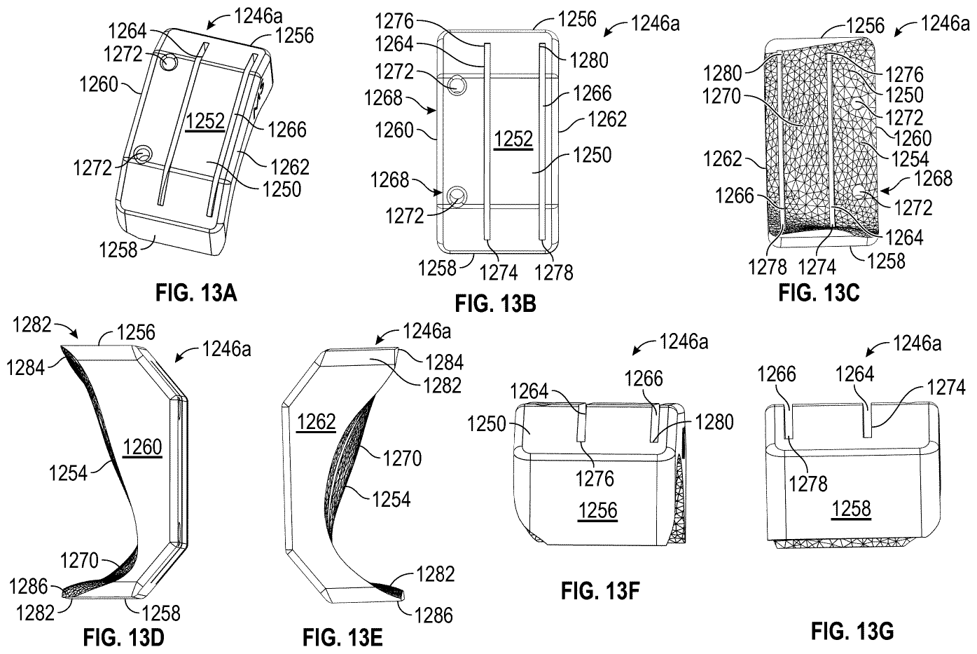

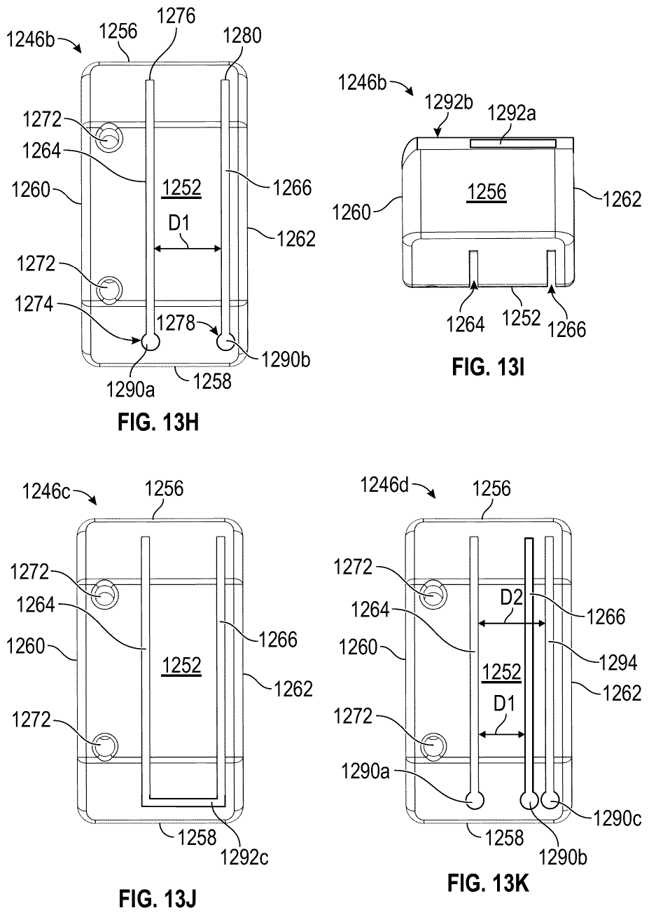

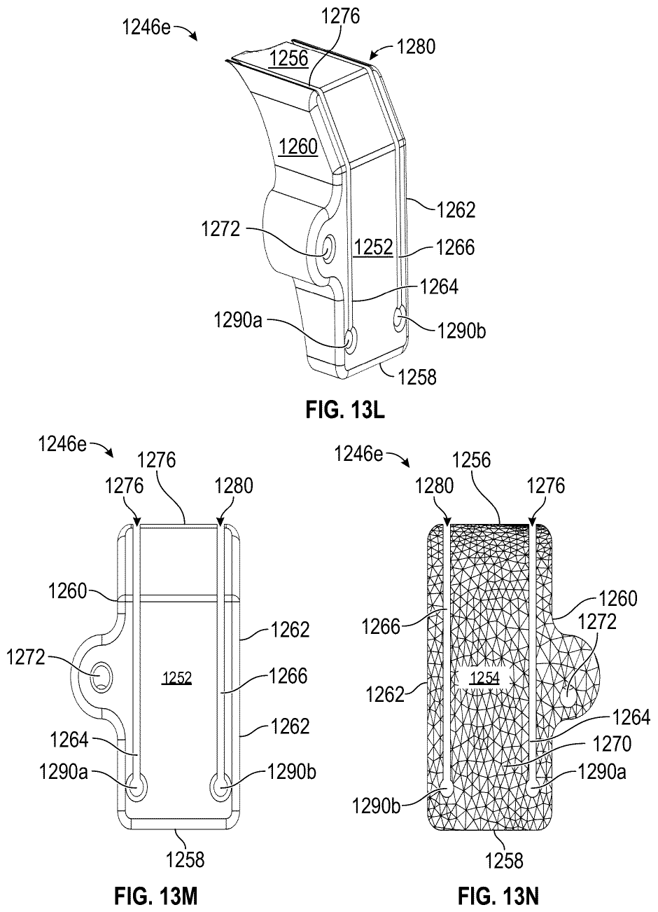

FIGS. 13A-13G are front perspective, front, back, left, right, top, and bottom views respectively, of a harvesting guide 1246a according to one embodiment. The harvesting guide 1246a includes a body 1250, a front side 1252, a back side 1254, a top side 1256, a bottom side 1258, a left side 1260, and a right side 1262, at least one resection feature 1264,1266, a bone attachment feature 1268 and/or, optionally, a bone engagement surface 1270.

The body 1250 provides the structural integrity for the harvesting guide 1246a. The body 1250 can be of a variety of shapes and sizes. The size, shape, and configuration of the harvesting guide 1246a may be determined by the surgical harvesting procedure the harvesting guide 1246a will be used with, by the preferences of a surgeon, by patient-specific features of a patient, a combination of these factors, or the like.

In certain embodiments, the bone attachment feature 1268 may be embodied as an opening 1272 sized, shaped, and configured to accept K-wire or pins or other fasteners 1180 or a drill bit. In one embodiment, the bone attachment feature 1268 includes an opening 1272 and a fastener 1180 deployed within the opening 1272. In certain embodiments, the harvesting guide 1246a is patient-specific: fabricated, designed, and/or contoured for the needs of a specific patient and/or preferences of a surgeon.

In one embodiment, an osteotomy system according to the present disclosure may include a plurality of harvesting guides 1246a (e.g., in a kit). Each harvesting guide 1246a of the plurality may have a different set of configurations, positions, angles, and/or features that may be patient-specific and/or may be generic, and/or meet a surgeon's preferences. The plurality of positioning guides 1240 can be used intraoperatively by a surgeon for a surgical procedure (e.g., bone graft harvesting surgical procedure).

In one embodiment, the body 1250 may be as small as possible to serve its function and still be readily handled by a user. In certain embodiments, a body 1250 can include one or more bevels that can facilitate handling and positioning of the harvesting guide 1246a by a user.

In another embodiment, the body 1250 may be transparent or at least transparent to medical imaging (e.g., radiolucent). The harvesting guide 1246a may include or be configured to accept one or more position indicators. At least one position indicator is positioned, configured, and/or arranged to indicate a position of the harvesting guide 1246a in relation to an anatomical reference and/or anatomical structure. For example, the position indicator may indicate a position of the harvesting guide 1246a relative to a joint (e.g., anatomical reference) of a patient.

In one embodiment, the harvesting guide 1246a may include one or more openings 1272 that extend into the body 1250. The one or more openings 1272 are configured to accommodate one or more fasteners 1180. In the illustrated embodiment, the one or more openings 1272 may be embodied as passages that extend from one side of the body 1250 to the other.

In certain embodiments, an osteotomy system includes the harvesting guide 1246a and the harvesting guide 1246a includes one or more features that can be used to provide, determine, deploy, install, configure, and/or establish at least one reference features. The at least one reference feature can serve as an interface between an instrument used for a surgical procedure and a bone or a bone fragment of a patient. Those of skill in the art will appreciate that a reference feature may be implemented and/or embodied in a variety of different ways and/or with a variety of different apparatus, devices, structures, and/or systems, each of which is considered within the scope of the present disclosure.

In the illustrated embodiment, the at least one reference feature can be embodied as a hole or opening in one or more bones and/or one or more bone fragments. In another embodiment, the at least one reference feature can be embodied as a protrusion or other structure (e.g., a pin, post, bone screw, etc.) connected to or engaged with one or more bones and/or one or more bone fragments.

Advantageously, the harvesting guide 1246a can be used to provide one or more different types of reference features. In one embodiment, the harvesting guide 1246a includes one or more openings or holes that can serve to provide a reference feature (either or both holes in bone and/or posts or protrusions that extend from bone). The one or more openings or holes may extend from a front side 1252 to a back side 1254 of the harvesting guide 1246a.

FIG. 13A is a perspective view of one embodiment of a harvesting guide 1246a. FIG. 13B is a front view of one embodiment of a harvesting guide 1246a. A first resection feature 1264 may include a first end 1274 and a second end 1276. A second resection feature 1266 may include a third end 1278 and a fourth end 1280. In one embodiment, the first resection feature 1264 is configured to guide an osteotomy (e.g., a first osteotomy) of a donor bone. The second resection feature 1266 is configured to guide an osteotomy (e.g., a second osteotomy) of a donor bone. One of the first end 1274 and the second end 1276 are spaced from one of the third end 1278 and the fourth end 1280 by a distance defined based on a patient-specific bone graft insertion site. In the illustrated embodiment, the first end 1274 is spaced from the third end 1278 by a first predefined distance. The second end 1276 is spaced from the fourth end 1280 by a second predefined distance. In one embodiment, the first predefined distance and the second predefined distance are the same. Alternatively, or in addition, the first predefined distance and the second predefined distance may be different. In one embodiment, the first end 1274 and the third end 1278 and second end 1276 and the fourth end 1280 are each spaced from each other by the same distance.

Referring to FIG. 13B, in one embodiment, a first resection feature 1264 and a second resection feature 1266 each extend through the body 1250 from a front side 1252 (e.g., non-bone-facing side) to a back side 1254 (e.g., bone-facing side). The first resection feature 1264 extends through the body parallel to the second resection feature 1266. In addition, both the first resection feature 1264 and the second resection feature 1266 are configured in this embodiment with a trajectory that intersects a surface of the donor bone at a perpendicular angle.

FIG. 13B illustrates that the position of the first resection feature 1264 and second resection feature 1266 relative to each other in the body may be offset from each other by a distance. This distance may be a length along the front side 1252 between the first resection feature 1264 and the second resection feature 1266. In certain embodiments, this distance may be defined by, and/or based on, a patient-specific bone graft insertion site. This distance may be an offset and may be defined by, and/or based on, a dimension that satisfies a predefined bone graft dimension.

In one example, the predefined bone graft dimension may be a depth, span, and/or thickness defined for an opening and/or a patient-specific bone graft insertion site. In certain cases, a predefined bone graft dimension for an opening may include a distance from a proximal end of the opening to a distal end of the opening. The proximal end being adjacent to a medial side of the opening and to a lateral side of the opening and the distal end being adjacent to the medial side of the opening and to the lateral side of the opening. Depending on the perspective used to view and/or measure this distance, in certain contexts and/or embodiments, this distance may be referred to as a width. For example, if an opening in bone is viewed from a lateral and/or medial side of the bones on a distal end and a medial end of the opening, the space between the bones may be referred to as a width. In general, this space is referred herein as a span, depth, or thickness of a bone graft. Thickness or span are useful to describe the magnitude of the distance between two bones when viewed perpendicular to a longitudinal axis of one or both bones.

In certain embodiments, the distance that first resection feature 1264 is offset from second resection feature 1266 within the body 1250 may be the same or greater than a predefined bone graft dimension for a planned opening between bones or bone fragments as part of a surgical procedure. Alternatively, or in addition, the distance that first resection feature 1264 is offset or separated from second resection feature 1266 within the body 1250 may be the same or greater than a depth/span/thickness dimension for a planned opening between bones or bone fragments as part of a surgical procedure. Alternatively, or in addition, the distance that first resection feature 1264 is offset or separated from second resection feature 1266 within the body 1250 may be the same or greater than a depth/span/thickness dimension for a planned autograft bone graft to be harvested for use in a surgical procedure.

Advantageously, a surgeon can use dimensions and/or measurements from a planned surgical procedure to define one or more dimensions of a bone graft to be harvested using a harvesting guide fabricated based on those one or more dimensions. In this manner, a surgeon can be assured that a suitable bone graft can and will be harvested that will provide a desired outcome.

In certain embodiments, a distance between first resection feature 1264 and second resection feature 1266 along the surface front side 1252 can be defined by a dimension for an opening in a planned opening for a surgical procedure and/or a dimension (e.g., a depth/span/thickness dimension) for a model bone graft that is planned to be inserted between two bone fragments. Alternatively, or in addition, a surgeon may make the distance slightly larger than a predefined bone graft dimension to account for a thickness of a blade or bit of a cutting too and/or to account for kerf bone material created during the osteotomy.

In the illustrated embodiment, the resection feature 1264 and resection feature 1266 may be parallel to each other for forming a block shaped autograft (e.g., a cylindrical shaped autograft having two planar opposing sides and a circular lateral side between the two planar opposing sides). In this manner, an autograft can be formed that has a uniform thickness between two planar opposing sides/cut faces. Having an autograft can help ensure that the autograft provides a filling and/or lengthening function without changing a trajectory of the bones fused to the autograft. This can be helpful to a surgeon because other aspects of a surgical procedure may have been specifically planned to correct or change a bone trajectory. Thus, a uniform thickness of the autograft does not interfere with those plans.

Alternatively, or in addition, the resection feature 1264 and/or resection feature 1266 and one or more other resection features may be included so that practically any shape autograft can be formed from the donor bone. For example, the resection feature 1264 and resection feature 1266 may be angled towards each other on one end or the other or both ends or on a distal end of the resection features near a back side 1254 of the harvesting guide 1246a. Those of skill in the art will appreciate that the position and/or orientation and/or angles of the resection features can be defined to form a three-dimensional autograft that includes three planes and/or include three cortical surfaces of bone of a donor bone.

Those of skill in the art will appreciate that a distance that satisfies a predefined bone graft dimension can include a variety of relationships between the distance and the predefined bone graft dimension. In one embodiment, a distance that satisfies a predefined bone graft dimension can mean that the distance is the same magnitude as the predefined bone graft dimension. In another embodiment, a distance that satisfies a predefined bone graft dimension can mean that the distance has a magnitude that is greater than the predefined bone graft dimension by some factor. In another embodiment, a distance that satisfies a predefined bone graft dimension can mean that the distance has a magnitude that is less than the predefined bone graft dimension by some factor. In another embodiment, a distance that satisfies a predefined bone graft dimension may not be exactly the same as the predefined dimension but falls within an acceptable margin of variation from the predefined dimension.

As discussed, in certain embodiments, the predefined bone graft dimension may be defined based on one or more of a model of bones of a patient, a model of an opening formed during a surgical procedure, an aspect of a patient-specific bone graft insertion site, and/or a model of a bone graft to be positioned between two bone fragments. However, in another embodiment the predefined bone graft dimension may be provided by a user. For example, the dimension needed may be a well-known and accepted magnitude for a particular surgical procedure. Alternatively, a surgeon may define the predefined bone graft dimension based on their professional judgment.

Embodiments according to the present disclosure enable a surgeon to preoperatively plan a surgical procedure, plan an autograft bone graft harvesting procedure, and plan and/or fabricate bone graft harvesting instruments, such as a patient-specific harvesting guide, with confidence that optimal bone material will be harvested, that the planned bone graft will be more readily resected, and that the planned bone graft positioned in a planned opening between one or more bone fragments will yield the desired correction and/or remediation the patient desires. A surgeon can proceed with scheduling and executing the surgical procedure knowing that harvesting a suitable autograft bone graft is not expected to be a trial-and-error procedure and will likely not include significant trimming or adjustment of the harvested autograft bone graft.

FIG. 13C is a bottom view of one embodiment of a harvesting guide 1246a. In certain embodiments, the harvesting guide 1246a may include one bone engagement surface 1270. Alternatively, or in addition, the harvesting guide 1246a may include one or more bone engagement surfaces 1270. In the illustrated embodiment, the bone engagement surface 1270 is on the back side 1254 (e.g., bone-facing side). The bone engagement surface 1270 is configured to engage a surface of a donor bone. In certain embodiments, the bone engagement surface 1270 is configured to engage one or more surfaces of a donor bone. In certain embodiments, the bone engagement surface 1270 is configured to engage at least two cortical surfaces of a donor bone. In certain embodiments, engagement by the bone engagement surface 1270 of a surface of a donor bone may mean that the bone engagement surface 1270 registers to or with the surface. In certain cases, a surgeon can feel the bone engagement surface 1270 “lock” or “slide” into place when a mirror contour of the bone engagement surface 1270 matches and engages with a corresponding contour of the donor bone surface.

In certain embodiments, the donor bone is a calcaneus 222. In particular, a surgeon may desire to harvest a bone graft from a lateral side of the calcaneus 222 behind the ankle and include cortical bone from the lateral cortex and the dorsal cortex. Given a typical topography of a calcaneus 222 in this area, matching a location selected in a preoperative plan with substantially the same location on a patient can be a challenge. However, because the bone engagement surface 1270 is configured to engage with more than one cortical surface, this challenge can be mitigated or overcome. Specifically, engaging more than one cortical surface can enhance how readily the bone engagement surface 1270 and/or harvesting guide 1246a engages with the surface of the donor bone.

In certain embodiments, the bone engagement surface 1270 can be used to position the harvesting guide 1246a in a desired position and/or against one or more bones and/or across one or more joints of a patient (a process referred to as registration, or registration to bone). The bone engagement surface 1270 contacting the patient anatomy can be contoured to mate with the patient anatomy. In one embodiment, the bone engagement surface 1270 includes a contour that is at least partially determined by a bone model of the donor bone. The bone model of the donor bone may be defined based on medical imaging of the donor bone. The donor bone may be bone of a patient of the surgical procedure.

In certain embodiments, the harvesting guide 1246a can include one or more landmark registration features 1282. In one embodiment, the one or more landmark registration features 1282 may extend from the back side 1254 and/or may extend between the back side 1254 and one of the top side 1256 and the bottom side 1258. The landmark registration features 1282 is configured to engage with a landmark of a donor bone. Those of skill in the art will appreciate that a bone engagement surface 1466 and/or landmark registration features 1282 can be positioned on any surface or side of the harvesting guide 1246a.

FIG. 13D is a left side view (i.e., posterior view) of the harvesting guide 1246a. FIG. 13D illustrates an example harvesting guide 1246a that is contoured to engage with both a lateral cortex surface of a donor bone (e.g., calcaneus 222) and a dorsal cortex surface of the donor bone.

FIG. 13E is a right-side view (i.e., anterior view) of the harvesting guide 1246a. Referring now to FIGS. 13D and 13E, the illustrated embodiment may include a third resection feature 1284 between a top side 1256 and a back side 1254 and a fourth resection feature 1286 between a bottom side 1258 and a back side 1254. The first resection feature 1264 extends from the front side 1252 to the back side 1254 and the second resection feature 1266 extends from the front side 1252 to the back side 1254.

In certain embodiments, the third resection feature 1284 and/or fourth resection feature 1286 may be implemented as an edge, or may include an edge, and rather than a slot or channel for guiding a cutting tool, the edge may serve as a guide for a surgeon performing an osteotomy. The edge (e.g., for a fourth resection feature 1286) may be an edge between the bottom side 1258 and the back side 1254. Another edge (e.g., for a third resection feature 1284) may be an edge between the top side 1256 and the back side 1254.

FIG. 13F is a top view (i.e., superior view) of the harvesting guide 1246a. FIG. 13G is a bottom view (i.e., inferior view) of the harvesting guide 1246a. Those of skill in the art will appreciate that a variety of designs may be designed and/or fabricated using embodiments of the present disclosure. Advantageously, the computing device and computer models and rapid prototype manufacturing and/or additive manufacturing systems and/or processes used in embodiments of the present disclosure enable significant flexibility and many design options for a harvesting guide 1246. A few alternative example embodiments are illustrated in FIGS. 13H-13N.

FIGS. 13H-13N are views of alternative embodiments of a harvesting guide according to certain embodiments. FIGS. 13H-13I illustrate harvesting guide 1246b according to one embodiment. FIG. 13J illustrates harvesting guide 1246c according to one embodiment. FIG. 13K illustrates harvesting guide 1246d according to one embodiment. FIGS. 13L-13N illustrate harvesting guide 1246e according to one embodiment. These alternative embodiments may have structures, features, functions, operations, and a configuration similar to or exactly the same as those of other harvesting guides described herein (e.g., harvesting guide 1246). Where these structures are substantially the same, like numerals are used to identify the corresponding parts. In the illustrated alternative embodiments, the harvesting guide may include one or more features that other embodiments may not include.

FIG. 13H illustrates a front side view harvesting guide 1246b that includes a first resection feature 1264 and a second resection feature 1266 offset from each other along front side 1252 by a predefined distance or dimension D1 that satisfies a predefined bone graft dimension. The offset D1 may be the same as an offset between first resection feature 1264 and second resection feature 1266 in the harvesting guide 1246a.

The harvesting guide 1246b may include one or more guard pin guides 1290. A guard pin guide 1290 serves to guide a guard pin that is deployed into a bone. In particular, a guard pin guide is configured to receive a guard pin that is deployed into bone, such as a donor bone. The guard pin prevents cutting of bone, such as a donor bone beyond a boundary. Generally, the guard pin guide 1290 guides the guard pin to enter the bone at or near the boundary. A guard pin in one embodiment of a guard. Often a guard pin is made of a material that resist cutting or breaking when contacted by a cutting tool. In one embodiment, the guard pin is a metal fastener such as a K-wire. Advantageously, the guard pin serves as a barrier to prevent cutting of bone on one side or past the guard pin, but permit cutting of bone on another side of the guard pin.

In the illustrated embodiment, the first resection feature 1264 has a closed end 1276 and second resection feature 1266 has a closed end 1280. The first resection feature 1264 includes a guard pin guide 1290 at, or near, the end 1274. Similarly, the second resection feature 1266 includes a guard pin guide 1290 at, or near, the end 1278. The diameter of the guard pin guide 1290 is sized to accommodate a guard pin that will be deployed through the guard pin guide 1290.

In certain embodiments, the guard pin guide 1290 extends through the body 1250 from a front side 1252 to a back side 1254. A guard pin guide 1290 may direct a deployed guard pin perpendicular to a bone surface or at a non-perpendicular angle to the bone surface. The angle or trajectory of the guard pin guide 1290 may be determined by the surgical procedure, the needs of the patient, and/or the discretion of the surgeon.

In certain embodiments, a harvesting guide 1246b may include a guard pin guide 1290 at an end of the first resection feature 1264 and/or second resection feature 1266. Alternatively, or in addition, the harvesting guide 1246b may include one or more guard pin guides 1290 at another position in the body 1250. For example, if the offset between the first resection feature 1264 and second resection feature 1266 is of a sufficient length, a user may opt to include another guard pin guide 1290 between the first resection feature 1264 and second resection feature 1266. This another guard pin guide 1290 may be aligned with a guard pin guide 1290a and a guard pin guide 1290b.

FIG. 13I illustrates a top side 1256 view of the alternative embodiment harvesting guide 1246b. In the illustrated embodiment, the harvesting guide 1246b includes a first resection feature 1264, second resection feature 1266, and third resection feature 1292. The third resection feature 1292 extends from a bone-facing side to a non-bone-facing side. In this embodiment, the third resection feature 1292a extends from the back side 1254 to the top side 1256.

In another embodiment, the third resection feature 1292b may extend between two sides different from the back side 1254 and top side 1256. For example, in FIG. 13J, the third resection feature 1292b extends from the front side 1252 to the back side 1254.

In general, where a harvesting guide 1246 includes a third resection feature 1292 serves to facilitate removal of a harvested bone graft. Depending on the bone graft harvest site position on the donor bone and/or the dimensions planned for the donor bone graft (e.g., autograft), a third resection feature 1292 may be helpful or may not be needed.

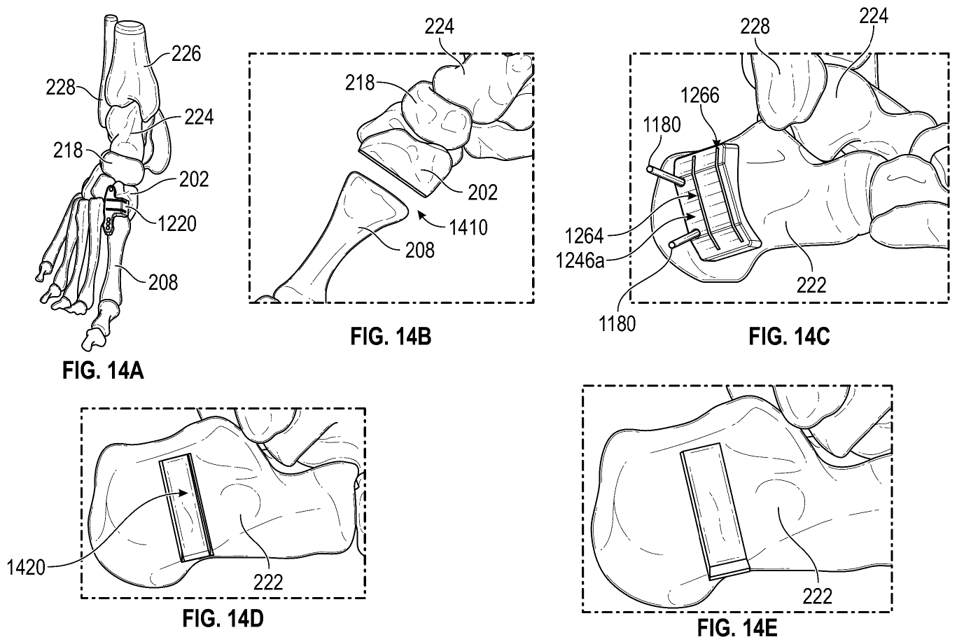

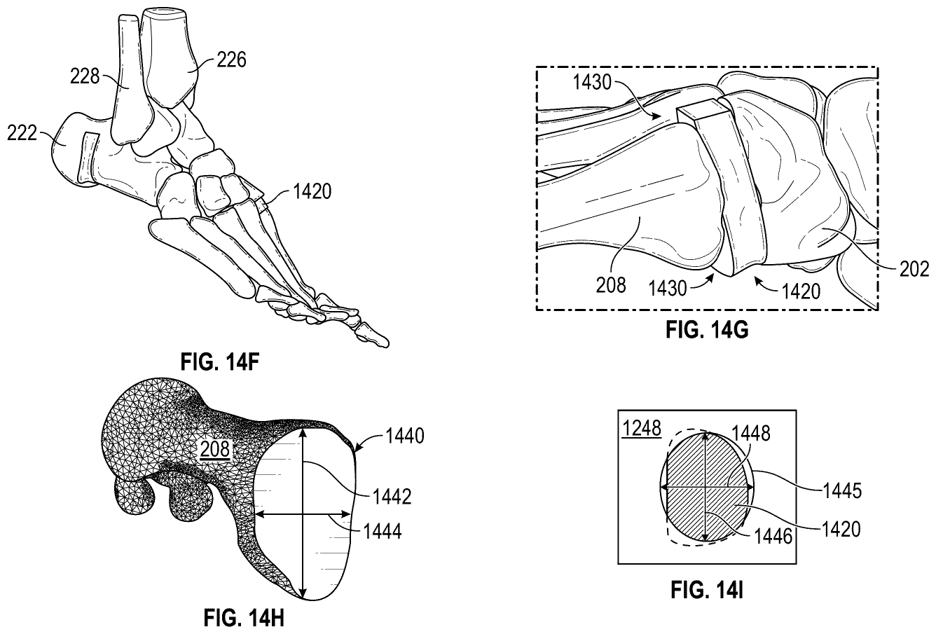

In one embodiment, where a harvesting guide 1246 includes a third resection feature 1292, the third resection feature 1292 may be configured to guide performing or creating a third osteotomy. In one embodiment, such as the harvesting guide 1246b illustrated, the third resection feature 1292a is configured to connect a first osteotomy formed using the first resection feature 1264 and a second osteotomy formed using the resection feature 1266. In other embodiments, the third resection feature 1292 may not connect osteotomies. In certain instances, a first osteotomy and a second osteotomy may provide planar cut faces and may leave only one more side of a bone graft connected to the donor bone. In such an instance, (e.g., FIGS. 14D, 14E), the third resection feature 1292a may enable a final, third osteotomy, which separates the bone graft from the donor bone. In another instance, a fourth osteotomy may be needed to separate the bone graft from the donor bone, such embodiments are also within the scope of the present disclosure.

In the illustrated embodiment, the third resection feature 1292a is implemented as a straight slot in the top side 1256 near the back side 1254. In this embodiment, the straight slot includes two closed ends. Of course, one of the ends may be open in another embodiment. Alternatively, or in addition, the harvesting guide 1246b can include an alternative version of a third resection feature 1292. Specifically, the third resection feature 1292b may be implemented by way of a an edge of the body 1250 between the top side 1256 and the back side 1254. In one embodiment, the edge may be straight. In another embodiment, the edge may be contoured. A surgeon may insert a cutting tool into donor bone at the edge and use the edge as a guide to complete an osteotomy.

FIG. 13J illustrates an alternative embodiment of a harvesting guide 1246. In this embodiment, the harvesting guide 1246c includes many of the same structures, features, and aspects of other embodiments. In addition, the harvesting guide 1246c includes an alternative version of a resection guide. In the illustrated embodiment, the harvesting guide 1246c includes a first resection feature 1264, a second resection feature 1266, and a third resection feature 1292c. The third resection feature 1292c extends from the front side 1252 to the back side 1254 and is positioned at or near end 1274 and end 1278 of the first resection feature 1264 and second resection feature 1266. The third resection feature 1292c is configured to connect a first osteotomy formed using the first resection feature 1264 and a second osteotomy formed using the second resection feature 1266.

FIG. 13K illustrates an embodiment of a harvesting guide 1246 similar to the harvesting guide 1246b illustrated in FIG. 13H. However, in this embodiment, the harvesting guide 1246d includes an alternative resection feature 1294. In certain embodiments, the alternative resection feature 1294 provides a similar function and/or benefit as a first resection feature 1264 and/or second resection feature 1266. The alternative resection feature 1294 may also include a guard pin guide 1290c like the first resection feature 1264 and second resection feature 1266.

In certain embodiments, such as harvesting guide 1246d, the alternative resection feature 1294 is offset from the first resection feature 1264 The predefined distance or dimension, offset D1, may be the same as an offset between first resection feature 1264 and second resection feature 1266 in the harvesting guide 1246a and/or harvesting guide 1246b. In addition, the alternative resection feature 1294 is offset from the first resection feature 1264 by a second dimension D2. In one embodiment, D2 is greater than D1. Alternatively, D2 can be smaller than D1.

In certain embodiments, a surgeon may request and/or the harvesting guide 1246d may include an alternative resection feature 1294 to provide more than one option for at least one dimension of a resected bone graft. For example, suppose a surgeon has concerns about the bone quality where at the bone graft harvest site. For example, the patient may have very porous or cancellous bone at the bone graft harvest site. Thus, the surgeon may want to extract a larger bone graft in an effort to get enough bone that will provide a suitable graft.

Alternatively, a surgeon may plan the osteotomies for a bone graft insertion site such that the span/depth/thickness of the opening may be one of two different possible dimensions. The actual size (e.g., span/depth/thickness) of the opening may depend on a decision a surgeon makes during an earlier stage of the surgical procedure. Thus, the surgeon may want two possible resection features in the harvesting guide 1246d that can be used depending on how the surgical procedure proceeds.

FIG. 13L illustrates a perspective view of a harvesting guide 1246e according to one embodiment. In this embodiment, the harvesting guide 1246e includes a single opening 1272 rather than two as used in harvesting guide 1246a for example. In addition, the first resection feature 1264 includes an open end 1276 and an open end 1280. FIG. 13M illustrates a front side view of the harvesting guide 1246e. FIG. 13N illustrates a back side view of the harvesting guide 1246e. FIG. 13N illustrates that this harvesting guide 1246e includes a bone engagement surface 1270 on the back side 1254.

FIG. 14A illustrates a perspective view of a foot and ankle of a patient including a medial cuneiform 202, first metatarsal 208, navicular 218, talus 224, tibia 226, and fibula 228. Advantageously, the embodiments of the present disclosure can be used to model each of the bones, instruments, openings, and/or grafts for a given surgical procedure before the procedure is initiated. The models can be used to design and/or fabricate instruments, physical models, and/or implants used in the surgical procedure. In one embodiment, as illustrated in FIGS. 14A-14G, the surgical procedure is a Lapidus surgical procedure that includes a harvesting of an autograft bone graft from a calcaneus 222 of the patient. Advantageously, a harvesting guide 1246 is provided since harvesting from a calcaneus 222 can be challenging due to the topography of the calcaneus 222.

FIG. 14A illustrates one example of a resection guide 1220 placed on a 1^{st} TMT joint of the patient. The resection guide 1220 can be coupled to the bones using bone attachment features that include fasteners 1180, e.g., K-wires. In one embodiment, a surgeon has formed a medial incision near the 1^{st} TMT joint with a medial approach. The surgeon has formed incisions in soft tissue down to the cortical bone surface of both a medial cuneiform 202 and a first metatarsal 208 of the TMT joint. Further, the surgeon has cut, or moved to the side, soft tissue covering the cortical bone surface of the bones sufficient to seat the resection guide 1220 onto the cortical bone surface of the bones and/or within the TMT joint. With the resection guide 1220 secured, the surgeon can make one or more cuts using resection guides of the resection guide 1220.

FIG. 14B illustrates a stage in the procedure after an osteotomy of the medial cuneiform 202 and the first metatarsal 208. The osteotomies have created an opening 1410 or gap 1410 between the distal end of the medial cuneiform 202 and the proximal end of the first metatarsal 208. Advantageously, using embodiments of the present disclosure, the size, shape, and/or dimensions of this opening 1410 can be measured or calculated within in model before the surgical procedure. From these measurements, predefined bone graft dimensions can be defined for a graft or implant that will be placed into the opening 1410. In certain surgical procedures it may be desirable to fill the opening 1410 with a graft and/or implant. Filling the opening 1410 can assist with providing desired biomechanics of an appendage, avoid undesirable aesthetics of a shortened set of bones, and/or provide other benefits.

In the illustrated embodiment, a surgeon plans to fill the opening 1410 with a graft, an autograft, harvested from a calcaneus 222 of the patient. FIG. 14C illustrates the calcaneus 222 with an example harvesting guide 1246 secured to the calcaneus 222 by bone attachment features that include fasteners 1180. Advantageously, the position on the calcaneus 222, size, dimensions for the harvesting guide 1246 as well as the size, shape, dimensions, and bone type composition of a graft that will be harvested using the harvesting guide 1246 have been preplanned, predesigned, and preconfigured such that a surgeon can rely on the harvesting guide 1246 to engage the calcaneus 222 in a predetermined position and guide one or more osteotomies of the calcaneus 222 to form the autograft 1420 (see FIG. 14D). In the illustrated embodiment, the harvesting guide 1246 is over a lateral cortex and wraps around to include a plantar cortex of the calcaneus 222. In this manner, the harvesting guide 1246 facilitates harvesting a bicortical autograft 1420.

FIG. 14D illustrates the calcaneus 222 after two osteotomies. The autograft 1420 is shown between two parallel cuts and is still connected on one side to the calcaneus 222. Next, a surgeon may use an osteotome to separate the autograft 1420 from the calcaneus 222. In certain embodiments, a surgeon may choose a bone graft harvest site in order to maximize the amount of cortical bone included in the autograft 1420. FIG. 14E illustrates the calcaneus 222 after a surgeon has removed the autograft 1420. Depending on the size of the autograft 1420 a surgeon may decide to fill the opening left in the calcaneus 222 by the removed autograft 1420.

In certain embodiments, a surgeon may choose to take allograft bone from a bone donor such as a cadaver. Advantageously, a surgeon may even use the harvesting guide 1246 to harvest the allograft from the cadaver. In such embodiments, the preoperative planning may include models and/or anatomic data for the cadaver. Alternatively, or in addition, conventional alternative methods can be used to fill in the opening left by the autograft 1420.

FIG. 14F illustrates the bones of the patient's foot with the autograft 1420 positioned within the opening 1410. Again, embodiments of the present disclosure can be used to position a model of the autograft 1420 in an opening between bones in the model. Using these models a technician, computing device, user, or surgeon can determine an optimal size, shape, dimensions, configuration, and orientation for the autograft 1420 in the opening 1410.

FIG. 14G is a close-up view of the autograft 1420 within the opening 1410 between the first metatarsal 208 and the medial cuneiform 202. Note that in this embodiment, the autograft 1420 is oriented such that the cortical surface of the autograft 1420 faces in a medial direction. In certain embodiments, the size and shape of the autograft 1420 may be sufficient for use in filling the opening 1410 with a graft that will serve the needs of patient, even though one or more parts 1430 (referred to as overhang) of the autograft 1420 may extend above or below the surfaces of bones adjacent to the opening 1410. These one or more parts 1430 may naturally fill in and join with and connect to the adjacent bones during postoperative healing. Next, a surgeon may fixate the first metatarsal 208, autograft 1420, and medial cuneiform 202 together using conventional fixation hardware and/or techniques including bone screws, bone plates, bone staples, or the like.

In certain embodiments, a surgeon and/or patient may desire an autograft 1420 that more closely matches the size and configuration of bones that connect to and fuse with the autograft 1420. FIG. 14H illustrates an end view of a first metatarsal 208 after an osteotomy near a proximal end of the first metatarsal 208. The osteotomy forms a cut face having an irregular shaped perimeter 1440 due to the shape of the first metatarsal 208. Advantageously, the embodiments of the present disclosure can be used to measure or calculate the shape of the irregular shaped perimeter 1440. This measured shape can be used to define a shape for a side of the autograft 1420 that will be joined to the first metatarsal 208. Alternatively, or in addition, an approximate shape may be measured or calculated by determining a length of a major axis 1442 and a minor axis 1444 for the irregular shaped perimeter 1440.

Referring to FIG. 14I, in certain embodiments, a jig 1148, such as jig 1248, can be designed using the embodiments of the present disclosure and fabricated to assist a surgeon or other user in shaping an autograft 1420 to a desired shape and/or configuration. In the illustrated embodiment, the jig 1248 can include an opening 1445 having a major axis 1446 and/or a minor axis 1448 that is substantially the same as, or slightly larger than the major axis 1442 and/or minor axis 1444. In the illustrated embodiment, the opening 1445 is an oval shape. In another embodiment, the computing device and/or models of bones involved in the surgical procedure can be used to define the opening 1445 to have substantially the same shape as a cross section of a cut face that will contact one side of the autograft 1420. In certain embodiments, the same jig 1248 is patient-specific.

In one embodiment, the jig 1248 can be used together with a burr or other cutting tool to shape an autograft 1420. The same jig 1248 is configured to guide shaping of a bone graft, such as an autograft 1420. Alternatively, or in addition, an opposite end of the autograft 1420 can be shaped using the same jig 1248 or a different jig 1248 configured based on a bone that will contact the opposite end of the autograft 1420 (e.g., a medial cuneiform 202).

In another embodiment, one or more jigs may be used. For example, in one embodiment, the harvesting guide 1246 can be configured to assist in making osteotomies for harvesting more than one autograft 1420. For example, the harvesting guide 1246 may include slots (resection features) for resecting to form two or more autografts 1420. In one embodiment, the two or more autografts 1420 may be deployed in an opening.

Alternatively, or in addition, the two or more autografts 1420 may be used together to form a single autograft that has more than two cortical surfaces, such as three, four, or has a circumferential cortical surface. For example, the present disclosure may provide one or more jigs that together can be used to form matching autografts that can be used to form a single autograft for use in the surgical procedure. For example, a first jig can be used to shape a first autograft into the shape of a Ying symbol. And a second jig can be used to shape a second autograft into the shape of a Yang symbol, where for the first autograft and the second autograft include a longer surface that is cortical bone. A user can then join the first autograft to the second autograft by bringing a noncortical surface of the Ying shaped first autograft near or to contact a noncortical surface of the Yang shaped second autograft to form a single autograft that has a circumferential surface made of cortical bone. Those of skill in the art will appreciate that rather than a Ying and Yang shape, the same ideas can be applied for joining two autografts in a puzzle piece, interlocking fashion to form a combined autograft to meet a particular need for a surgical procedure.

Advantageously, the embodiments of a harvesting guide as disclosed herein can greatly enhance a surgeon's ability to harvest a suitable autograft from a patient's donor bone such as a calcaneus. The harvesting guide can be used to make one or more of the cuts to resect the autograft. In certain embodiments, a harvesting guide may be used for more challenging cuts and other instruments, such as an osteotome, or a cutting tool without a guide may be used to make other cuts for the autograft.

Once the autograft is separated from the donor bone, one or more same jigs 1248 can be used to trim or make more precise adjustments to the shape and/or other dimensions of the autograft. In this manner, the present disclosure enables a surgeon to harvest an autograft that is sized to desired dimensions for use in filling an opening between two bones of the patient. Advantageously, the more precise and accurate an autograft the surgeon can harvest the more likely the patient will experience a successful outcome.

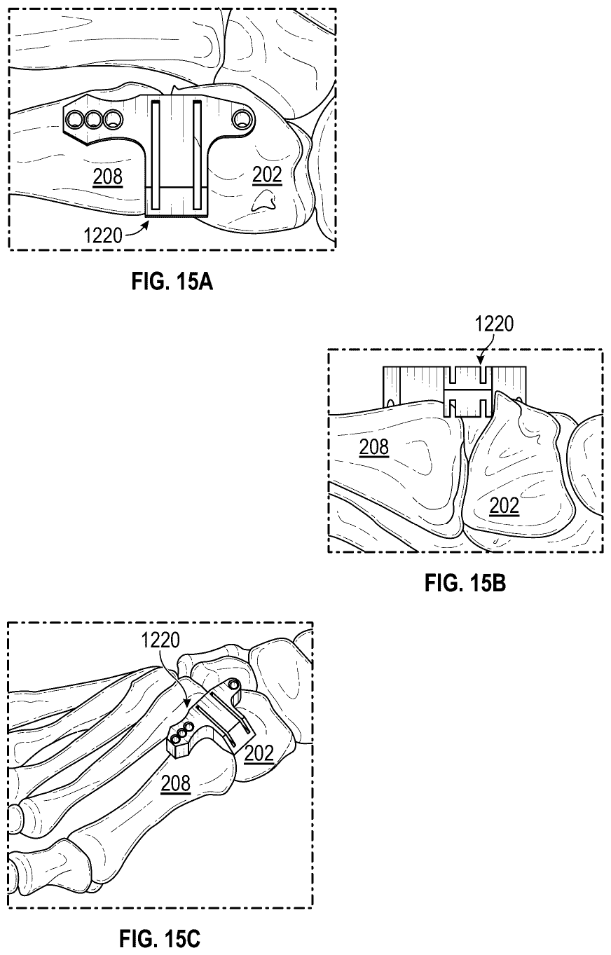

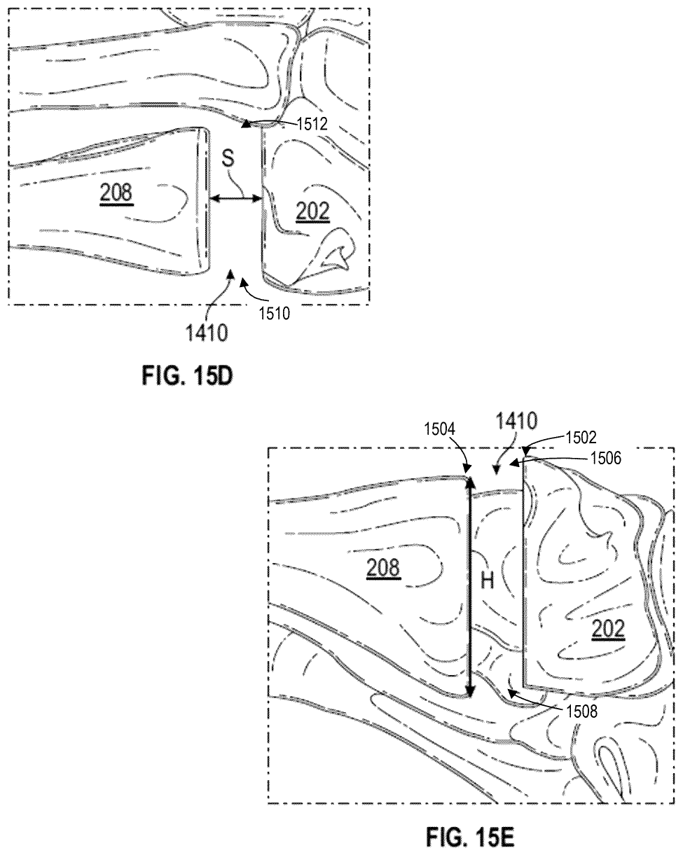

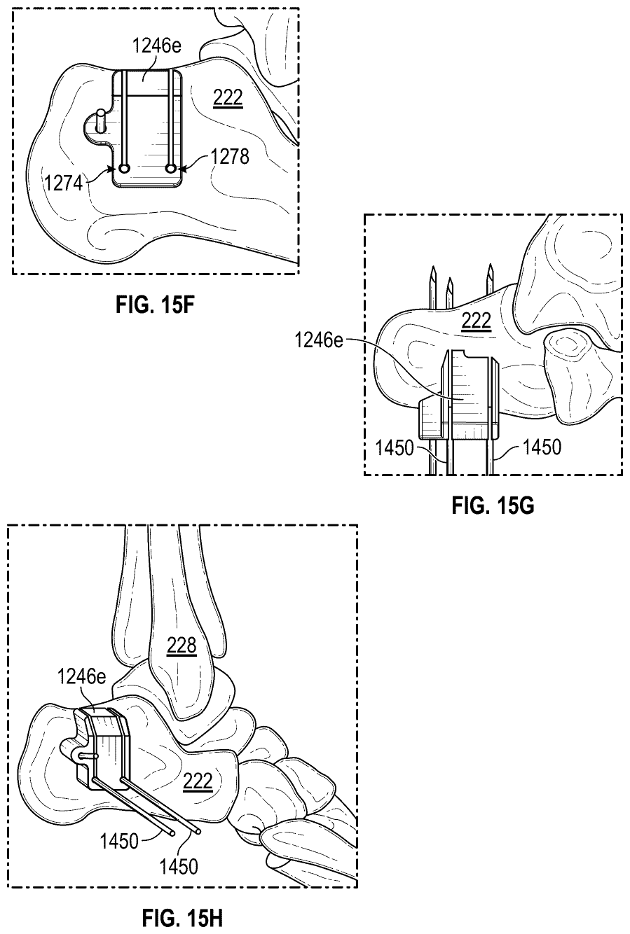

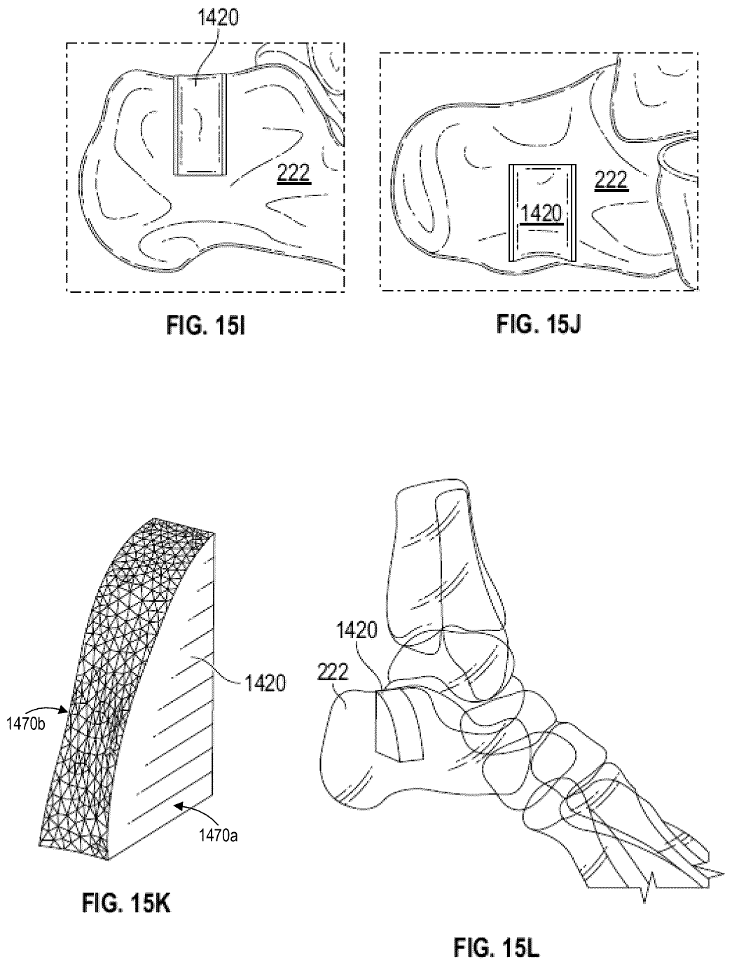

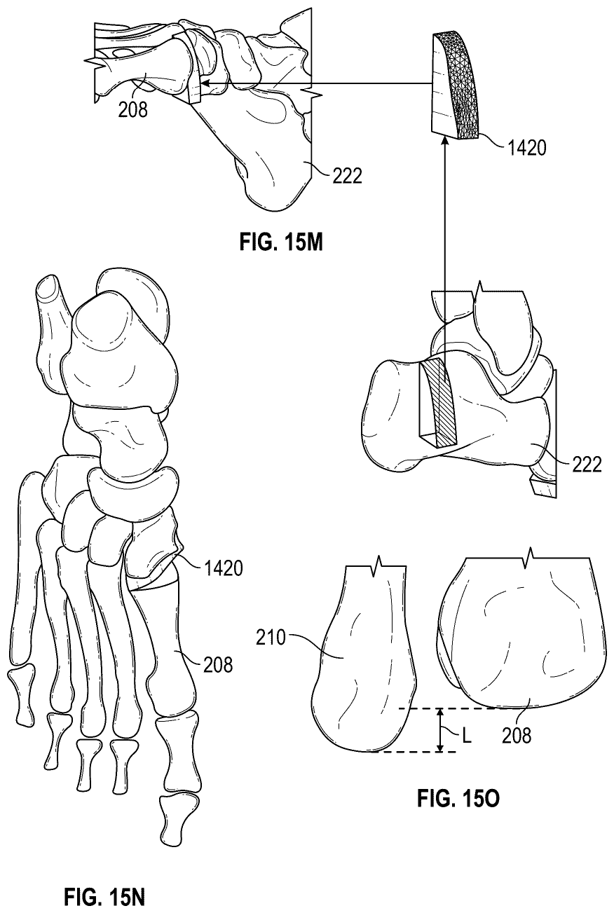

FIGS. 15A-15O illustrate different views and information pertinent to a planned surgical procedure that includes one or more osteotomies as well as harvesting of an autograft using a harvesting guide, such as a patient-specific harvesting guide. In certain embodiments, the views and information in FIGS. 15A-150 may be presented to a user using a computing device and a display device. In one embodiment, the computing device presents models of bones, instruments, and/or implants to a user so that preoperative planning and/or adjustments can be made. Thus, the bones illustrated in FIGS. 15A-150 may be models of bones of a patient.

Those of skill in the art will appreciate that these same images (e.g., FIGS. 15A-15O) may be used in a digital or hardcopy preoperative plan provided to a surgeon before the surgical procedure. These images can be made using models of patient bones as well as patient-specific instrumentation.

FIG. 15A illustrates a resection guide 1220 positioned on a TMT joint of a patient. FIG. 15B is a medial/lateral view of the resection guide 1220 on bones of the patient. FIG. 15C is a medial/lateral view of the resection guide 1220 on bones of the patient. Advantageously, the methods, systems, and/or apparatuses of the present disclosure can be used to design and/or fabricate the resection guide 1220 so that is it patient-specific and/or patient-matched.

FIG. 15D is a dorsal view of a model of the first metatarsal 208 and a model of the medial cuneiform 202, with an opening 1410 formed between them. FIG. 15D illustrates an opening 1410 that is to be formed using the resection guide 1220 during the surgical procedure. The opening 1410 may include a proximal end 1502, a distal end 1504, a dorsal end/side 1506, a plantar end/side 1508, a medial end/side 1510, and a lateral end/side 1512.

Note that because embodiments of the present disclosure are being used, a surgeon can see one or more dimensions for the opening 1410. In the illustrated embodiment, FIG. 15D indicates that the gap or opening 1410 has a span S that is approximately 10 mm in length, has a 10 mm depth.

FIG. 15E is a medial/medial-oblique view of the model first metatarsal 208 and the model medial cuneiform 202, with the opening 1410 between them. Similarly, FIG. 15E indicates that the gap or opening 1410 has a height (between the dorsal end 1506 and the plantar end 1508) of approximately 30 mm. This span/depth and height are examples of predefined bone graft dimensions that can be used to define dimensions for an autograft 1420. Accordingly, the harvesting guide 1246 can be fabricated to facilitate harvesting an autograft 1420 that has a length of 10 mm along one side and a height of 30 mm, at least on a distal end.

Referring to FIGS. 15D and 15E, in one example, a height on a proximal end 1502 of the opening 1410 can be is substantially the same as a height of the cut face of the medial cuneiform 202, a height on a distal end 1504 of the opening 1410 can be substantially the same as a height of the cut face of the first metatarsal 208.

Similarly, a length along a medial end 1510 of the opening 1410 can be substantially the same as a length between a medial side of the cut face of the medial cuneiform 202 and a medial side of the cut face of a first metatarsal 208, a length along a lateral end 1512 of the opening 1410 can be substantially the same as a length between a lateral side of the cut face of the medial cuneiform and a lateral side of the cut face of a first metatarsal. The lengths along a medial end 1520 and the lateral end 1512 may be the same or approximately the same as the span (e.g., length/depth/span/thickness) dimension for the opening 1410.

Similarly, a width of a proximal end 1502 of the opening 1410 can be substantially the same as a length between a medial side of the cut face of the medial cuneiform 202 and a lateral side of the cut face of the medial cuneiform 202 and a width of a distal end 1504 of the opening 1410 can be substantially the same as a length between a medial side of the cut face of the first metatarsal 208 and a lateral side of the cut face of the first metatarsal 208.

Advantageously, the dimensions of this opening 1410 (e.g., gap) are determined using a computing device and one or more models, anatomic data 412, and/or the like. In one embodiment, the opening 1410 can be defined based on bones of a patient positioned and oriented in their original position when the surgical procedure is initiated. The opening 1410 may be defined to be an opening formed by an osteotomy that resects a distal end of one bone of a joint and a proximal end of a second bone opposite the one bone and on an opposite side of the joint. A surgeon is provided the dimensions of this opening 1410 and can then determine what size bone graft is desired, should the surgeon want to fill the opening 1410 with a bone graft. Alternatively, or in addition, a surgeon can modify the size (e.g., dimensions) of the opening 1410 for any reason and use revised opening dimensions to plan for a bone graft. Specifically, at least one dimension for a bone graft can be predetermined and defined and a harvesting guide designed and/or fabricated to harvest a bone graft having that at least one dimension. Thus, a surgeon during preparation and planning for the surgical procedure has control of one or more dimensions for a bone graft to be harvested, and in particular, for a bone graft harvested from donor bone of the patient.

FIG. 15F illustrates a calcaneus 222 of a patient with a harvesting guide 1246 secured to a lateral side of the calcaneus 222. The harvesting guide 1246e includes resection features (aka slots) that can guide a surgeon in making one or more osteotomies to harvest the autograft 1420.

The embodiment of the harvesting guide 1246e in FIG. 15F includes holes near the first end 1274 and third end 1278. These holes are one example of guard pin guides 1290. The holes may accept a fastener that can serve as a guard or a guard pin 1450 (See FIG. 15H).

Note that this example harvesting guide 1246e is configured to harvest an autograft 1420 from both a dorsal cortex and a lateral cortex of the calcaneus 222. The resulting autograft 1420 will be a bicortical autograft 1420. It should also be noted that the harvesting guide 1246e in the illustrated embodiment includes resection guides that are open on the plantar side (second end 1276 and/or fourth end 1280). Those of skill in the art will appreciate that the resection features can be closed on one end or the other or open on both ends or open on one end or the other.

FIG. 15G illustrates a top-down (e.g., dorsal perspective) view of the harvesting guide 1246e and shows the harvesting guide 1246e secured to the calcaneus 222 and includes guard pins 1450 deployed.

FIG. 15H illustrates a perspective view of the harvesting guide 1246e secured to the calcaneus 222 and includes guard pins 1450 deployed. In one embodiment, a surgeon can remove the harvesting guide 1246e after making an osteotomy using two parallel resection features (e.g., first resection feature 1264, second resection feature 1266). The surgeon may leave the guard pins 1450 in place and use them to guide a horizontal cut that goes from one vertical cut to another vertical cut. The guard pins 1450 can help a surgeon keep a cutting tool from cutting more plantar than desired. Alternatively, or in addition, a surgeon may use holes for the guard pins 1450 to drill holes in the calcaneus 222 that can be used to guide forming a cut between the drilled holes in the calcaneus 222 using an osteotome or another cutting tool.

FIG. 15I illustrates the calcaneus 222 with the osteotomies completed and the harvesting guide 1246e removed. The example autograft 1420 is identified. FIG. 15J illustrates a top-down view of the calcaneus 222 with the example autograft 1420 still connected to the calcaneus 222.

FIG. 15K illustrates an example autograft 1420 separate and removed from the calcaneus 222 of FIG. 15L. The autograft 1420 includes one side that is made up of part of the dorsal cortex of the calcaneus 222 and part of the lateral cortex of the calcaneus 222. Advantageously, the autograft 1420 includes two opposing planar cut faces 1470a,b that can be used to fuse with planar cut faces formed by one or more osteotomies of bones of the patient. The ability of the harvesting guide 1246 according to certain embodiments of the present disclosure to provide a uniform planar cut face 1470a that is parallel to a uniform planar cut face 1470a can provide a surgeon with increased confidence that a planned surgical procedure will result in a desired positive outcome. The surgeon can implement adjustments and/or corrections to anatomy of the patient with other steps, instruments, and/or implants of the surgical procedure knowing that the autograft 1420 will have the desired dimensions and desired uniformity to serve its purpose in the surgical procedure. FIG. 15L illustrates the example autograft 1420 with other bones of the patient foot shown in transparent view.

FIG. 15M illustrates an example of using the autograft 1420 from the calcaneus 222 to fill the opening 1410 between the first metatarsal 208 and medial cuneiform 202 formed by an osteotomy. Advantageously, the dimensions for the autograft 1420 are predetermined and are accomplished by using a customized harvesting guide 1246e for harvesting the autograft 1420. One dimension for the autograft 1420 (and/or spacing of resection features used to harvest the autograft 1420) that directly impacts a distance between two bone fragments that will be fused to the autograft 1420 may be selected to satisfy a predefined bone graft dimension.

FIG. 15N illustrates the autograft 1420 deployed between the first metatarsal 208 and medial cuneiform 202. FIG. 15O illustrates an overall change in length L of a first metatarsal 208 relative to a second metatarsal 210 as a result of deploying the autograft 1420 for a surgical procedure. This change in length L may be reviewed by a surgeon prior to finalizing a preoperative plan. If the size of length L is acceptable, the preoperative plan may be finalized, and instrumentation fabricated. If the size of length L is unacceptable, a surgeon or technician may revise the preoperative plan until the size of length L is acceptable. Revising the preoperative plan may include changing a dimension, such as increasing a span, for the bone graft to decrease the size of length L.

Advantageously, embodiments of the present disclosure solves the challenge of how to harvest a graft, such as an autograft that has the desired dimensions. The embodiments of the present disclosure reduce time in the operating room, reduce the risk of cutting a graft too small, eliminate the need to resect an oversized graft that is then shaped and made smaller to fit a desired size for a surgical procedure. Advantageously, the dimensions of an opening at an osteotomy may be successfully transferred or mapped to a harvest site and used to resect a graft sized to meet the needs of the surgical procedure. The embodiments of the present disclosure minimize or eliminate the need for a surgeon to make free hand osteotomies when harvesting a graft. Furthermore, a harvesting guide according to one embodiment may be predefined and/or patient-specific to harvest a graft with predefined bone graft dimensions. In certain embodiments, the harvesting guide can include a stop that prevents a cutting tool from going past a certain depth in a donor bone. The stop can be defined by a distance between a front surface and back surface of the harvesting guide.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Alternatively, or in addition, any of the systems, devices, and/or apparatuses herein can be implemented using fewer than the elements and/or components described in the presented embodiments. In addition, components and/or elements and/or structures in one embodiment may be used in other embodiments to replace a component or structure and/or to augment the embodiment within the scope of the claims and present disclosure.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from it spirit and scope.

1. An apparatus for harvesting bone for a surgical procedure comprising:
a body;
a bone attachment feature;
a first resection feature configured to guide a first osteotomy of a donor bone;
a second resection feature configured to guide a second osteotomy of the donor bone, the second resection feature being offset from the first resection feature by a dimension set according to a harvesting guide model to provide a predefined bone graft dimension.

2. The apparatus of claim 1, wherein the predefined bone graft dimension is one of a set of predefined dimensions determined for an opening in a planned surgical procedure.

3. The apparatus of claim 1, wherein the body comprises a bone engagement surface configured to engage a surface of the donor bone.

4. The apparatus of claim 3, wherein the bone engagement surface comprises a contour at least partially determined based on a bone model of the donor bone, the bone model defined based on medical imaging of the donor bone.

5. The apparatus of claim 3, wherein the bone engagement surface is configured to engage at least two cortical surfaces of the donor bone.

6. The apparatus of claim 1, further comprising a landmark registration feature configured to engage a landmark of the donor bone.

7. The apparatus of claim 1, further comprising a guard pin guide configured to receive a guard pin into the donor bone such that the guard pin prevent cutting donor bone beyond a boundary.

8. The apparatus of claim 7, wherein the guard pin guide is positioned at an end of at least one of the first resection feature and the second resection feature.

9. The apparatus of claim 1, further comprising an alternative resection feature that is offset from the first resection feature by a second dimension, the second dimension different from the dimension.

10. The apparatus of claim 1, wherein the first resection feature and the second resection feature each extend through the body from a bone-facing side of the body to a non-bone-facing side of the body and wherein the first resection feature and the second resection feature extend through the body parallel to each other.

11. The apparatus of claim 10, wherein the first resection feature and the second resection feature extend through the body at an angle such that the first osteotomy and the second osteotomy form a wedge-shaped bone graft.

12. The apparatus of claim 1, wherein the donor bone is a calcaneus of a patient of a Lapidus surgical procedure.

13. A system for harvesting bone for a surgical procedure comprising:
a harvesting guide comprising:
a body;
a bone attachment feature;
a first resection feature, the first resection feature configured to guide a first osteotomy of a donor bone; and
a second resection feature, the second resection feature configured to guide a second osteotomy of the donor bone, the first resection feature extending through the body parallel to the second resection feature, the first resection feature separated from the second resection feature by a distance defined based on a patient-specific bone graft insertion site; and
a guard configured to prevent cutting the donor bone beyond a boundary.

14. The system of claim 13, wherein the harvesting guide comprises:
a third resection feature that extends from a bone-facing side to a non-bone-facing side of the body;
wherein the third resection feature guides a third osteotomy that connects the first osteotomy and the second osteotomy.

15. The system of claim 14, wherein the third resection feature comprises an edge.

16. The system of claim 13, further comprising a jig configured to guide shaping of a bone graft resected from the donor bone using the harvesting guide.

17. A method for harvesting a bone graft for a surgical procedure, the method comprising:
performing an osteotomy on a patient using an instrument that forms an opening having a set of predefined dimensions within one or more bones of a patient;
deploying a harvesting guide on to a donor bone, the harvesting guide comprising:
a body;
a bone attachment feature;
a first resection feature, the first resection feature configured to guide an osteotomy of the donor bone;
a second resection feature, the second resection feature configured to guide an osteotomy of the donor bone; and
wherein the first resection feature and second resection feature are configured relative to each other such that an osteotomy formed using the first resection feature and an osteotomy formed using the second resection feature facilitates resection of a bone graft having at least one dimension that substantially matches a dimension of the set of predefined dimensions; and
resecting the bone graft from the donor bone using the first resection feature and the second resection feature of the harvesting guide.

18. The method of claim 17, further comprising:
preparing the bone graft for reduction within the opening; and
reducing at least one bone fragment with the bone graft within the opening.

19. The method of claim 17, wherein the body comprises a bone engagement surface configured to engage at least two cortical surfaces of the donor bone.

20. A method for providing a patient-specific harvesting guide, the method comprising:
providing a set of predefined dimensions for an opening that is to receive a bone graft, the set of predefined dimensions unique to a patient;
determining a position on a donor bone for a bone graft harvest site;
developing a patient-specific harvesting guide model comprising a bone engagement surface configured to engage with a surface of the bone graft harvest site;
fabricating a patient-specific harvesting guide based on the patient-specific harvesting guide model; and
providing the patient-specific harvesting guide for a surgical procedure.
